SEQUENCE LISTING (28 sequences)

SEQ ID NO: 1 - DNA, 772 bp, Artificial
  Synthetic OriV of pBBR1 plasmid
gcttatctccatgcggtaggggtgccgcacggttgcggcaccatgcgcaatcagctgcaacttttcggcagcgcgacaacaattatgcgttgcgtaaaagtggcagtcaattacagattttctttaacctacgcaatgagctattgcggggggtgccgcaatgagctgttgcgtaccccccttttttaagttgttgatttttaagtctttcgcatttcgccctatatctagttctttggtgcccaaagaagggcacccctgcggggttcccccacgccttcggcgcggctccccctccggcaaaaagtggcccctccggggcttgttgatcgactgcgcggccttcggccttgcccaaggtggcgctgcccccttggaacccccgcactcgccgccgtgaggctcggggggcaggcgggcgggcttcgcccttcgactgcccccactcgcataggcttgggtcgttccaggcgcgtcaaggccaagccgctgcgcggtcgctgcgcgagccttgacccgccttccacttggtgtccaaccggcaagcgaagcgcgcaggccgcaggccggaggcttttccccagagaaaattaaaaaaattgatggggcaaggccgcaggccgcgcagttggagccggtgggtatgtggtcgaaggctgggtagccggtgggcaatccctgtggtcaagctcgtgggcaggcgcagcctgtccatcagcttgtccagcagggttgtccacgggccgagcgaagcgagccagccggtggccgc

SEQ ID NO: 2 - DNA, 109 bp, Artificial
  Synthetic OriT of RP4 plasmid
gggcaggataggtgaagtaggcccacccgcgagcgggtgttccttcttcactgtcccttattcgcacctggcggtgctcaacgggaatcctgctctgcgaggctggccg

SEQ ID NO: 3 - DNA, 290 bp, Artificial
  Synthetic oriT of F plasmid
aggctcaacaggttggtggttctcaccaccaaaagcaccacaccccacgcaaaaacaagtttttgctgatttttctttataaatagagtgttatgaaaaattagtttctcttactctctttatgatatttaaaaaagcggtgtcggcgcggctacaacaacgcgccgacaccgttttgtaggggtggtactgactatttttataaaaaacattattttatattaggggtgctgctagcggcgcggtgtgtttttttataggataccgctaggggcgctgctagcggtgcg

SEQ ID NO: 4 - protein, 1368 aa, Streptococcus pyogenes
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 5 - protein, 1368 aa, Artificial; identical to SEQ ID NO: 4
  Synthetic Nickase

SEQ ID NO: 6 - protein, 1368 aa, Artificial
  Synthetic Nickase
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 7 - DNA, 4107 bp, Artificial
  Synthetic Cas9 (addgene #44250)
atggataagaaatactcaataggcttagatatcggcacaaatagcgtcggatgggcggtgatcactgatgaatataaggttccgtctaaaaagttcaaggttctgggaaatacagaccgccacagtatcaaaaaaaatcttataggggctcttttatttgacagtggagagacagcggaagcgactcgtctcaaacggacagctcgtagaaggtatacacgtcggaagaatcgtatttgttatctacaggagattttttcaaatgagatggcgaaagtagatgatagtttctttcatcgacttgaagagtcttttttggtggaagaagacaagaagcatgaacgtcatcctatttttggaaatatagtagatgaagttgcttatcatgagaaatatccaactatctatcatctgcgaaaaaaattggtagattctactgataaagcggatttgcgcttaatctatttggccttagcgcatatgattaagtttcgtggtcattttttgattgagggagatttaaatcctgataatagtgatgtggacaaactatttatccagttggtacaaacctacaatcaattatttgaagaaaaccctattaacgcaagtggagtagatgctaaagcgattctttctgcacgattgagtaaatcaagacgattagaaaatctcattgctcagctccccggtgagaagaaaaatggcttatttgggaatctcattgctttgtcattgggtttgacccctaattttaaatcaaattttgatttggcagaagatgctaaattacagctttcaaaagatacttacgatgatgatttagataatttattggcgcaaattggagatcaatatgctgatttgtttttggcagctaagaatttatcagatgctattttactttcagatatcctaagagtaaatactgaaataactaaggctcccctatcagcttcaatgattaaacgctacgatgaacatcatcaagacttgactcttttaaaagctttagttcgacaacaacttccagaaaagtataaagaaatcttttttgatcaatcaaaaaacggatatgcaggttatattgatgggggagctagccaagaagaattttataaatttatcaaaccaattttagaaaaaatggatggtactgaggaattattggtgaaactaaatcgtgaagatttgctgcgcaagcaacggacctttgacaacggctctattccccatcaaattcacttgggtgagctgcatgctattttgagaagacaagaagacttttatccatttttaaaagacaatcgtgagaagattgaaaaaatcttgacttttcgaattccttattatgttggtccattggcgcgtggcaatagtcgttttgcatggatgactcggaagtctgaagaaacaattaccccatggaattttgaagaagttgtcgataaaggtgcttcagctcaatcatttattgaacgcatgacaaactttgataaaaatcttccaaatgaaaaagtactaccaaaacatagtttgctttatgagtattttacggtttataacgaattgacaaaggtcaaatatgttactgaaggaatgcgaaaaccagcatttctttcaggtgaacagaagaaagccattgttgatttactcttcaaaacaaatcgaaaagtaaccgttaagcaattaaaagaagattatttcaaaaaaatagaatgttttgatagtgttgaaatttcaggagttgaagatagatttaatgcttcattaggtacctaccatgatttgctaaaaattattaaagataaagattttttggataatgaagaaaatgaagatatcttagaggatattgttttaacattgaccttatttgaagatagggagatgattgaggaaagacttaaaacatatgctcacctctttgatgataaggtgatgaaacagcttaaacgtcgccgttatactggttggggacgtttgtctcgaaaattgattaatggtattagggataagcaatctggcaaaacaatattagattttttgaaatcagatggttttgccaatcgcaattttatgcagctgatccatgatgatagtttgacatttaaagaagacattcaaaaagcacaagtgtctggacaaggcgatagtttacatgaacatattgcaaatttagctggtagccctgctattaaaaaaggtattttacagactgtaaaagttgttgatgaattggtcaaagtaatggggcggcataagccagaaaatatcgttattgaaatggcacgtgaaaatcagacaactcaaaagggccagaaaaattcgcgagagcgtatgaaacgaatcgaagaaggtatcaaagaattaggaagtcagattcttaaagagcatcctgttgaaaatactcaattgcaaaatgaaaagctctatctctattatctccaaaatggaagagacatgtatgtggaccaagaattagatattaatcgtttaagtgattatgatgtcgatcacattgttccacaaagtttccttaaagacgattcaatagacaataaggtcttaacgcgttctgataaaaatcgtggtaaatcggataacgttccaagtgaagaagtagtcaaaaagatgaaaaactattggagacaacttctaaacgccaagttaatcactcaacgtaagtttgataatttaacgaaagctgaacgtggaggtttgagtgaacttgataaagctggttttatcaaacgccaattggttgaaactcgccaaatcactaagcatgtggcacaaattttggatagtcgcatgaatactaaatacgatgaaaatgataaacttattcgagaggttaaagtgattaccttaaaatctaaattagtttctgacttccgaaaagatttccaattctataaagtacgtgagattaacaattaccatcatgcccatgatgcgtatctaaatgccgtcgttggaactgctttgattaagaaatatccaaaacttgaatcggagtttgtctatggtgattataaagtttatgatgttcgtaaaatgattgctaagtctgagcaagaaataggcaaagcaaccgcaaaatatttcttttactctaatatcatgaacttcttcaaaacagaaattacacttgcaaatggagagattcgcaaacgccctctaatcgaaactaatggggaaactggagaaattgtctgggataaagggcgagattttgccacagtgcgcaaagtattgtccatgccccaagtcaatattgtcaagaaaacagaagtacagacaggcggattctccaaggagtcaattttaccaaaaagaaattcggacaagcttattgctcgtaaaaaagactgggatccaaaaaaatatggtggttttgatagtccaacggtagcttattcagtcctagtggttgctaaggtggaaaaagggaaatcgaagaagttaaaatccgttaaagagttactagggatcacaattatggaaagaagttcctttgaaaaaaatccgattgactttttagaagctaaaggatataaggaagttaaaaaagacttaatcattaaactacctaaatatagtctttttgagttagaaaacggtcgtaaacggatgctggctagtgccggagaattacaaaaaggaaatgagctggctctgccaagcaaatatgtgaattttttatatttagctagtcattatgaaaagttgaagggtagtccagaagataacgaacaaaaacaattgtttgtggagcagcataagcattatttagatgagattattgagcaaatcagtgaattttctaagcgtgttattttagcagatgccaatttagataaagttcttagtgcatataacaaacatagagacaaaccaatacgtgaacaagcagaaaatattattcatttatttacgttgacgaatcttggagctcccgctgcttttaaatattttgatacaacaattgatcgtaaacgatatacgtctacaaaagaagttttagatgccactcttatccatcaatccatcactggtctttatgaaacacgcattgatttgagtcagctaggaggtgactaa

SEQ ID NO: 8 - DNA, 4107 bp, Artificial
  Synthetic dCas9 (addgene #44249)
atggataagaaatactcaataggcttagctatcggcacaaatagcgtcggatgggcggtgatcactgatgaatataaggttccgtctaaaaagttcaaggttctgggaaatacagaccgccacagtatcaaaaaaaatcttataggggctcttttatttgacagtggagagacagcggaagcgactcgtctcaaacggacagctcgtagaaggtatacacgtcggaagaatcgtatttgttatctacaggagattttttcaaatgagatggcgaaagtagatgatagtttctttcatcgacttgaagagtcttttttggtggaagaagacaagaagcatgaacgtcatcctatttttggaaatatagtagatgaagttgcttatcatgagaaatatccaactatctatcatctgcgaaaaaaattggtagattctactgataaagcggatttgcgcttaatctatttggccttagcgcatatgattaagtttcgtggtcattttttgattgagggagatttaaatcctgataatagtgatgtggacaaactatttatccagttggtacaaacctacaatcaattatttgaagaaaaccctattaacgcaagtggagtagatgctaaagcgattctttctgcacgattgagtaaatcaagacgattagaaaatctcattgctcagctccccggtgagaagaaaaatggcttatttgggaatctcattgctttgtcattgggtttgacccctaattttaaatcaaattttgatttggcagaagatgctaaattacagctttcaaaagatacttacgatgatgatttagataatttattggcgcaaattggagatcaatatgctgatttgtttttggcagctaagaatttatcagatgctattttactttcagatatcctaagagtaaatactgaaataactaaggctcccctatcagcttcaatgattaaacgctacgatgaacatcatcaagacttgactcttttaaaagctttagttcgacaacaacttccagaaaagtataaagaaatcttttttgatcaatcaaaaaacggatatgcaggttatattgatgggggagctagccaagaagaattttataaatttatcaaaccaattttagaaaaaatggatggtactgaggaattattggtgaaactaaatcgtgaagatttgctgcgcaagcaacggacctttgacaacggctctattccccatcaaattcacttgggtgagctgcatgctattttgagaagacaagaagacttttatccatttttaaaagacaatcgtgagaagattgaaaaaatcttgacttttcgaattccttattatgttggtccattggcgcgtggcaatagtcgttttgcatggatgactcggaagtctgaagaaacaattaccccatggaattttgaagaagttgtcgataaaggtgcttcagctcaatcatttattgaacgcatgacaaactttgataaaaatcttccaaatgaaaaagtactaccaaaacatagtttgctttatgagtattttacggtttataacgaattgacaaaggtcaaatatgttactgaaggaatgcgaaaaccagcatttctttcaggtgaacagaagaaagccattgttgatttactcttcaaaacaaatcgaaaagtaaccgttaagcaattaaaagaagattatttcaaaaaaatagaatgttttgatagtgttgaaatttcaggagttgaagatagatttaatgcttcattaggtacctaccatgatttgctaaaaattattaaagataaagattttttggataatgaagaaaatgaagatatcttagaggatattgttttaacattgaccttatttgaagatagggagatgattgaggaaagacttaaaacatatgctcacctctttgatgataaggtgatgaaacagcttaaacgtcgccgttatactggttggggacgtttgtctcgaaaattgattaatggtattagggataagcaatctggcaaaacaatattagattttttgaaatcagatggttttgccaatcgcaattttatgcagctgatccatgatgatagtttgacatttaaagaagacattcaaaaagcacaagtgtctggacaaggcgatagtttacatgaacatattgcaaatttagctggtagccctgctattaaaaaaggtattttacagactgtaaaagttgttgatgaattggtcaaagtaatggggcggcataagccagaaaatatcgttattgaaatggcacgtgaaaatcagacaactcaaaagggccagaaaaattcgcgagagcgtatgaaacgaatcgaagaaggtatcaaagaattaggaagtcagattcttaaagagcatcctgttgaaaatactcaattgcaaaatgaaaagctctatctctattatctccaaaatggaagagacatgtatgtggaccaagaattagatattaatcgtttaagtgattatgatgtcgatgccattgttccacaaagtttccttaaagacgattcaatagacaataaggtcttaacgcgttctgataaaaatcgtggtaaatcggataacgttccaagtgaagaagtagtcaaaaagatgaaaaactattggagacaacttctaaacgccaagttaatcactcaacgtaagtttgataatttaacgaaagctgaacgtggaggtttgagtgaacttgataaagctggttttatcaaacgccaattggttgaaactcgccaaatcactaagcatgtggcacaaattttggatagtcgcatgaatactaaatacgatgaaaatgataaacttattcgagaggttaaagtgattaccttaaaatctaaattagtttctgacttccgaaaagatttccaattctataaagtacgtgagattaacaattaccatcatgcccatgatgcgtatctaaatgccgtcgttggaactgctttgattaagaaatatccaaaacttgaatcggagtttgtctatggtgattataaagtttatgatgttcgtaaaatgattgctaagtctgagcaagaaataggcaaagcaaccgcaaaatatttcttttactctaatatcatgaacttcttcaaaacagaaattacacttgcaaatggagagattcgcaaacgccctctaatcgaaactaatggggaaactggagaaattgtctgggataaagggcgagattttgccacagtgcgcaaagtattgtccatgccccaagtcaatattgtcaagaaaacagaagtacagacaggcggattctccaaggagtcaattttaccaaaaagaaattcggacaagcttattgctcgtaaaaaagactgggatccaaaaaaatatggtggttttgatagtccaacggtagcttattcagtcctagtggttgctaaggtggaaaaagggaaatcgaagaagttaaaatccgttaaagagttactagggatcacaattatggaaagaagttcctttgaaaaaaatccgattgactttttagaagctaaaggatataaggaagttaaaaaagacttaatcattaaactacctaaatatagtctttttgagttagaaaacggtcgtaaacggatgctggctagtgccggagaattacaaaaaggaaatgagctggctctgccaagcaaatatgtgaattttttatatttagctagtcattatgaaaagttgaagggtagtccagaagataacgaacaaaaacaattgtttgtggagcagcataagcattatttagatgagattattgagcaaatcagtgaattttctaagcgtgttattttagcagatgccaatttagataaagttcttagtgcatataacaaacatagagacaaaccaatacgtgaacaagcagaaaatattattcatttatttacgttgacgaatcttggagctcccgctgcttttaaatattttgatacaacaattgatcgtaaacgatatacgtctacaaaagaagttttagatgccactcttatccatcaatccatcactggtctttatgaaacacgcattgatttgagtcagctaggaggtgactaa

SEQ ID NO: 9 - DNA, 53 bp, Artificial
  Synthetic promoter
tttacggctagctcagccctaggtattatgctagcagatctaaagaggagaaa

SEQ ID NO: 10 - DNA, 35 bp, Artificial
  Synthetic spacer sequence
tagtccattacggtcaatccgccgtttgttcccag

SEQ ID NO: 11 - DNA, 35 bp, Artificial
  Synthetic spacer sequence
aaactgggaacaaacggcggattgaccgtaatgga

SEQ ID NO: 12 - DNA, 25 bp, Artificial
  Synthetic spacer sequence
tagtcaccaaaaacacagccgatag

SEQ ID NO: 13 - DNA, 25 bp, Artificial
  Synthetic spacer sequence
aaactatcggctgtgtttttggtga

SEQ ID NO: 14 - DNA, 25 bp, Artificial
  Synthetic spacer sequence
tagttgtttttcatgttgtcacccg

SEQ ID NO: 15 - DNA, 25 bp, Artificial
  Synthetic spacer sequence
aaacgggtgacaacatgaaaaacaa

SEQ ID NO: 16 - DNA, 25 bp, Artificial
  Synthetic spacer sequence
tagttgtaatcacaatgatgaaaag

SEQ ID NO: 17 - DNA, 25 bp, Artificial
  Synthetic spacer sequence
aaacttttcatcattgtgattacaa

SEQ ID NO: 18 - DNA, 25 bp, Artificial
  Synthetic spacer sequence
tagtagcgccaaccgatatcaccag

SEQ ID NO: 19 - DNA, 25 bp, Artificial
  Synthetic spacer sequence
aaactggtgatatcggttggcgcta

SEQ ID NO: 20 - DNA, 22 bp, Artificial
  Synthetic spacer sequence
tagttgccatcactactgtgcg

SEQ ID NO: 21 - DNA, 22 bp, Artificial
  Synthetic spacer sequence
aaacgcacagtagtgatggcaa

SEQ ID NO: 22 - DNA, 22 bp, Artificial
  Synthetic space sequence
tagttggcgttgaagacgacgg

SEQ ID NO: 23 - DNA, 22 bp, Artificial
  Synthetic spacer sequence
aaaccgtcgtcttcaacgccaa

SEQ ID NO: 24 - DNA, 22 bp, Artificial
  Synthetic spacer sequence
tagtgatatcaaagtggttatg

SEQ ID NO: 25 - DNA, 76 bp, Artificial
  Synthetic tracrRNA sequence
gttttagagctagaaatagcaagttaaaataaggctagtccgttatcaacttgaaaaagtggcaccgagtcggtgc

SEQ ID NO: 26 - DNA, 35 bp, Artificial
  Synthetic promoter
ttgacagctagctcagtcctaggtataatactagt

SEQ ID NO: 27 - DNA, 7631 bp, Artificial
  Synthetic TAPKn-Cas9-nsp
atctttgacagctagctcagtcctaggtataatactagtgaagagcaccggtgctcttcgttttagagctagaaatagcaagttaaaataaggctagtccgttatcaacttgaaaaagtggcaccgagtcggtgctttttttgaagcttgggcccgaacaaaaaaaaaccccgcccctgacagggcggggttttttttgtttgtcggtgaactggatccttaccagctgggcgcgccccccctacgggcttgctctccgggcttcgccctgcgcggtcgctgcgctcccttgccagcccgtggatatgtggacgatggccgcgagcggccaccggctggctcgcttcgctcggcccgtggacaaccctgctggacaagctgatggacaggctgcgcctgcccacgagcttgaccacagggattgcccaccggctacccagccttcgaccacatacccaccggctccaactgcgcggcctgcggccttgccccatcaatttttttaattttctctggggaaaagcctccggcctgcggcctgcgcgcttcgcttgccggttggacaccaagtggaaggcgggtcaaggctcgcgcagcgaccgcgcagcggcttggccttgacgcgcctggaacgacccaagcctatgcgagtgggggcagtcgaagggcgaagcccgcccgcctgccccccgagcctcacggcggcgagtgcgggggttccaagggggcagcgccaccttgggcaaggccgaaggccgcgcagtcgatcaacaagccccggaggggccactttttgccggagggggagccgcgccgaaggcgtgggggaaccccgcaggggtgcccttctttgggcaccaaagaactagatatagggcgaaatgcgaaagacttaaaaatcaacaacttaaaaaaggggggtacgcaacagctcattgcggcaccccccgcaatagctcattgcgtaggttaaagaaaatctgtaattgactgccacttttacgcaacgcataattgttgtcgcgctgccgaaaagttgcagctgattgcgcatggtgccgcaaccgtgcggcacccctaccgcatggagataagcatggccacgcagtccagagaaatcggcattcaagccaagaacaagcccggtcactgggtgcaaacggaacgcaaagcgcatgaggcgtgggccgggcttattgcgaggaaacccacggcggcaatgctgctgcatcacctcgtggcgcagatgggccaccagaacgccgtggtggtcagccagaagacactttccaagctcatcggacgttctttgcggacggtccaatacgcagtcaaggacttggtggccgagcgctggatctccgtcgtgaagctcaacggccccggcaccgtgtcggcctacgtggtcaatgaccgcgtggcgtggggccagccccgcgaccagttgcgcctgtcggtgttcagtgccgccgtggtggttgatcacgacgaccaggacgaatcgctgttggggcatggcgacctgcgccgcatcccgaccctgtatccgggcgagcagcaactaccgaccggccccggcgaggagccgcccagccagcccggcattccgggcatggaaccagacctgccagccttgaccgaaacggaggaatgggaacggcgcgggcagcagcgcctgccgatgcccgatgagccgtgttttctggacgatggcgagccgttggagccgccgacacgggtaacgctgccgcgccggtagggcatgcaaacagggacgcaccgctagcagcgcccctagcggtatcctataaaaaaacacaccgcgccgctagcagcacccctaatataaaataatgttttttataaaaatagtcagtaccacccctacaaaacggtgtcggcgcgttgttgtagccgcgccgacaccgcttttttaaatatcataaagagagtaagagaaactaatttttcataacactctatttataaagaaaaatcagcaaaaacttgtttttgcgtggggtgtggtgcttttggtggtgagaaccaccaacctgttgagcctttttgtggagcgcgcggaccgcggtccagagagcgttcaccgacaaacaacagataaaacgaaaggcccagtctttcgactgagcctttcgttttatttgatgcctggagatccttactcgagttagtcacctcctagctgactcaaatcaatgcgtgtttcataaagaccagtgatggattgatggataagagtggcatctaaaacttcttttgtagacgtatatcgtttacgatcaattgttgtatcaaaatatttaaaagcagcgggagctccaagattcgtcaacgtaaataaatgaataatattttctgcttgttcacgtattggtttgtctctatgtttgttatatgcactaagaactttatctaaattggcatctgctaaaataacacgcttagaaaattcactgatttgctcaataatctcatctaaataatgcttatgctgctccacaaacaattgtttttgttcgttatcttctggactacccttcaacttttcataatgactagctaaatataaaaaattcacatatttgcttggcagagccagctcatttcctttttgtaattctccggcactagccagcatccgtttacgaccgttttctaactcaaaaagactatatttaggtagtttaatgattaagtcttttttaacttccttatatcctttagcttctaaaaagtcaatcggatttttttcaaaggaacttctttccataattgtgatccctagtaactctttaacggattttaacttcttcgatttccctttttccaccttagcaaccactaggactgaataagctaccgttggactatcaaaaccaccatatttttttggatcccagtcttttttacgagcaataagcttgtccgaatttctttttggtaaaattgactccttggagaatccgcctgtctgtacttctgttttcttgacaatattgacttggggcatggacaatactttgcgcactgtggcaaaatctcgccctttatcccagacaatttctccagtttccccattagtttcgattagagggcgtttgcgaatctctccatttgcaagtgtaatttctgttttgaagaagttcatgatattagagtaaaagaaatattttgcggttgctttgcctatttcttgctcagacttagcaatcattttacgaacatcataaactttataatcaccatagacaaactccgattcaagttttggatatttcttaatcaaagcagttccaacgacggcatttagatacgcatcatgggcatgatggtaattgttaatctcacgtactttatagaattggaaatcttttcggaagtcagaaactaatttagattttaaggtaatcactttaacctctcgaataagtttatcattttcatcgtatttagtattcatgcgactatccaaaatttgtgccacatgcttagtgatttggcgagtttcaaccaattggcgtttgataaaaccagctttatcaagttcactcaaacctccacgttcagctttcgttaaattatcaaacttacgttgagtgattaacttggcgtttagaagttgtctccaatagtttttcatctttttgactacttcttcacttggaacgttatccgatttaccacgatttttatcagaacgcgttaagaccttattgtctattgaatcgtctttaaggaaactttgtggaacaatgtgatcgacatcataatcacttaaacgattaatatctaattcttggtccacatacatgtctcttccattttggagataatagagatagagcttttcattttgcaattgagtattttcaacaggatgctctttaagaatctgacttcctaattctttgataccttcttcgattcgtttcatacgctctcgcgaatttttctggcccttttgagttgtctgattttcacgtgccatttcaataacgatattttctggcttatgccgccccattactttgaccaattcatcaacaacttttacagtctgtaaaataccttttttaatagcagggctaccagctaaatttgcaatatgttcatgtaaactatcgccttgtccagacacttgtgctttttgaatgtcttctttaaatgtcaaactatcatcatggatcagctgcataaaattgcgattggcaaaaccatctgatttcaaaaaatctaatattgttttgccagattgcttatccctaataccattaatcaattttcgagacaaacgtccccaaccagtataacggcgacgtttaagctgtttcatcaccttatcatcaaagaggtgagcatatgttttaagtctttcctcaatcatctccctatcttcaaataaggtcaatgttaaaacaatatcctctaagatatcttcattttcttcattatccaaaaaatctttatctttaataatttttagcaaatcatggtaggtacctaatgaagcattaaatctatcttcaactcctgaaatttcaacactatcaaaacattctatttttttgaaataatcttcttttaattgcttaacggttacttttcgatttgttttgaagagtaaatcaacaatggctttcttctgttcacctgaaagaaatgctggttttcgcattccttcagtaacatatttgacctttgtcaattcgttataaaccgtaaaatactcataaagcaaactatgttttggtagtactttttcatttggaagatttttatcaaagtttgtcatgcgttcaataaatgattgagctgaagcacctttatcgacaacttcttcaaaattccatggggtaattgtttcttcagacttccgagtcatccatgcaaaacgactattgccacgcgccaatggaccaacataataaggaattcgaaaagtcaagattttttcaatcttctcacgattgtcttttaaaaatggataaaagtcttcttgtcttctcaaaatagcatgcagctcacccaagtgaatttgatggggaatagagccgttgtcaaaggtccgttgcttgcgcagcaaatcttcacgatttagtttcaccaataattcctcagtaccatccattttttctaaaattggtttgataaatttataaaattcttcttggctagctcccccatcaatataacctgcatatccgttttttgattgatcaaaaaagatttctttatacttttctggaagttgttgtcgaactaaagcttttaaaagagtcaagtcttgatgatgttcatcgtagcgtttaatcattgaagctgataggggagccttagttatttcagtatttactcttaggatatctgaaagtaaaatagcatctgataaattcttagctgccaaaaacaaatcagcatattgatctccaatttgcgccaataaattatctaaatcatcatcgtaagtatcttttgaaagctgtaatttagcatcttctgccaaatcaaaatttgatttaaaattaggggtcaaacccaatgacaaagcaatgagattcccaaataagccatttttcttctcaccggggagctgagcaatgagattttctaatcgtcttgatttactcaatcgtgcagaaagaatcgctttagcatctactccacttgcgttaatagggttttcttcaaataattgattgtaggtttgtaccaactggataaatagtttgtccacatcactattatcaggatttaaatctccctcaatcaaaaaatgaccacgaaacttaatcatatgcgctaaggccaaatagattaagcgcaaatccgctttatcagtagaatctaccaatttttttcgcagatgatagatagttggatatttctcatgataagcaacttcatctactatatttccaaaaataggatgacgttcatgcttcttgtcttcttccaccaaaaaagactcttcaagtcgatgaaagaaactatcatctactttcgccatctcatttgaaaaaatctcctgtagataacaaatacgattcttccgacgtgtataccttctacgagctgtccgtttgagacgagtcgcttccgctgtctctccactgtcaaataaaagagcccctataagattttttttgatactgtggcggtctgtatttcccagaaccttgaactttttagacggaaccttatattcatcagtgatcaccgcccatccgacgctatttgtgccgatatctaagcctattgagtatttcttatccatagatcctttctcctctttagatctgctagcataatacctagggctgagctagccgtaaatgaggtttcagcaaaaaacccctcaagacccgtttagaggccccaaggggttatgctagttattgctcagcggattaattattagaaaaattcatccagcatcagatgaaattgcagtttgttcatatccggattatcaatgccatatttctgaaacagacgtttttgcaggctcgggctaaattcgcccaggcagttccacagaatggccagatcctgataacgatccgcaatgcccacacggcccacatcaatgcagccaatcagtttgccttcatcgaaaatcaggttatccaggctaaaatcgccgtgggtcaccacgctatccgggctaaacggcagcagtttatgcatttctttccacacctgttccaccggccagccgttacgttcatcatcaaaatcgctcgcatccaccaggccgttgttcatacggctctgcgcctgggccagacgaaacacacgatcgctgttaaacgggcagttgcacaccggaatgctatgcagacgacgcagaaacacggccagcgcatccacaatgttttcgccgctatccggatattcttccagcacctgaaacgcggttttgcccggaatcgcggtggtcagcagccacgcatcatccggggtgcgaataaaatgtttaatggtcggcagcggcataaattcggtcagccagttcagacgcaccatttcatcggtcacatcgttcgccacgctgcctttgccatgtttcagaaacagttccggcgcatccggtttgccatacagacgataaatggtcgcgccgctctgacccacgttatcacgcgcccatttatagccatacagatccgcatccatgttgctgttcagacgcggacggctacagctcgtttcacgctgaatatggctcataacaccccttgtattactgtttatgtaagcagacagttttattgttcatgatgatatatttttatcttgtgcaatgtaacatcagagattttgagacacaaatttaaatcgtaattattggggacccctggattctcaccaataaaaaacgcccggcggcaaccgagcgttctgaacaaatccagatggagttctgaggtcattactggatctatcaacaggagtccaagactagtcgccagggttttcccagtcacgacgcggccgcaagcttgcatgcctgcaggtcgactctagaggatccccgggtaccgagctcgaattcgcgcggccgcggcctaggcggcctcctgtgtgaaattgttatccgctttaattaa

SEQ ID NO: 28 - DNA, 7631 bp, Artificial
  Synthetic TAPKn-dCas9-nsp
atctttgacagctagctcagtcctaggtataatactagtgaagagcaccggtgctcttcgttttagagctagaaatagcaagttaaaataaggctagtccgttatcaacttgaaaaagtggcaccgagtcggtgctttttttgaagcttgggcccgaacaaaaaaaaaccccgcccctgacagggcggggttttttttgtttgtcggtgaactggatccttaccagctgggcgcgccccccctacgggcttgctctccgggcttcgccctgcgcggtcgctgcgctcccttgccagcccgtggatatgtggacgatggccgcgagcggccaccggctggctcgcttcgctcggcccgtggacaaccctgctggacaagctgatggacaggctgcgcctgcccacgagcttgaccacagggattgcccaccggctacccagccttcgaccacatacccaccggctccaactgcgcggcctgcggccttgccccatcaatttttttaattttctctggggaaaagcctccggcctgcggcctgcgcgcttcgcttgccggttggacaccaagtggaaggcgggtcaaggctcgcgcagcgaccgcgcagcggcttggccttgacgcgcctggaacgacccaagcctatgcgagtgggggcagtcgaagggcgaagcccgcccgcctgccccccgagcctcacggcggcgagtgcgggggttccaagggggcagcgccaccttgggcaaggccgaaggccgcgcagtcgatcaacaagccccggaggggccactttttgccggagggggagccgcgccgaaggcgtgggggaaccccgcaggggtgcccttctttgggcaccaaagaactagatatagggcgaaatgcgaaagacttaaaaatcaacaacttaaaaaaggggggtacgcaacagctcattgcggcaccccccgcaatagctcattgcgtaggttaaagaaaatctgtaattgactgccacttttacgcaacgcataattgttgtcgcgctgccgaaaagttgcagctgattgcgcatggtgccgcaaccgtgcggcacccctaccgcatggagataagcatggccacgcagtccagagaaatcggcattcaagccaagaacaagcccggtcactgggtgcaaacggaacgcaaagcgcatgaggcgtgggccgggcttattgcgaggaaacccacggcggcaatgctgctgcatcacctcgtggcgcagatgggccaccagaacgccgtggtggtcagccagaagacactttccaagctcatcggacgttctttgcggacggtccaatacgcagtcaaggacttggtggccgagcgctggatctccgtcgtgaagctcaacggccccggcaccgtgtcggcctacgtggtcaatgaccgcgtggcgtggggccagccccgcgaccagttgcgcctgtcggtgttcagtgccgccgtggtggttgatcacgacgaccaggacgaatcgctgttggggcatggcgacctgcgccgcatcccgaccctgtatccgggcgagcagcaactaccgaccggccccggcgaggagccgcccagccagcccggcattccgggcatggaaccagacctgccagccttgaccgaaacggaggaatgggaacggcgcgggcagcagcgcctgccgatgcccgatgagccgtgttttctggacgatggcgagccgttggagccgccgacacgggtaacgctgccgcgccggtagggcatgcaaacagggacgcaccgctagcagcgcccctagcggtatcctataaaaaaacacaccgcgccgctagcagcacccctaatataaaataatgttttttataaaaatagtcagtaccacccctacaaaacggtgtcggcgcgttgttgtagccgcgccgacaccgcttttttaaatatcataaagagagtaagagaaactaatttttcataacactctatttataaagaaaaatcagcaaaaacttgtttttgcgtggggtgtggtgcttttggtggtgagaaccaccaacctgttgagcctttttgtggagcgcgcggaccgcggtccagagagcgttcaccgacaaacaacagataaaacgaaaggcccagtctttcgactgagcctttcgttttatttgatgcctggagatccttactcgagttagtcacctcctagctgactcaaatcaatgcgtgtttcataaagaccagtgatggattgatggataagagtggcatctaaaacttcttttgtagacgtatatcgtttacgatcaattgttgtatcaaaatatttaaaagcagcgggagctccaagattcgtcaacgtaaataaatgaataatattttctgcttgttcacgtattggtttgtctctatgtttgttatatgcactaagaactttatctaaattggcatctgctaaaataacacgcttagaaaattcactgatttgctcaataatctcatctaaataatgcttatgctgctccacaaacaattgtttttgttcgttatcttctggactacccttcaacttttcataatgactagctaaatataaaaaattcacatatttgcttggcagagccagctcatttcctttttgtaattctccggcactagccagcatccgtttacgaccgttttctaactcaaaaagactatatttaggtagtttaatgattaagtcttttttaacttccttatatcctttagcttctaaaaagtcaatcggatttttttcaaaggaacttctttccataattgtgatccctagtaactctttaacggattttaacttcttcgatttccctttttccaccttagcaaccactaggactgaataagctaccgttggactatcaaaaccaccatatttttttggatcccagtcttttttacgagcaataagcttgtccgaatttctttttggtaaaattgactccttggagaatccgcctgtctgtacttctgttttcttgacaatattgacttggggcatggacaatactttgcgcactgtggcaaaatctcgccctttatcccagacaatttctccagtttccccattagtttcgattagagggcgtttgcgaatctctccatttgcaagtgtaatttctgttttgaagaagttcatgatattagagtaaaagaaatattttgcggttgctttgcctatttcttgctcagacttagcaatcattttacgaacatcataaactttataatcaccatagacaaactccgattcaagttttggatatttcttaatcaaagcagttccaacgacggcatttagatacgcatcatgggcatgatggtaattgttaatctcacgtactttatagaattggaaatcttttcggaagtcagaaactaatttagattttaaggtaatcactttaacctctcgaataagtttatcattttcatcgtatttagtattcatgcgactatccaaaatttgtgccacatgcttagtgatttggcgagtttcaaccaattggcgtttgataaaaccagctttatcaagttcactcaaacctccacgttcagctttcgttaaattatcaaacttacgttgagtgattaacttggcgtttagaagttgtctccaatagtttttcatctttttgactacttcttcacttggaacgttatccgatttaccacgatttttatcagaacgcgttaagaccttattgtctattgaatcgtctttaaggaaactttgtggaacaatggcatcgacatcataatcacttaaacgattaatatctaattcttggtccacatacatgtctcttccattttggagataatagagatagagcttttcattttgcaattgagtattttcaacaggatgctctttaagaatctgacttcctaattctttgataccttcttcgattcgtttcatacgctctcgcgaatttttctggcccttttgagttgtctgattttcacgtgccatttcaataacgatattttctggcttatgccgccccattactttgaccaattcatcaacaacttttacagtctgtaaaataccttttttaatagcagggctaccagctaaatttgcaatatgttcatgtaaactatcgccttgtccagacacttgtgctttttgaatgtcttctttaaatgtcaaactatcatcatggatcagctgcataaaattgcgattggcaaaaccatctgatttcaaaaaatctaatattgttttgccagattgcttatccctaataccattaatcaattttcgagacaaacgtccccaaccagtataacggcgacgtttaagctgtttcatcaccttatcatcaaagaggtgagcatatgttttaagtctttcctcaatcatctccctatcttcaaataaggtcaatgttaaaacaatatcctctaagatatcttcattttcttcattatccaaaaaatctttatctttaataatttttagcaaatcatggtaggtacctaatgaagcattaaatctatcttcaactcctgaaatttcaacactatcaaaacattctatttttttgaaataatcttcttttaattgcttaacggttacttttcgatttgttttgaagagtaaatcaacaatggctttcttctgttcacctgaaagaaatgctggttttcgcattccttcagtaacatatttgacctttgtcaattcgttataaaccgtaaaatactcataaagcaaactatgttttggtagtactttttcatttggaagatttttatcaaagtttgtcatgcgttcaataaatgattgagctgaagcacctttatcgacaacttcttcaaaattccatggggtaattgtttcttcagacttccgagtcatccatgcaaaacgactattgccacgcgccaatggaccaacataataaggaattcgaaaagtcaagattttttcaatcttctcacgattgtcttttaaaaatggataaaagtcttcttgtcttctcaaaatagcatgcagctcacccaagtgaatttgatggggaatagagccgttgtcaaaggtccgttgcttgcgcagcaaatcttcacgatttagtttcaccaataattcctcagtaccatccattttttctaaaattggtttgataaatttataaaattcttcttggctagctcccccatcaatataacctgcatatccgttttttgattgatcaaaaaagatttctttatacttttctggaagttgttgtcgaactaaagcttttaaaagagtcaagtcttgatgatgttcatcgtagcgtttaatcattgaagctgataggggagccttagttatttcagtatttactcttaggatatctgaaagtaaaatagcatctgataaattcttagctgccaaaaacaaatcagcatattgatctccaatttgcgccaataaattatctaaatcatcatcgtaagtatcttttgaaagctgtaatttagcatcttctgccaaatcaaaatttgatttaaaattaggggtcaaacccaatgacaaagcaatgagattcccaaataagccatttttcttctcaccggggagctgagcaatgagattttctaatcgtcttgatttactcaatcgtgcagaaagaatcgctttagcatctactccacttgcgttaatagggttttcttcaaataattgattgtaggtttgtaccaactggataaatagtttgtccacatcactattatcaggatttaaatctccctcaatcaaaaaatgaccacgaaacttaatcatatgcgctaaggccaaatagattaagcgcaaatccgctttatcagtagaatctaccaatttttttcgcagatgatagatagttggatatttctcatgataagcaacttcatctactatatttccaaaaataggatgacgttcatgcttcttgtcttcttccaccaaaaaagactcttcaagtcgatgaaagaaactatcatctactttcgccatctcatttgaaaaaatctcctgtagataacaaatacgattcttccgacgtgtataccttctacgagctgtccgtttgagacgagtcgcttccgctgtctctccactgtcaaataaaagagcccctataagattttttttgatactgtggcggtctgtatttcccagaaccttgaactttttagacggaaccttatattcatcagtgatcaccgcccatccgacgctatttgtgccgatagctaagcctattgagtatttcttatccatagatcctttctcctctttagatctgctagcataatacctagggctgagctagccgtaaatgaggtttcagcaaaaaacccctcaagacccgtttagaggccccaaggggttatgctagttattgctcagcggattaattattagaaaaattcatccagcatcagatgaaattgcagtttgttcatatccggattatcaatgccatatttctgaaacagacgtttttgcaggctcgggctaaattcgcccaggcagttccacagaatggccagatcctgataacgatccgcaatgcccacacggcccacatcaatgcagccaatcagtttgccttcatcgaaaatcaggttatccaggctaaaatcgccgtgggtcaccacgctatccgggctaaacggcagcagtttatgcatttctttccacacctgttccaccggccagccgttacgttcatcatcaaaatcgctcgcatccaccaggccgttgttcatacggctctgcgcctgggccagacgaaacacacgatcgctgttaaacgggcagttgcacaccggaatgctatgcagacgacgcagaaacacggccagcgcatccacaatgttttcgccgctatccggatattcttccagcacctgaaacgcggttttgcccggaatcgcggtggtcagcagccacgcatcatccggggtgcgaataaaatgtttaatggtcggcagcggcataaattcggtcagccagttcagacgcaccatttcatcggtcacatcgttcgccacgctgcctttgccatgtttcagaaacagttccggcgcatccggtttgccatacagacgataaatggtcgcgccgctctgacccacgttatcacgcgcccatttatagccatacagatccgcatccatgttgctgttcagacgcggacggctacagctcgtttcacgctgaatatggctcataacaccccttgtattactgtttatgtaagcagacagttttattgttcatgatgatatatttttatcttgtgcaatgtaacatcagagattttgagacacaaatttaaatcgtaattattggggacccctggattctcaccaataaaaaacgcccggcggcaaccgagcgttctgaacaaatccagatggagttctgaggtcattactggatctatcaacaggagtccaagactagtcgccagggttttcccagtcacgacgcggccgcaagcttgcatgcctgcaggtcgactctagaggatccccgggtaccgagctcgaattcgcgcggccgcggcctaggcggcctcctgtgtgaaattgttatccgctttaattaa

(19) United States
(12) Patent Application Publication
(10) Pub. No.: US 2024/0016855 A1
(43) Pub. Date: Jan. 18, 2024

(54) TARGETED-ANTIBACTERIAL-PLASMIDS COMBINING CONJUGATION AND CRISPR/CAS SYSTEMS AND USES THEREOF

(71) Applicants: CENTRE NATIONAL DE LA RECHERCHE SCIENTIFIQUE (CNRS), Paris (FR); UNIVERSITE CLAUDE BERNARD - LYON 1, Villeurbanne (FR)

(72) Inventors: Christian LESTERLIN, Lyon Cedex 07 (FR); Sarah BIGOT, Lyon Cedex 07 (FR)

(21) Appl. No.: 18/031,249

(22) PCT Filed: Oct. 12, 2021
(86) PCT No.: PCT/EP2021/078161

§ 371 Date: Apr. 11, 2023

(30) Foreign Application Priority Data
Oct. 13, 2020 (EP) 20306200.5

(51) Int. Cl. A61K 35/74; C12N 15/11; C12N 9/22; C12N 15/70; C12N 1/20

(52) U.S. Cl. CPC A61K 35/74; C12N 15/11; C12N 9/22; C12N 15/70; C12N 1/20; C12N 2310/20; C12N 2800/80; C12N 2800/101

(57) ABSTRACT

The global emergence of drug-resistant bacteria leads to the loss of efficacy of our antibiotics arsenal and severely limits the success of currently available treatments. Here, the inventors developed an innovative strategy based on Targeted-Antibacterial-Plasmids (TAPs) that use bacterial conjugation to deliver CRISPR/Cas systems exerting a strain-specific antibacterial activity. TAPs are highly versatile as they can be directed against any specific genomic or plasmid DNA using the custom algorithm (CSTB) that identifies appropriate targeting spacer sequences. The inventors demonstrate TAPs ability to induce strain-selective killing by introducing lethal DSBs into the targeted genomes. TAPs directed against a plasmid-born carbapenem resistance gene efficiently resensitize the strain to the drug. This work represents an essential step towards the development of an alternative to antibiotic treatments, which can be used to eradicate targeted resistant and/or pathogen bacteria without affecting other non-targeted bacterial communities.

### FIELD OF THE INVENTION

The present invention is in the field of bacteriology and medicine.

### BACKGROUND OF THE INVENTION

The worldwide proliferation of drug-resistant bacteria is predicted to cause a dramatic increase in human deaths due to therapeutic failures in the next decades. The constant emergence of bacterial resistances and the current low rate of antibiotic discovery emphasize the need to develop innovative antibacterial strategies that represent a real alternative to the use of antibiotics. Besides, antibiotics generally lack specificity as they target fundamental processes essential to bacterial proliferation. Consequently, antibiotics affect the whole treated bacterial community without discriminating between unwanted and commensal strains, and also select for drug-resistant strains proliferation. Recent reports demonstrate the possibility to achieve specific antimicrobial activity through the use of Clustered Regularly Interspaced Short Palindromic Repeats (CRISPR) and the associated Cas proteins. CRISPR/Cas systems can achieve bacterial killing through the induction of double-stranded breaks (DSBs) to the chromosome by the Cas9 nuclease (Jinek et al 2012, PMID22745249; Gasiunas et al 2012, PMID22949671). The expression of specific genes can also be inhibited through CRISPR interference (CRISPRi), when using the dead catalytic Cas9 enzyme (dCas9) (Qi et al 2013; Bikard et al 2013; PMID23452860 PMID23761437). CRISPR targeting relies on the ˜16-20 nucleotide target-specific guide RNA (gRNA) sequence, which allow the recruitment of the Cas nuclease to the complementary DNA sequence (Jinek et al 2012; Anders et al 2014; PMID22745249 PMID 25079318). CRISPR targeting is highly specific, especially in bacteria where off-target activity is limited. Yet, to be used as practical antibacterial tools, CRISPR/Cas genes need to be delivered to the targeted bacterium. Bacterial DNA conjugation precisely offers the possibility to transfer long DNA segments to a range of bacterial species, (with the transfer specificity depending on the host-range of the considered conjugation system).

### SUMMARY OF THE INVENTION

The present invention is defined by the claims. In particular, the present invention to Targeted-Antibacterial-Plasmids and uses thereof in particular for therapeutic purposes.

### DETAILED DESCRIPTION OF THE INVENTION

The global emergence of drug-resistant bacteria leads to the loss of efficacy of our antibiotics arsenal and severely limits the success of currently available treatments. Here, the inventors developed an innovative strategy based on Targeted-Antibacterial-Plasmids (TAPs) that use bacterial conjugation to deliver CRISPR/Cas systems exerting a strain-specific antibacterial activity. TAPs are highly versatile as they can be directed against any specific genomic or plasmid DNA using the custom algorithm (CSTB) that identifies appropriate targeting spacer sequences. The inventors demonstrate TAPs ability to induce strain-selective killing by introducing lethal DSBs into the targeted genomes. TAPs directed against a plasmid-born carbapenem resistance gene efficiently resensitize the strain to the drug. This work represents an essential step towards the development of an alternative to antibiotic treatments, which can be used to eradicate targeted resistant and/or pathogen bacteria without affecting other non-targeted bacterial communities.

### Main Definitions

As used herein, the terms “polypeptide”, “peptide”, and “protein” are used interchangeably to refer to polymers of amino acids of any length. The terms also encompass an amino acid polymer that has been modified; for example, disulfide bond formation, glycosylation, lipidation, phosphorylation, or conjugation with a labeling component. Polypeptides when discussed in the context of gene therapy refer to the respective intact polypeptide, or any fragment or genetically engineered derivative thereof, which retains the desired biochemical function of the intact protein.

As used herein, the expression “derived from” refers to a process whereby a first component (e.g., a first polypeptide), or information from that first component, is used to isolate, derive or make a different second component (e.g., a second polypeptide that is different from the first one).

As used herein, the term “nucleic acid sequence” refers to a polymeric form of nucleotides of any length, including deoxyribonucleotides or ribonucleotides, or analogs thereof. A nucleic acid sequence may comprise modified nucleotides, such as methylated nucleotides and nucleotide analogs, and may be interrupted by non-nucleotide components. If present, modifications to the nucleotide structure may be imparted before or after assembly of the polymer.

As used herein, the term “plasmid” refers to a self-contained molecule of double-stranded DNA, usually of bacterial origin, that can readily accept additional (foreign) DNA and which can be readily introduced into a suitable host cell. A plasmid vector often contains coding DNA and promoter DNA and has one or more restriction sites suitable for inserting foreign DNA. Promoter DNA and coding DNA may be from the same gene or from different genes, and may be from the same or different organisms. Typically, a plasmid has a size of desired length, suitably of at least about 5 kb, preferably at least about 10 kb, more preferably at least about 20 or 30 kb, even more preferably at least about 40 or 50 kb. The upper size limit of the plasmid can be chosen as desired, suitably is about 300 kb, preferably about 350 or 400 kb. In some embodiments, the upper size limit is even higher, such as 450 or 500 kb.

As used herein, the term “conjugation” refers to the direct transfer of nucleic acid from one prokaryotic cell to another via direct contact between cells. The cell that carries the transferred DNA after conjugation is termed “exoconjugant”, “exconjugant” or “transconjugant”

As used herein, the term “prokaryotic F factor partitioning system” refers to an active positioning process that ensures proper segregation and faithful distribution of daughter F factors at cell division. This partitioning system contains three functionally distinct regions: two of them (sopA and sopB) encode gene products that act in trans, whereas the third region (sopC) functions in cis. As used herein, the “F factor” or “prokaryotic F factor” refers to a fertility factor found in prokaryotes. It is a ˜100 Kb piece of episomal DNA that enables bacteria to mediate conjugation with other bacteria. The F factor “partitioning system” refers to the system that ensures that both daughter cells inherit a copy of the parental plasmid

As used herein, the term “donor bacterial cell” refers to the bacterial cell that comprises the conjugative plasmid that is intended for being transferred into a recipient bacterial cell.

As used herein, the term “recipient bacterial cell” denotes the bacterial cell that finally receives the conjugative plasmid of the donor bacterial cell.

As used herein, the term “probiotic” is meant to designate live microorganisms which, they are integrated in a sufficient amount, exert a positive effect on health, comfort and wellness beyond traditional nutritional effects. Probiotic microorganisms have been defined as “Live microorganisms which when administered in adequate amounts confer a health benefit on the host” (FAO/WHO 2001).

As used herein, the term “viable probiotic cell” means a microorganism which is metabolically active and that is able to colonize the gastro-intestinal tract of the subject.

As used herein, the term “recombinant plasmid” or “synthetic plasmid” refers to an artificially constructed plasmid, combining nucleic acid sequences obtained from different organisms in nature, using molecular biology protocols.

As used herein, the term “conjugative plasmid” refers to a plasmid that is directly transferred from one donor bacterial cell to one recipient bacterial cell via direct contact between the cells. Numerous naturally occurring conjugative plasmids are known, which can transfer associated genes within restricted species (narrow host range) or between many species (broad host range).

As used herein, the term “Targeted-Antibacterial-Plasmid” or “TAP” refers to a synthetic conjugative plasmid that functions as an autonomous unit of DNA replication within a cell, i.e., capable of replication under its own control and that comprises i) an origin of replication, ii) an origin of transfer, iii) a nucleic acid sequence encoding for a nuclease and iv) one or more nucleic acid sequences that encodes for a guide RNA molecule. According to the present invention the Targeted-Antibacterial-Plasmid of the present invention targets the recipient bacterial cells so as to modify its phenotype and/or kill it.

As used herein, the term “copy number” is the number of plasmids in a bacterial cell. This will be understood to describe a characteristic of a recombinant expression construct present in a donor bacterial cell in greater than a single copy per cell. Most plasmids are classified by the terms “multiple copy number,” “low copy number” or “high copy number,” which describes the ratio of plasmid/chromosome molecules.

As used herein, the term “origin of replication” or “oriV” is intended to encompass regions of nucleotides that are necessary for replication of a plasmid.

As used herein, the term “range” or “host range” refers generally to parameters of both the number and diversity of different bacterial species in which a particular plasmid (natural or recombinant) can replicate. Of these two parameters, one skilled in the art would consider diversity of organisms as generally more defining of host range. For instance, if a plasmid replicates in many species of one group, e.g., Enterobacteriaceae, it may be considered to be of narrow host range. By comparison, if a plasmid is reported to replicate in only a few species, but those species are from phylogenetically diverse groups, that plasmid may be considered of broad host range. As discussed herein both types of plasmids will find utility in the present invention.

As used herein, the term “origin of transfer” or “oriT” is intended to encompass regions of nucleotides that are necessary to permit the transfer of the plasmid from one donor cell to a host. The origin of transfer represents the site on the vector where the transfer process is initiated. It is also defined genetically as the region required in cis to the DNA that is to be transferred. Conjugation-specific DNA replication is initiated within the oriT region which also encodes plasmid transfer factors. Typically, the oriT is about to 40-500 bp in length and contains intrinsic bends and direct and inverted repeats that bind the proteins involved in DNA transfer. The oriT consists of three functionally defined domains: a nicking domain, a transfer domain, and a termination domain. The nic site itself, which is a strand- and sequence-specific cleavage site, is cleaved and religated by relaxase. In most cases, relaxase requires auxiliary proteins that direct relaxase to the nic site and ensure the specificity of the reaction. The sequence of the nic sites identified to date reveal four possible sequences represented by IncF, -P, and -Q and certain Gram-positive plasmids such as pMV158. In addition, there is usually a protein that binds to multiple sites within oriT forming a higher-order structure in the DNA, which is essential for the process. This protein also appears to have a function in anchoring the relaxosome to the transport machinery.

As used herein, the term “nuclease” includes a protein (i.e. an enzyme) that induces a break in a nucleic acid sequence, e.g., a single or a double strand break in a double-stranded DNA sequence.

As used herein, the term “CRISPR/Cas nuclease” has its general meaning in the art and refers to segments of prokaryotic DNA containing clustered regularly interspaced short palindromic repeats (CRISPR) and associated nucleases encoded by Cas genes. In bacteria the CRISPR/Cas loci encode RNA-guided adaptive immune systems against mobile genetic elements (viruses, transposable elements and conjugative plasmids). Three types of CRISPR systems have been identified. CRISPR clusters contain spacers, the sequences complementary to antecedent mobile elements. CRISPR clusters are transcribed and processed into mature CRISPR (Clustered Regularly Interspaced Short Palindromic Repeats) RNA (crRNA). The CRISPR/Cas nucleases Cas9 and Cpf1 belong to the type II and type V CRISPR/Cas system and have strong endonuclease activity to cut target DNA. Cas9 is guided by a mature crRNA that contains about 20 nucleotides of unique target sequence (called spacer) and a trans-activating small RNA (tracrRNA) that also serves as a guide for ribonuclease III-aided processing of pre-crRNA. The crRNA:tracrRNA duplex directs Cas9 to target DNA via complementary base pairing between the spacer on the crRNA and the complementary sequence (called protospacer) on the target DNA. Cas9 recognizes a trinucleotide (NGG for S. pyogenes Cas9) protospacer adjacent motif (PAM) to specify the cut site (the 3^{rd} or the 4^{th} nucleotide upstream from PAM).

As used herein, the term “Cas9” or “Cas9 nuclease” refers to an RNA-guided nuclease comprising a Cas9 protein, or a fragment thereof (e.g., a protein comprising an active or inactive DNA cleavage domain of Cas9, and/or the gRNA binding domain of Cas9). A Cas9 nuclease is also referred to sometimes as a casn1 nuclease or a CRISPR (clustered regularly interspaced short palindromic repeat)-associated nuclease. CRISPR is an adaptive immune system that provides protection against mobile genetic elements (viruses, transposable elements and conjugative plasmids). CRISPR clusters contain spacers, sequences complementary to antecedent mobile elements, and target invading nucleic acids. CRISPR clusters are transcribed and processed into CRISPR RNA (crRNA). In type II CRISPR systems correct processing of pre-crRNA requires a trans-encoded small RNA (tracrRNA), endogenous ribonuclease 3 (rnc) and a Cas9 protein. The tracrRNA serves as a guide for ribonuclease 3-aided processing of pre-crRNA. Subsequently, Cas9/crRNA/tracrRNA endonucleolytically cleaves linear or circular dsDNA target complementary to the spacer. The target strand not complementary to crRNA is first cut endonucleolytically, then trimmed 3′-5′ exonucleolytically. In nature, DNA-binding and cleavage typically requires protein and both RNAs. However, single guide RNAs (“sgRNA”, or simply “gRNA”) can be engineered so as to incorporate aspects of both the crRNA and tracrRNA into a single RNA species. See, e.g., Jinek M., Chylinski K., Fonfara I., Hauer M., Doudna J. A., Charpentier E. Science 337:816-821(2012), the entire contents of which is hereby incorporated by reference. Cas9 recognizes a short motif in the CRISPR repeat sequences (the PAM or protospacer adjacent motif) to help distinguish self versus non-self. Cas9 nuclease sequences and structures are well known to those of skill in the art (see, e.g., “Complete genome sequence of an M1 strain of Streptococcus pyogenes.” Ferretti et al., J. J., McShan W. M., Ajdic D. J., Savic D. J., Savic G., Lyon K., Primeaux C., Sezate S., Suvorov A. N., Kenton S., Lai H. S., Lin S. P., Qian Y., Jia H. G., Najar F. Z., Ren Q., Zhu H., Song L., White J., Yuan X., Clifton S. W., Roe B. A., McLaughlin R. E., Proc. Natl. Acad. Sci. U.S.A. 98:4658-4663(2001); “CRISPR RNA maturation by trans-encoded small RNA and host factor RNase III.” Deltcheva E., Chylinski K., Sharma C. M., Gonzales K., Chao Y., Pirzada Z. A., Eckert M. R., Vogel J., Charpentier E., Nature 471:602-607(2011); and “A programmable dual-RNA-guided DNA endonuclease in adaptive bacterial immunity.” Jinek M., Chylinski K., Fonfara I., Hauer M., Doudna J. A., Charpentier E. Science 337:816-821(2012), the entire contents of each of which are incorporated herein by reference). Cas9 orthologs have been described in various species, including, but not limited to, S. pyogenes and S. thermophilus. Additional suitable Cas9 nucleases and sequences will be apparent to those of skill in the art based on this disclosure, and such Cas9 nucleases and sequences include Cas9 sequences from the organisms and loci disclosed in Chylinski, Rhun, and Charpentier, “The tracrRNA and Cas9 families of type II CRISPR-Cas immunity systems” (2013) RNA Biology 10:5, 726-737; the entire contents of which are incorporated herein by reference. In some embodiments, the term “Cas9” refers to Cas9 from: Corynebacterium ulcerans (NCBI Refs: NC_015683.1, NC_017317.1); Corynebacterium diphtheria (NCBI Refs: NC_016782.1, NC_016786.1); Spiroplasma syrphidicola (NCBI Ref: NC_021284.1); Prevotella intermedia (NCBI Ref: NC_017861.1); Spiroplasma taiwanense (NCBI Ref: NC_021846.1); Streptococcus iniae (NCBI Ref: NC_021314.1); Belliella baltica (NCBI Ref: NC_018010.1); Psychroflexus torquisl (NCBI Ref: NC_018721.1); Streptococcus thermophilus (NCBI Ref: YP_820832.1); Listeria innocua (NCBI Ref: NP_472073.1); Campylobacter jejuni (NCBI Ref: YP_002344900.1); or Neisseria. meningitidis (NCBI Ref: YP_002342100.1).

As used herein, the term “defective CRISPR/Cas nuclease” or “CRISPR/dCas9” refers to a CRISPR/Cas nuclease having lost at least one nuclease domain.

As used herein, the term “guide RNA molecule” generally refers to an RNA molecule (or a group of RNA molecules collectively) that can bind to a CRISPR protein and target the CRISPR protein to a specific location within a target DNA. A guide RNA can comprise two segments: a DNA-targeting guide segment and a protein-binding segment. The DNA-targeting segment comprises a nucleotide sequence that is complementary to (or at least can hybridize to under stringent conditions) a target sequence. The protein-binding segment interacts with a CRISPR protein, such as a Cas9 or Cas9 related polypeptide. These two segments can be located in the same RNA molecule or in two or more separate RNA molecules. When the two segments are in separate RNA molecules, the molecule comprising the DNA-targeting guide segment is referred to as the CRISPR RNA (“crRNA”), while the molecule comprising the protein-binding segment is referred to as the trans-activating RNA (“tracrRNA”). Typically the crRNA comprises at least one spacer sequence and at least one repeat sequence, or a portion thereof, linked to the 5′ end of the spacer sequence. The design of a crRNA of this invention will vary based on the CRISPR-Cas system in which the crRNA is to be used. The crRNAs of this invention are synthetic, made by man and not found in nature. Typically, a crRNA may comprise, from 5′ to 3′, a repeat sequence (full length or portion thereof (“handle”)), a spacer sequence, and a repeat sequence (full length or portion thereof). In some embodiments, a crRNA may comprise, from 5′ to 3′, a repeat sequence (full length or portion thereof (“handle”)) and a spacer sequence. The tracr nucleic acid comprises from 5′ to 3′ a bulge, a nexus hairpin and terminal hairpins, and optionally, at the 5′ end, an upper stem (See, Briner et al. (2014) Molecular Cell. 56(2):333-339). A tracrRNA functions in hybridizing to the repeat portion of mature or immature crRNAs, recruits Cas9 protein to the target site, and may facilitate the catalytic activity of Cas9 by inducting structural rearrangement. Sequences for tracrRNAs are specific to the CRISPR-Cas Type II system and can be variable. When a phasmid is engineered to comprise a heterologous Type II CRISPR-Cas system in addition to a Type II crRNA, any tracr nucleic acid, known or later identified, can be used. In some embodiments, the tracr nucleic acid is fused to the crRNA of the invention to form a single guide nucleic acid.

As used herein, the term “target nucleic acid sequence,” “target sequence” or “target region,” as used herein, means a specific sequence or the complement thereof that one wishes to bind to using the CRISPR system as disclosed herein. More particularly a “target nucleic acid strand” refers to a strand of a target nucleic acid that is subject to base-pairing with a guide RNA as disclosed herein. That is, the strand of a target nucleic acid that hybridizes with the crRNA and guide sequence is referred to as the “target nucleic acid strand.” The other strand of the target nucleic acid, which is not complementary to the guide sequence, is referred to as the “non-complementary strand.” In the case of double-stranded target nucleic acid (e.g., DNA), each strand can be a “target nucleic acid strand” to design crRNA and guide RNAs and used to practice the method of this invention as long as there is a suitable PAM site.

As used herein, the term “double strand break” or “DSB” refers to two breaks in a nucleic acid molecule, e.g., a DNA molecule: a first break in a first strand of the nucleic acid molecule, and a second break in a second strand of the nucleic acid molecule.

As used herein, the term “promoter” is intended to encompass DNA sequences that mediate transcription of a nucleic acid in a cell.

As used herein, the term “constitutive promoter” is used to describe a promoter that is in a permanent state of activity allowing for gene expression in the absence of any activating biotic or abiotic regulatory factors. Persons skilled in the art are well aware of the meaning of such terms as “strong constitutive promoters” and “weak constitutive promoters”. Nevertheless, the term “weak constitutive promoter” as defined herein refers to a promoter having expression levels that are 10× or less than a strong constitutive promoter. For example, an ubiquitin promoter (with its first intron) is generally known to be a strong constitutive promoter, and it is also known that deletion of the first intron reduces expression of the promoter ten-fold rendering it a weak constructive promoter.

As used herein, the term “operatively linked” is intended to describe the linkage between nucleic acids wherein the position and proximity of the linkage ensures coupled replication and is sufficient and appropriate to be recognized by trans-acting transcription factors and other cellular factors whereby polypeptide-encoding nucleic acid is efficiently expressed under appropriate conditions.

As used herein, the term “complementarity” refers to the ability of a nucleic acid to form hydrogen bond(s) with another nucleic acid sequence by either traditional Watson-Crick base-pairing or other non-traditional types. A percent complementarity indicates the percentage of residues in a nucleic acid molecule which can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary). “Perfectly complementary” means that all the contiguous residues of a nucleic acid sequence will hydrogen bond with the same number of contiguous residues in a second nucleic acid sequence. “Substantially complementary” as used herein refers to a degree of complementarity that is at least 60%, 65%, 70%, 75%, 80%, 85%, 90%, 95%, 97%, 98%, 99%, or 100% over a region of 8, 9, 10, 11, 12, 13, 14, 15, 16, 17, 18, 19, 20, 21, 22, 23, 24, 25, 30, 35, 40, 45, 50, or more nucleotides, or refers to two nucleic acids that hybridize under stringent conditions.

As used herein, the term “restriction enzyme” will be understood to mean any of a group of enzymes, produced by bacteria, which cleave molecules of DNA internally at specific base sequences. Examples of restriction enzymes would include: BspEI; SpaI, BglII; NsiI; NotI; SacI; SpeI; and AlwNI.

As used herein, the term “restriction site” will be understood to mean a sequence of bases in a DNA molecule that is recognized by a restriction enzyme.

As used herein, the term “cloning” refers to the process of ligating a DNA molecule into a plasmid.

As used herein, the term “selecting” refers to the identification and isolation of a cell such as the donor cell or recipient bacterial cell that contains the plasmid of interest (such as the plasmid of the present invention).

As used herein, the term “selection marker gene” refers to a gene that encodes a polypeptide that provides a phenotype to the cell containing the gene such that the phenotype allows either positive or negative, selection of cells containing the selection marker gene. The selection marker gene may be used to distinguish between transformed and non-transformed cells.

As used herein, the term “about” defines a deviation of 20%, preferably of 10%, more preferably of 7% and even more preferably of 3% of a given value.

As used herein, the term “antibiotic” refers to a classical antibiotic that is produced by a microorganism that is antagonistic to the growth of other microorganisms and also encompasses more generally an antimicrobial agent that is capable of killing or inhibiting the growth of a microorganism, including chemically synthesised versions and variants of naturally occurring antibiotics.

As used herein, the term “carbapenem” denotes a class of β-lactam antibiotics in which the sulfur atom in position 1 of the β-lactam structure has been replaced with a carbon atom, and an unsaturation has been introduced, thus having a molecular structure which renders them resistant to most β-lactamases, which have a broad spectrum of antibacterial activity. In view of their resistance to most bacterial β-lactamases, carbapenems are one of the antibiotics of last resort for many bacterial infections, such as Escherichia coli (E. coli) and Klebsiella pneumoniae.

As used herein, the term “antibiotic resistance gene” encompasses a gene that encodes a product or transcribes a functional RNA that confers antibiotic resistance. For example, the antibiotic resistance gene may be a gene or the encoding portion thereof which contributes to any of the four resistance mechanisms described above. The antibiotic resistance gene may for example encode (1) an enzyme which degrades an antibiotic, (2) an enzyme which modifies an antibiotic, (3) a pump such as an efflux pump, or (4) a mutated target which suppresses the effect of the antibiotic.

As used herein, the term “food” refers to liquid (i.e. drink), solid or semi-solid dietetic compositions, especially total food compositions (food-replacement), which do not require additional nutrient intake or food supplement compositions.

As used herein, the term “patient” or “subject” refers to humans or animals (animals being particularly useful as models for clinical efficacy). In some embodiments, the subject can be human or any other animal (e.g., birds and mammals) susceptible to bacterial infection (e.g. domestic animals such as cats and dogs; livestock and farm animals such as horses, cows, pigs, chickens, etc.). Typically said subject is a mammal including a non-primate (e.g., a camel, donkey, zebra, cow, pig, horse, goat, sheep, cat, dog, rat, and mouse) and a primate (e.g., a monkey, chimpanzee, and a human). In some embodiments, the subject is a non-human animal. In some embodiments, the subject is a farm animal or pet. In some embodiments, the subject is a human. In some embodiments, the subject is a human infant. In some embodiments, the subject is a human child. In some embodiments, the subject is a human adult. In some embodiments, the subject is an elderly human. In some embodiments, the subject is a premature human infant.

As used herein, the term “treatment” or “treat” refer to both prophylactic or preventive treatment as well as curative or disease modifying treatment, including treatment of patient at risk of contracting the disease or suspected to have contracted the disease as well as patients who are ill or have been diagnosed as suffering from a disease or medical condition, and includes suppression of clinical relapse. The treatment may be administered to a patient having a medical disorder or who ultimately may acquire the disorder, in order to prevent, cure, delay the onset of, reduce the severity of, or ameliorate one or more symptoms of a disorder or recurring disorder, or in order to prolong the survival of a patient beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a patient during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a patient during treatment of an illness, e.g., to keep the patient in remission for long periods of time (months or years). A maintenance regimen may employ continuous therapy (e.g., administering a drug at a regular interval, e.g., weekly, monthly, yearly, etc.) or intermittent therapy (e.g., interrupted treatment, intermittent treatment, treatment at relapse, or treatment upon achievement of a particular predetermined criteria [e.g., pain, disease manifestation, etc.]).

As used herein, the term “therapeutically effective amount” refers to an amount of a donor bacterial cells that is sufficient to produce a desired effect, which can be a therapeutic and/or beneficial effect. The effective amount will vary with the age, general condition of the subject, the severity of the condition being treated, the particular agent administered, the duration of the treatment, the nature of any concurrent treatment, the pharmaceutically acceptable carrier used, and like factors within the knowledge and expertise of those skilled in the art. As appropriate, an “effective amount” in any individual case can be determined by one of skill in the art by reference to the pertinent texts and literature and/or by using routine experimentation. In some embodiments, an effective amount of a host bacterium and/or composition thereof may be about 10⁵ to about 10¹⁰ colony forming units (CFU). In some embodiments, an effective amount may be an amount that reduces the bacterial recipient cell load by about 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, or 100%, and any value or range therein.

As used herein, the term “pharmaceutical composition” refers to a composition described herein, or pharmaceutically acceptable salts thereof, with other agents such as carriers and/or excipients. The pharmaceutical compositions as provided herewith typically include a pharmaceutically acceptable carrier.

As used herein, the term “pharmaceutically acceptable carrier” includes any and all solvents, diluents, or other liquid vehicle, dispersion or suspension aids, surface active agents, isotonic agents, thickening or emulsifying agents, preservatives, solid binders, lubricants and the like, as suited to the particular dosage form desired. Remington's Pharmaceutical-Sciences, Sixteenth Edition, E. W. Martin (Mack Publishing Co., Easton, Pa., 1980) discloses various carriers used in formulating pharmaceutical compositions and known techniques for the preparation thereof.

Targeted-Antibacterial Plasmids

The first object of the present invention relates to a Targeted-Antibacterial-plasmid (TAP) comprising i) an origin of replication, ii) an origin of transfer, iii) a genetically-engineered nucleic acid sequences encoding for a nuclease and iv) one or more genetically-engineered nucleic acid sequence(s) encoding for a guide RNA molecule.

i) Origin of Replication

In some embodiments, the origin of replication is typically host specific and governs the host range of the plasmid. Examples of broad range (“promiscuous”) plasmids from which origin of replication may be obtained include, but are not limited to: R6K, RK2, p15A and RSF1010. For instance it is expected that the plasmid of the present invention will function in all gram negative bacteria and will be particularly useful in the genera Acetobacter, Acinetobacter, Aeromonas, Agrobacterium, Alcaligenes, Anabaena, Azorizobium, Bartonella, Bordetella, Brucella, Burkholderia, Campylobacter, Caulobacter, Chromatium, Comamonas, Cytophaga, Deinococcus, Erwinia, Erythrobacter, Escherichia, Flavobacterium, Hyphomicrobium, Klebsiella, Methanobacterium, Methylbacterium, Methylobacillus, Methylobacter, Methylobacterium, Methylococcus, Methylocystis, Methylomicrobium, Methylomonas, Methylophilus, Methylosinus, Myxococcus, Pantoea, Paracoccus, Pseudomonas, Rhizobium, Rhodobacter, Salmonella, Shigella, Sphingomonas and Vibrio.

In some embodiments, the origin of replication is the replication origin of one plasmid selected from the group consisting of pEBFP-N1, pECFP-N1, pEGFP-N1, pEGFP-N2, pEGFP-N3, pEYFP-N1, pEBEP-C1, pECFP-C1, pEGFP-C1, pEGFP-C2, pEGFP-C3, pEYFP-C1, pEGFP-F, pCMS-EGFP, pIIRES2-EGFP, pd2ECFP-N1, pd2EGFP-N1, pd2EYFP-N1, pd1EGFP-N1 and sold by Clontech Laboratories Inc. (Palo Alto, Calif); pCMV-Script, pCMV-Tag, pDual, pBK-CMV and pBK-RSV sold by Stratagene (La Jolla, Calif.); and pTARGET®, pCI, and pCI-Neo sold by Promega Corporation (Madison, Wis.) plasmids.

In some embodiments, the origin of replication is the origin of replication of pBBR1 plasmid. In some embodiments, the origin of replication consists of the nucleic acid sequence of SEQ ID NO:1.

>oriV-pBBR1
SEQ ID NO: 1
gcttatctccatgcggtaggggtgccgcacggttgcggcaccatgcgcaa
tcagctgcaacttttcggcagcgcgacaacaattatgcgttgcgtaaaag
tggcagtcaattacagattttctttaacctacgcaatgagctattgcggg
gggtgccgcaatgagctgttgcgtaccccccttttttaagttgttgattt
ttaagtctttcgcatttcgccctatatctagttctttggtgcccaaagaa
gggcacccctgcggggttcccccacgccttcggcgcggctccccctccgg
caaaaagtggcccctccggggcttgttgatcgactgcgcggccttcggcc
ttgcccaaggtggcgctgcccccttggaacccccgcactcgccgccgtga
ggctcggggggcaggcgggcgggcttcgcccttcgactgcccccactcgc
ataggcttgggtcgttccaggcgcgtcaaggccaagccgctgcgcggtcg
ctgcgcgagccttgacccgccttccacttggtgtccaaccggcaagcgaa
gcgcgcaggccgcaggccggaggcttttccccagagaaaattaaaaaaat
tgatggggcaaggccgcaggccgcgcagttggagccggtgggtatgtggt
cgaaggctgggtagccggtgggcaatccctgtggtcaagctcgtgggcag
gcgcagcctgtccatcagcttgtccagcagggttgtccacgggccgagcg
aagcgagccagccggtggccgc

ii) Origin of Transfer

In some embodiments, the origin of transfer derives from of the promiscuous IncP plasmids RP4 and R751. In some embodiments, the origin of transfer is the oriT of the RP4 plasmid. In some embodiments, the origin of transfer of the present invention consists of the nucleic acid sequence of SEQ ID NO:2.

>oriT-RK2
SEQ ID NO: 2
gggcaggataggtgaagtaggcccacccgcgagcgggtgttccttcttca
ctgtcccttattcgcacctggcggtgctcaacgggaatcctgctctgcga
ggctggccg

In some embodiments, the origin of transfer is the oriTF of the F plasmid to render the plasmid mobilizable by the transfer proteins Tra from the conjugative F plasmi contained in a donor bacterial cell. In some embodiments, the origin of transfer of the present invention consists of the nucleic acid sequence of SEQ ID NO:3.

oriT-F
SEQ ID NO: 3
aggctcaacaggttggtggttctcaccaccaaaagcaccacaccccacgc
aaaaacaagtttttgctgatttttctttataaatagagtgttatgaaaaa
ttagtttctcttactctctttatgatatttaaaaaagcggtgtcggcgcg
gctacaacaacgcgccgacaccgttttgtaggggtggtactgactatttt
tataaaaaacattattttatattaggggtgctgctagcggcgcggtgtgt
ttttttataggataccgctaggggcgctgctagcggtgcg

iii) Nuclease

In some embodiments, the plasmid of the present invention encodes for a CRISPR-associated endonuclease. Various CRISPR/Cas nucleases can be used in this invention. Non-limiting examples of suitable CRISPR/CRISPR/Cas nucleases include Cas3, Cas4, Cas5, Cas5e (or CasD), Cas6, Cas6e, Cas6f, Cas7, Cas8a1, Cas8a2, Cas8b, Cas8c, Cas9, Cas10, Cas10d, CasF, CasG, CasH, Csy1, Csy2, Csy3, Cse1 (or CasA), Cse2 (or CasB), Cse3 (or CasE), Cse4 (or CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csz1, Csx15, Csf1, Csf2, Csf3, Csf4, and Cu1966. See e.g., WO2014144761 WO2014144592, WO2013176772, US20140273226, and US20140273233, the contents of which are incorporated herein by reference in their entireties.

In some embodiments, the CRISPR/Cas nuclease is derived from a Cas9 protein. The Cas9 protein can be from Streptococcus pyogenes, Streptococcus thermophilus, Streptococcus sp., Nocardiopsis dassonvillei, Streptomyces pristinaespiralis, Streptomyces viridochromogenes, Streptomyces viridochromogenes, Streptosporangium roseum, Streptosporangium roseum, Alicyclobacillus acidocaldarius, Bacillus pseudomycoides, Bacillus selenitireducens, Exiguobacterium sibiricum, Lactobacillus delbrueckii, Lactobacillus salivarius, Microscilla marina, Burkholderiales bacterium, Polaromonas naphthalenivorans, Polaromonas sp., Crocosphaera watsonii, Cyanothece sp., Microcystis aeruginosa, Synechococcus sp., Acetohalobium arabaticum, Ammonfex degensii, Caldicelulosiruptor becscii, Candidatus Desulforudis, Clostridium botulinum, Clostridium difficile, Finegoldia magna, Natranaerobius thermophilus, Pelotomaculum thermopropionicum, Acidithiobacillus caldus, Acidithiobacillus ferrooxidans, Allochromatium vinosum, Marinobacter sp., Nitrosococcus halophilus, Nitrosococcus watsoni, Pseudoalteromonas haloplanktis, Ktedonobacter racemifer, Methanohalobium evestigatum, Anabaena variabilis, Nodularia spumigena, Nostoc sp., Arthrospira maxima, Arthrospira platensis, Arthrospira sp., Lyngbya sp., Microcoleus chthonoplastes, Oscillatoria sp., Petrotoga mobilis, Thermosipho africanus, or Acaryochloris marina, inter alia.

In some embodiments, the Cas9 nuclease can have a nucleotide sequence identical to the wild type Streptococcus pyogenes sequence. In some embodiments, the Cas9 nuclease comprises the amino acid sequence as set forth in SEQ ID NO: 4.

>Cas9 sequence
SEQ ID NO: 4
MDKKYSIGLAIGTNSVGWAVITDEYKVPSKKFKVLGNTDRHSIKKNLIGA
LLFDSGETAEATRLKRTARRRYTRRKNRICYLQEIFSNEMAKVDDSFFHR
LEESFLVEEDKKHERHPIFGNIVDEVAYHEKYPTIYHLRKKLVDSTDKAD
LRLIYLALAHMIKFRGHFLIEGDLNPDNSDVDKLFIQLVQTYNQLFEENP
INASGVDAKAILSARLSKSRRLENLIAQLPGEKKNGLFGNLIALSLGLTP
NFKSNEDLAEDAKLQLSKDTYDDDLDNLLAQIGDQYADLFLAAKNLSDAI
LLSDILRVNTEITKAPLSASMIKRYDEHHQDLTLLKALVRQQLPEKYKEI
FFDQSKNGYAGYIDGGASQEEFYKFIKPILEKMDGTEELLVKLNREDLLR
KQRTEDNGSIPHQIHLGELHAILRRQEDFYPFLKDNREKIEKILTFRIPY
YVGPLARGNSRFAWMTRKSEETITPWNFEEVVDKGASAQSFIERMTNEDK
NLPNEKVLPKHSLLYEYFTVYNELTKVKYVTEGMRKPAFLSGEQKKAIVD
LLFKTNRKVTVKQLKEDYFKKIECFDSVEISGVEDRFNASLGTYHDLLKI
IKDKDELDNEENEDILEDIVLTLTLFEDREMIEERLKTYAHLEDDKVMKQ
LKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKV
MGRHKPENIVIEMARENQTTQKGQKNSRERMKRIEEGIKELGSQILKEHP
VENTQLQNEKLYLYYLQNGRDMYVDQELDINRLSDYDVDHIVPQSFLKDD
SIDNKVLTRSDKNRGKSDNVPSEEVVKKMKNYWRQLLNAKLITQRKFDNL
TKAERGGLSELDKAGFIKRQLVETRQITKHVAQILDSRMNTKYDENDKLI
REVKVITLKSKLVSDFRKDFQFYKVREINNYHHAHDAYLNAVVGTALIKK
YPKLESEFVYGDYKVYDVRKMIAKSEQEIGKATAKYFFYSNIMNFFKTEI
TLANGEIRKRPLIETNGETGEIVWDKGRDFATVRKVLSMPQVNIVKKTEV
QTGGFSKESILPKRNSDKLIARKKDWDPKKYGGFDSPTVAYSVLVVAKVE
KGKSKKLKSVKELLGITIMERSSFEKNPIDFLEAKGYKEVKKDLIIKLPK
YSLFELENGRKRMLASAGELQKGNELALPSKYVNFLYLASHYEKLKGSPE
DNEQKQLFVEQHKHYLDEIIEQISEFSKRVILADANLDKVLSAYNKHRDK
PIREQAENIIHLFTLTNLGAPAAFKYFDTTIDRKRYTSTKEVLDATLIHQ
SITGLYETRIDLSQLGGD

In some embodiments, the CRISPR-associated endonuclease can be a sequence from other species, for example other Streptococcus species, such as thermophilus; Pseudomonas aeruginosa, Escherichia coli, or other sequenced bacteria genomes and archaea, or other prokaryotic microorganisms. Alternatively, the wild type Streptococcus pyogenes Cas9 sequence can be modified. Alternatively, the Cas9 nuclease sequence can be for example, the sequence contained within a commercially available vector such as pX330, pX260 or pMJ920 from Addgene (Cambridge, MA). In some embodiments, the Cas9 endonuclease can have an amino acid sequence that is a variant or a fragment of any of the Cas9 endonuclease sequences of Genbank accession numbers KM099231.1 GL669193757; KM099232.1; GL669193761; or KM099233.1 GL669193765 or Cas9 amino acid sequence of pX330, pX260 or pMJ920 (Addgene, Cambridge, MA).

In some embodiments, the CRISPR/Cas nuclease consists of a mutant CRISPR/Cas nuclease i.e. a protein having one or more point mutations, insertions, deletions, truncations, a fusion protein, or a combination thereof. In some embodiments, the mutant has the RNA-guided DNA binding activity, but lacks one or both of its nuclease active sites. In some embodiments, the mutant comprises an amino acid sequence having at least 50% of identity with the wild type amino acid sequence of the CRISPR/Cas nuclease.

In some embodiments, the CRISPR/Cas nuclease is a mutant of a wild type CRISPR/Cas nuclease (such as Cas9) or a fragment thereof. In some embodiments, the CRISPR/Cas nuclease is a mutant Cas9 protein from S. pyogenes. In some embodiments, the CRISPR/Cas nuclease of the present invention is a defective Cas9, i.e. the Cas9 from S. pyrogenes having at least one mutation selected from the group consisting of D10A and H840A. In some embodiments, the base-editing enzyme of the present invention comprises a defective CRISPR/Cas nuclease. The sequence recognition mechanism is the same as for the non-defective CRISPR/Cas nuclease. Typically, the defective CRISPR/Cas nuclease of the invention comprises at least one RNA binding domain. The RNA binding domain interacts with a guide RNA molecule as defined hereinafter. However the defective CRISPR/Cas nuclease of the invention is a modified version with no nuclease activity. Accordingly, the defective CRISPR/Cas nuclease specifically recognizes the guide RNA molecule and thus guides the base-editing enzyme to its target DNA sequence.

In some embodiments, the CRISPR/Cas nuclease of the present invention is nickase and more particularly a Cas9 nickase i.e. the Cas9 from S. pyogenes having one mutation selected from the group consisting of D10A and H840A. In some embodiments, the nickase of the present invention comprises the amino acid sequence as set forth in SEQ ID NO:5 or SEQ ID NO:6.

>S. pyogenes nCas9 Protein Sequence having
the D10A mutation
SEQ ID NO: 5
MDKKYSIGLAIGTNSVGWAVITDEYKVPSKKFKVLGNTDRHSIKKNLIGA
LLFDSGETAEATRLKRTARRRYTRRKNRICYLQEIFSNEMAKVDDSFFHR
LEESFLVEEDKKHERHPIFGNIVDEVAYHEKYPTIYHLRKKLVDSTDKAD
LRLIYLALAHMIKERGHFLIEGDLNPDNSDVDKLFIQLVQTYNQLFEENP
INASGVDAKAILSARLSKSRRLENLIAQLPGEKKNGLFGNLIALSLGLTP
NFKSNFDLAEDAKLQLSKDTYDDDLDNLLAQIGDQYADLFLAAKNLSDAI
LLSDILRVNTEITKAPLSASMIKRYDEHHQDLTLLKALVRQQLPEKYKEI
FFDQSKNGYAGYIDGGASQEEFYKFIKPILEKMDGTEELLVKLNREDLLR
KQRTFDNGSIPHQIHLGELHAILRRQEDFYPFLKDNREKIEKILTFRIPY
YVGPLARGNSRFAWMTRKSEETITPWNFEEVVDKGASAQSFIERMTNFDK
NLPNEKVLPKHSLLYEYFTVYNELTKVKYVTEGMRKPAFLSGEQKKAIVD
LLFKTNRKVTVKQLKEDYFKKIECFDSVEISGVEDRFNASLGTYHDLLKI
IKDKDELDNEENEDILEDIVLTLTLFEDREMIEERLKTYAHLEDDKVMKQ
LKRRRYTGWGRLSRKLINGIRDKQSGKTILDELKSDGFANRNFMQLIHDD
SLTFKEDIQKAQVSGQGDSLHEHIANLAGSPAIKKGILQTVKVVDELVKV
MGRHKPENIVIEMARENQTTQKGQKNSRERMKRIEEGIKELGSQILKEHP
VENTQLQNEKLYLYYLQNGRDMYVDQELDINRLSDYDVDHIVPQSFLKDD
SIDNKVLTRSDKNRGKSDNVPSEEVVKKMKNYWRQLLNAKLITQRKFDNL
TKAERGGLSELDKAGFIKRQLVETRQITKHVAQILDSRMNTKYDENDKLI
REVKVITLKSKLVSDERKDFQFYKVREINNYHHAHDAYLNAVVGTALIKK
YPKLESEFVYGDYKVYDVRKMIAKSEQEIGKATAKYFFYSNIMNFFKTEI
TLANGEIRKRPLIETNGETGEIVWDKGRDFATVRKVLSMPQVNIVKKTEV
QTGGFSKESILPKRNSDKLIARKKDWDPKKYGGFDSPTVAYSVLVVAKVE
KGKSKKLKSVKELLGITIMERSSFEKNPIDFLEAKGYKEVKKDLIIKLPK
YSLFELENGRKRMLASAGELQKGNELALPSKYVNFLYLASHYEKLKGSPE
DNEQKQLFVEQHKHYLDEIIEQISEFSKRVILADANLDKVLSAYNKHRDK
PIREQAENIIHLFTLTNLGAPAAFKYFDTTIDRKRYTSTKEVLDATLIHQ
SITGLYETRIDLSQLGGD
>S. pyogenes nCas9 Protein Sequence having
the H840A mutation
SEQ ID NO: 6
MDKKYSIGLDIGTNSVGWAVITDEYKVPSKKFKVLGNTDRHSIKKNLIGA
LLFDSGETAEATRLKRTARRRYTRRKNRICYLQEIFSNEMAKVDDSFFHR
LEESFLVEEDKKHERHPIFGNIVDEVAYHEKYPTIYHLRKKLVDSTDKAD
LRLIYLALAHMIKFRGHFLIEGDLNPDNSDVDKLFIQLVQTYNQLFEENP
INASGVDAKAILSARLSKSRRLENLIAQLPGEKKNGLFGNLIALSLGLTP
NFKSNFDLAEDAKLQLSKDTYDDDLDNLLAQIGDQYADLFLAAKNLSDAI
LLSDILRVNTEITKAPLSASMIKRYDEHHQDLTLLKALVRQQLPEKYKEI
FFDQSKNGYAGYIDGGASQEEFYKFIKPILEKMDGTEELLVKLNREDLLR
KQRTFDNGSIPHQIHLGELHAILRRQEDFYPFLKDNREKIEKILTFRIPY
YVGPLARGNSRFAWMTRKSEETITPWNFEEVVDKGASAQSFIERMTNFDK
NLPNEKVLPKHSLLYEYFTVYNELTKVKYVTEGMRKPAFLSGEQKKAIVD
LLFKTNRKVTVKQLKEDYFKKIECFDSVEISGVEDRFNASLGTYHDLLKI
IKDKDELDNEENEDILEDIVLTLTLFEDREMIEERLKTYAHLEDDKVMKQ
LKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKV
MGRHKPENIVIEMARENQTTQKGQKNSRERMKRIEEGIKELGSQILKEHP
VENTQLQNEKLYLYYLQNGRDMYVDQELDINRLSDYDVDAIVPQSFLKDD
SIDNKVLTRSDKNRGKSDNVPSEEVVKKMKNYWRQLLNAKLITQRKFDNL
TKAERGGLSELDKAGFIKRQLVETRQITKHVAQILDSRMNTKYDENDKLI
REVKVITLKSKLVSDFRKDFQFYKVREINNYHHAHDAYLNAVVGTALIKK
YPKLESEFVYGDYKVYDVRKMIAKSEQEIGKATAKYFFYSNIMNFFKTEI
TLANGEIRKRPLIETNGETGEIVWDKGRDFATVRKVLSMPQVNIVKKTEV
QTGGFSKESILPKRNSDKLIARKKDWDPKKYGGFDSPTVAYSVLVVAKVE
KGKSKKLKSVKELLGITIMERSSFEKNPIDFLEAKGYKEVKKDLIIKLPK
YSLFELENGRKRMLASAGELQKGNELALPSKYVNFLYLASHYEKLKGSPE
DNEQKQLFVEQHKHYLDEIIEQISEFSKRVILADANLDKVLSAYNKHRDK
PIREQAENIIHLFTLTNLGAPAAFKYFDTTIDRKRYTSTKEVLDATLIHQ
SITGLYETRIDLSQLGGD

In some embodiments, the nucleic acid sequence encoding for the Cas9 protein consists of the nucleic acid sequence of SEQ ID NO:7.

>Cas9 (addgene #44250)
SEQ ID NO: 7
atggataagaaatactcaataggcttagatatcggcacaaatagcgtcgg
atgggcggtgatcactgatgaatataaggttccgtctaaaaagttcaagg
ttctgggaaatacagaccgccacagtatcaaaaaaaatcttataggggct
cttttatttgacagtggagagacagcggaagcgactcgtctcaaacggac
agctcgtagaaggtatacacgtcggaagaatcgtatttgttatctacagg
agattttttcaaatgagatggcgaaagtagatgatagtttctttcatcga
cttgaagagtcttttttggtggaagaagacaagaagcatgaacgtcatcc
tatttttggaaatatagtagatgaagttgcttatcatgagaaatatccaa
ctatctatcatctgcgaaaaaaattggtagattctactgataaagcggat
ttgcgcttaatctatttggccttagcgcatatgattaagtttcgtggtca
ttttttgattgagggagatttaaatcctgataatagtgatgtggacaaac
tatttatccagttggtacaaacctacaatcaattatttgaagaaaaccct
attaacgcaagtggagtagatgctaaagcgattctttctgcacgattgag
taaatcaagacgattagaaaatctcattgctcagctccccggtgagaaga
aaaatggcttatttgggaatctcattgctttgtcattgggtttgacccct
aattttaaatcaaattttgatttggcagaagatgctaaattacagctttc
aaaagatacttacgatgatgatttagataatttattggcgcaaattggag
atcaatatgctgatttgtttttggcagctaagaatttatcagatgctatt
ttactttcagatatcctaagagtaaatactgaaataactaaggctcccct
atcagcttcaatgattaaacgctacgatgaacatcatcaagacttgactc
ttttaaaagctttagttcgacaacaacttccagaaaagtataaagaaatc
ttttttgatcaatcaaaaaacggatatgcaggttatattgatgggggagc
tagccaagaagaattttataaatttatcaaaccaattttagaaaaaatgg
atggtactgaggaattattggtgaaactaaatcgtgaagatttgctgcgc
aagcaacggacctttgacaacggctctattccccatcaaattcacttggg
tgagctgcatgctattttgagaagacaagaagacttttatccatttttaa
aagacaatcgtgagaagattgaaaaaatcttgacttttcgaattccttat
tatgttggtccattggcgcgtggcaatagtcgttttgcatggatgactcg
gaagtctgaagaaacaattaccccatggaattttgaagaagttgtcgata
aaggtgcttcagctcaatcatttattgaacgcatgacaaactttgataaa
aatcttccaaatgaaaaagtactaccaaaacatagtttgctttatgagta
ttttacggtttataacgaattgacaaaggtcaaatatgttactgaaggaa
tgcgaaaaccagcatttctttcaggtgaacagaagaaagccattgttgat
ttactcttcaaaacaaatcgaaaagtaaccgttaagcaattaaaagaaga
ttatttcaaaaaaatagaatgttttgatagtgttgaaatttcaggagttg
aagatagatttaatgcttcattaggtacctaccatgatttgctaaaaatt
attaaagataaagattttttggataatgaagaaaatgaagatatcttaga
ggatattgttttaacattgaccttatttgaagatagggagatgattgagg
aaagacttaaaacatatgctcacctctttgatgataaggtgatgaaacag
cttaaacgtcgccgttatactggttggggacgtttgtctcgaaaattgat
taatggtattagggataagcaatctggcaaaacaatattagattttttga
aatcagatggttttgccaatcgcaattttatgcagctgatccatgatgat
agtttgacatttaaagaagacattcaaaaagcacaagtgtctggacaagg
cgatagtttacatgaacatattgcaaatttagctggtagccctgctatta
aaaaaggtattttacagactgtaaaagttgttgatgaattggtcaaagta
atggggcggcataagccagaaaatatcgttattgaaatggcacgtgaaaa
tcagacaactcaaaagggccagaaaaattcgcgagagcgtatgaaacgaa
tcgaagaaggtatcaaagaattaggaagtcagattcttaaagagcatcct
gttgaaaatactcaattgcaaaatgaaaagctctatctctattatctcca
aaatggaagagacatgtatgtggaccaagaattagatattaatcgtttaa
gtgattatgatgtcgatcacattgttccacaaagtttccttaaagacgat
tcaatagacaataaggtcttaacgcgttctgataaaaatcgtggtaaatc
ggataacgttccaagtgaagaagtagtcaaaaagatgaaaaactattgga
gacaacttctaaacgccaagttaatcactcaacgtaagtttgataattta
acgaaagctgaacgtggaggtttgagtgaacttgataaagctggttttat
caaacgccaattggttgaaactcgccaaatcactaagcatgtggcacaaa
ttttggatagtcgcatgaatactaaatacgatgaaaatgataaacttatt
cgagaggttaaagtgattaccttaaaatctaaattagtttctgacttccg
aaaagatttccaattctataaagtacgtgagattaacaattaccatcatg
cccatgatgcgtatctaaatgccgtcgttggaactgctttgattaagaaa
tatccaaaacttgaatcggagtttgtctatggtgattataaagtttatga
tgttcgtaaaatgattgctaagtctgagcaagaaataggcaaagcaaccg
caaaatatttcttttactctaatatcatgaacttcttcaaaacagaaatt
acacttgcaaatggagagattcgcaaacgccctctaatcgaaactaatgg
ggaaactggagaaattgtctgggataaagggcgagattttgccacagtgc
gcaaagtattgtccatgccccaagtcaatattgtcaagaaaacagaagta
cagacaggcggattctccaaggagtcaattttaccaaaaagaaattcgga
caagcttattgctcgtaaaaaagactgggatccaaaaaaatatggtggtt
ttgatagtccaacggtagcttattcagtcctagtggttgctaaggtggaa
aaagggaaatcgaagaagttaaaatccgttaaagagttactagggatcac
aattatggaaagaagttcctttgaaaaaaatccgattgactttttagaag
ctaaaggatataaggaagttaaaaaagacttaatcattaaactacctaaa
tatagtctttttgagttagaaaacggtcgtaaacggatgctggctagtgc
cggagaattacaaaaaggaaatgagctggctctgccaagcaaatatgtga
attttttatatttagctagtcattatgaaaagttgaagggtagtccagaa
gataacgaacaaaaacaattgtttgtggagcagcataagcattatttaga
tgagattattgagcaaatcagtgaattttctaagcgtgttattttagcag
atgccaatttagataaagttcttagtgcatataacaaacatagagacaaa
ccaatacgtgaacaagcagaaaatattattcatttatttacgttgacgaa
tcttggagctcccgctgcttttaaatattttgatacaacaattgatcgta
aacgatatacgtctacaaaagaagttttagatgccactcttatccatcaa
tccatcactggtctttatgaaacacgcattgatttgagtcagctaggagg
tgactaa

In some embodiments, the nucleic acid sequence encoding for the defective Cas9 protein consists of the nucleic acid sequence of SEQ ID NO:8.

>dCas9 (addgene #44249)
SEQ ID NO: 8
atggataagaaatactcaataggcttagctatcggcacaaatagcgtcgg
atgggcggtgatcactgatgaatataaggttccgtctaaaaagttcaagg
ttctgggaaatacagaccgccacagtatcaaaaaaaatcttataggggct
cttttatttgacagtggagagacagcggaagcgactcgtctcaaacggac
agctcgtagaaggtatacacgtcggaagaatcgtatttgttatctacagg
agattttttcaaatgagatggcgaaagtagatgatagtttctttcatcga
cttgaagagtcttttttggtggaagaagacaagaagcatgaacgtcatcc
tatttttggaaatatagtagatgaagttgcttatcatgagaaatatccaa
ctatctatcatctgcgaaaaaaattggtagattctactgataaagcggat
ttgcgcttaatctatttggccttagcgcatatgattaagtttcgtggtca
ttttttgattgagggagatttaaatcctgataatagtgatgtggacaaac
tatttatccagttggtacaaacctacaatcaattatttgaagaaaaccct
attaacgcaagtggagtagatgctaaagcgattctttctgcacgattgag
taaatcaagacgattagaaaatctcattgctcagctccccggtgagaaga
aaaatggcttatttgggaatctcattgctttgtcattgggtttgacccct
aattttaaatcaaattttgatttggcagaagatgctaaattacagctttc
aaaagatacttacgatgatgatttagataatttattggcgcaaattggag
atcaatatgctgatttgtttttggcagctaagaatttatcagatgctatt
ttactttcagatatcctaagagtaaatactgaaataactaaggctcccct
atcagcttcaatgattaaacgctacgatgaacatcatcaagacttgactc
ttttaaaagctttagttcgacaacaacttccagaaaagtataaagaaatc
ttttttgatcaatcaaaaaacggatatgcaggttatattgatgggggagc
tagccaagaagaattttataaatttatcaaaccaattttagaaaaaatgg
atggtactgaggaattattggtgaaactaaatcgtgaagatttgctgcgc
aagcaacggacctttgacaacggctctattccccatcaaattcacttggg
tgagctgcatgctattttgagaagacaagaagacttttatccatttttaa
aagacaatcgtgagaagattgaaaaaatcttgacttttcgaattccttat
tatgttggtccattggcgcgtggcaatagtcgttttgcatggatgactcg
gaagtctgaagaaacaattaccccatggaattttgaagaagttgtcgata
aaggtgcttcagctcaatcatttattgaacgcatgacaaactttgataaa
aatcttccaaatgaaaaagtactaccaaaacatagtttgctttatgagta
ttttacggtttataacgaattgacaaaggtcaaatatgttactgaaggaa
tgcgaaaaccagcatttctttcaggtgaacagaagaaagccattgttgat
ttactcttcaaaacaaatcgaaaagtaaccgttaagcaattaaaagaaga
ttatttcaaaaaaatagaatgttttgatagtgttgaaatttcaggagttg
aagatagatttaatgcttcattaggtacctaccatgatttgctaaaaatt
attaaagataaagattttttggataatgaagaaaatgaagatatcttaga
ggatattgttttaacattgaccttatttgaagatagggagatgattgagg
aaagacttaaaacatatgctcacctctttgatgataaggtgatgaaacag
cttaaacgtcgccgttatactggttggggacgtttgtctcgaaaattgat
taatggtattagggataagcaatctggcaaaacaatattagattttttga
aatcagatggttttgccaatcgcaattttatgcagctgatccatgatgat
agtttgacatttaaagaagacattcaaaaagcacaagtgtctggacaagg
cgatagtttacatgaacatattgcaaatttagctggtagccctgctatta
aaaaaggtattttacagactgtaaaagttgttgatgaattggtcaaagta
atggggcggcataagccagaaaatatcgttattgaaatggcacgtgaaaa
tcagacaactcaaaagggccagaaaaattcgcgagagcgtatgaaacgaa
tcgaagaaggtatcaaagaattaggaagtcagattcttaaagagcatcct
gttgaaaatactcaattgcaaaatgaaaagctctatctctattatctcca
aaatggaagagacatgtatgtggaccaagaattagatattaatcgtttaa
gtgattatgatgtcgatgccattgttccacaaagtttccttaaagacgat
tcaatagacaataaggtcttaacgcgttctgataaaaatcgtggtaaatc
ggataacgttccaagtgaagaagtagtcaaaaagatgaaaaactattgga
gacaacttctaaacgccaagttaatcactcaacgtaagtttgataattta
acgaaagctgaacgtggaggtttgagtgaacttgataaagctggttttat
caaacgccaattggttgaaactcgccaaatcactaagcatgtggcacaaa
ttttggatagtcgcatgaatactaaatacgatgaaaatgataaacttatt
cgagaggttaaagtgattaccttaaaatctaaattagtttctgacttccg
aaaagatttccaattctataaagtacgtgagattaacaattaccatcatg
cccatgatgcgtatctaaatgccgtcgttggaactgctttgattaagaaa
tatccaaaacttgaatcggagtttgtctatggtgattataaagtttatga
tgttcgtaaaatgattgctaagtctgagcaagaaataggcaaagcaaccg
caaaatatttcttttactctaatatcatgaacttcttcaaaacagaaatt
acacttgcaaatggagagattcgcaaacgccctctaatcgaaactaatgg
ggaaactggagaaattgtctgggataaagggcgagattttgccacagtgc
gcaaagtattgtccatgccccaagtcaatattgtcaagaaaacagaagta
cagacaggcggattctccaaggagtcaattttaccaaaaagaaattcgga
caagcttattgctcgtaaaaaagactgggatccaaaaaaatatggtggtt
ttgatagtccaacggtagcttattcagtcctagtggttgctaaggtggaa
aaagggaaatcgaagaagttaaaatccgttaaagagttactagggatcac
aattatggaaagaagttcctttgaaaaaaatccgattgactttttagaag
ctaaaggatataaggaagttaaaaaagacttaatcattaaactacctaaa
tatagtctttttgagttagaaaacggtcgtaaacggatgctggctagtgc
cggagaattacaaaaaggaaatgagctggctctgccaagcaaatatgtga
attttttatatttagctagtcattatgaaaagttgaagggtagtccagaa
gataacgaacaaaaacaattgtttgtggagcagcataagcattatttaga
tgagattattgagcaaatcagtgaattttctaagcgtgttattttagcag
atgccaatttagataaagttcttagtgcatataacaaacatagagacaaa
ccaatacgtgaacaagcagaaaatattattcatttatttacgttgacgaa
tcttggagctcccgctgcttttaaatattttgatacaacaattgatcgta
aacgatatacgtctacaaaagaagttttagatgccactcttatccatcaa
tccatcactggtctttatgaaacacgcattgatttgagtcagctaggagg
tgactaa

In some embodiments, the nucleic acid sequence that encodes for the nuclease is operatively linked to a weak constitutive promoter. In some embodiments, the nucleic acid sequence that encodes for the nuclease is operatively linked to the weak constitutive BBa_J23107 promoter having the nucleic acid sequence of SEQ ID NO:9.

> BBa_J23107 promoter
SEQ ID NO: 9
tttacggctagctcagccctaggtattatgctagcAGATCTaaagaggag
aaa

iv) Guide RNA Molecule

In some embodiments, the plasmid of the present invention comprises at 2, 3, 3, 4, 5, 6, 7, 8, 9, or 10 nucleic acid sequences encoding for a guide RNA molecule.

In some embodiments, the guide RNA molecule(s) encoded by the plasmid of the present invention comprises a spacer sequence (i.e. the crRNA) and a trans-activating RNA (“tracrRNA”) sequence.

In some embodiments, the spacer sequence is complementary to any specific target sequence present in a recipient bacterial cell. Typically the spacer sequences are designed by any custom algorithm well known in the art and typically by the custom algorithm CSTB ad described in EXAMPLE.

In some embodiments, the spacer sequence targets a gene that is involved in pathogenicity or other aspects of microbial metabolism. In some embodiments, the spacer sequence is complementary to a target region of a gene that is involved in bacterial metabolism, more particular a gene that is involved in biofilm production.

In some embodiments, the spacer sequence targets an antibiotic resistance gene. For example, there are many resistance genes encoding beta-lactamases (bla genes) giving resistance to a large range of different beta-lactam antibiotics. In some embodiments, the plasmid of the present invention transcribes one or more RNA guide molecules each comprising a spacer sequence sufficiently complementary to a target sequence of one or more beta-lactamase genes. For example, the one or more RNA guide molecules may target one or more or all of the genes selected from the group consisting of: NDM, VIM, IMP, KPC, OXA, TEM, SHV, CTX, OKP, LEN, GES, MIR, ACT, ACC, CMY, LAT, and FOX. In particular, the one or more RNA guide molecules may comprise a spacer sequence sufficiently complementary to target sequences of the beta lactam family of antibiotic resistance genes, including one or more or all of the following: a first spacer sequence sufficiently complementary to target sequences for NDM-1, -2, -10; a second spacer sufficiently complementary to target sequences for VIM-1, -2, -4, -12, -19, -26, -27-33, 34; a third spacer sufficiently complementary to target sequences for IMP-32, -38, -48; a fourth spacer sufficiently complementary to target sequences for KPC-1, -2, -3, -4, -6, -7, -8, -11, -12, -14, -15, -16, -17; a fifth spacer sufficiently complementary to target sequences for OXA-48; a sixth spacer sufficiently complementary to target sequences for TEM-1, -1, -3, -139, -162, -183, -192, -197, -198, -209, a seventh spacer sufficiently complementary to target sequences for SHV and its variants; and an eighth spacer sufficiently complementary to target sequences for CTX and its variants.

In some embodiments, the spacer sequence is designed to generate a DSB in the target sequence. For instance, where the target sequence is located on a chromosome or a replicon (e.g. a plasmid) such as a bacterial chromosome or plasmid, then a DSB can lead to degradation and hence loss of the chromosome or replicon. If the target sequence is located on a bacterial chromosome then the recipient may die directly as a consequence of the DSB. Additionally, some natural plasmids carry killing functions that only become toxic if the cell loses the plasmid, which is a natural mechanism to ensure faithful inheritance of plasmids in dividing cells. If a plasmid carrying the target sequence of the antibiotic resistance gene also carries such a killing function, and the plasmid is lost as a result of the DSB generated, the cell may die.

In some embodiments, the spacer sequence is encoded by a nucleic acid sequence selected from the Table A.

| | | Target |
|---|---|---|
| Primer | Sequence | sequence |
| OL596 | TAGTCCATTACGGTCAATCCGCCGT | lacZ2 |
| | TTGTTCCCAG (SEQ ID NO: | (Cui and |
| | 10) | Bikard, |
| | | 2016) |
| OL597 | AAACTGGGAACAAACGGCGGATTGA | |
| | CCGTAATGGA (SEQ ID NO: | |
| | 11) | |
| OL715 | TAGTCACCAAAAACACAGCCGATAG | OXA48 |
| | (SEQ ID NO: 12) | |
| OL716 | AAACTATCGGCTGTGTTTTTGGTGA | |
| | (SEQ ID NO: 13) | |
| OL668 | TAGTTGTTTTTCATGTTGTCACCCG | csgB |
| | (SEQ ID NO: 14) | |
| OL669 | AAACGGGTGACAACATGAAAAACAA | |
| | (SEQ ID NO: 15) | |
| OL612 | TAGTTGTAATCACAATGATGAAAAG | CR1 |
| | (SEQ ID NO: 16) | |
| OL613 | AAACTTTTCATCATTGTGATTACAA | |
| | (SEQ ID NO: 17) | |
| OL635 | TAGTAGCGCCAACCGATATCACCAG | CR22 |
| | (SEQ ID NO: 18) | |
| OL636 | AAACTGGTGATATCGGTTGGCGCTA | |
| | (SEQ ID NO: 19) | |
| OL725 | TAGTTGCCATCACTACTGTGCG | Ecl |
| | (SEQ ID NO: 20) | |
| OL726 | AAACGCACAGTAGTGATGGCAA | |
| | (SEQ ID NO: 21 | |
| OL810 | TAGTTGGCGTTGAAGACGACGG | EPEC |
| | (SEQ ID NO: 22) | |
| OL811 | AAACCGTCGTCTTCAACGCCAA | |
| | (SEQ ID NO: 23) | |
| OL828 | TAGTGATATCAAAGTGGTTATG | EEC |
| | (SEQ ID NO: 24) | |

In some embodiments, the nucleic acid sequence encoding for the space sequence is flanked at each end by restriction sites so as to allow the quick cloning of said spacer sequence in the plasmid of the present invention.

In some embodiments, the tracrRNA sequence of the guide RNA molecule(s) is encoded by the nucleic acid sequence of SEQ ID NO:25.

>tracrRNA sequence
SEQ ID NO: 25
gttttagagctagaaatagcaagttaaaataaggctagtccgttatcaac
ttgaaaaagtggcaccgagtcggtgc

In some embodiments, the nucleic acid sequence(s) that encodes for guide RNA molecule is (are) operatively linked to a strong constitutive promoter. In some embodiments, the nucleic acid sequence(s) that encode(s) for the guide RNA molecule is operatively linked to the strong constitutive BBa_J23119 promoter having the nucleic acid sequence of SEQ ID NO:26.

| | >BBa J23119 promoter |
|---|---|
| SEQ ID NO: 26 | |
| | ttgacagctagctcagtcctaggtataatactagt |

v) Optional Selection Marker Genes

In some embodiments, the plasmid of the present invention optionally comprises one or more selection marker. Transformed cells containing the plasmid of the present invention may indeed be selected with a variety of positive and/or negative selection markers.

For instance, a positive selection marker can be a gene that allows growth in the absence of an essential nutrient, such as an amino acid. A variety of suitable positive/negative selection pairs are available in the art. For example, various amino acid analogs known in the art could be used as a negative selection, while growth on minimal media (relative to the amino acid analog) could be used as a positive selection.

Visually detectable markers are also suitable for being uses in the present invention, and may be positively and negatively selected and/or screened using technologies such as fluorescence activated cell sorting (FACS) or microfluidics. Examples of detectable markers include various enzymes, prosthetic groups, fluorescent markers, luminescent markers, bioluminescent markers, and the like. Examples of suitable fluorescent proteins include, but are not limited to, yellow fluorescent protein (YFP), green fluorescence protein (GFP), cyan fluorescence protein (CFP), umbelliferone, fluorescein, fluorescein isothiocyanate, rhodamine, dichiorotriazinylamine fluorescein, dansyl chloride, phycoerythrin and the like. Examples of suitable bioluminescent markers include, but are not limited to, luciferase (e.g., bacterial, firefly, click beetle and the like), luciferin, aequorin and the like. Examples of suitable enzyme systems having visually detectable signals include, but are not limited to, galactosidases, glucorinidases, phosphatases, peroxidases, cholinesterases and the like.

In some embodiments, the positive selection marker is a gene that confers resistance to a compound, which would be lethal to the cell in the absence of the gene. For example, a cell expressing an antibiotic resistance gene would survive in the presence of an antibiotic, while a cell lacking the gene would not. Suitable antibiotic resistance genes include, but are not limited to, genes such as ampicillin-resistance gene, neomycin-resistance gene, blasticidin-resistance gene, hygromycin-resistance gene, puromycin-resistance gene, chloramphenicol-resistance gene, apramycin-resistance gene and the like.

vi) Specific Plasmids:

In some embodiments, the plasmid of the present invention consists of the nucleic acid sequence as set forth in SEQ ID NO:27 or 28.

>TAP_{Kn}-Cas9-nsp
SEQ ID NO: 27
atctttgacagctagctcagtcctaggtataatactagtgaagagcACCGGTgctcttcgttttagagc
tagaaatagcaagttaaaataaggctagtccgttatcaacttgaaaaagtggcaccgagtcggtgcttt
ttttgaagcttgggcccgaacaaaaaaaaaccccgcccctgacagggcggggttttttttgtttgtcgg
tgaactggatccttaccagctgggcgcgccccccctacgggcttgctctccgggcttcgccctgcgcgg
tcgctgcgctcccttgccagcccgtggatatgtggacgatggccgcgagcggccaccggctggctcgct
tcgctcggcccgtggacaaccctgctggacaagctgatggacaggctgcgcctgcccacgagcttgacc
acagggattgcccaccggctacccagccttcgaccacatacccaccggctccaactgcgcggcctgcgg
ccttgccccatcaatttttttaattttctctggggaaaagcctccggcctgcggcctgcgcgcttcgct
tgccggttggacaccaagtggaaggcgggtcaaggctcgcgcagcgaccgcgcagcggcttggccttga
cgcgcctggaacgacccaagcctatgcgagtgggggcagtcgaagggcgaagcccgcccgcctgccccc
cgagcctcacggcggcgagtgcgggggttccaagggggcagcgccaccttgggcaaggccgaaggccgc
gcagtcgatcaacaagccccggaggggccactttttgccggagggggagccgcgccgaaggcgtggggg
aaccccgcaggggtgcccttctttgggcaccaaagaactagatatagggcgaaatgcgaaagacttaaa
aatcaacaacttaaaaaaggggggtacgcaacagctcattgcggcaccccccgcaatagctcattgcgt
aggttaaagaaaatctgtaattgactgccacttttacgcaacgcataattgttgtcgcgctgccgaaaa
gttgcagctgattgcgcatggtgccgcaaccgtgcggcacccctaccgcatggagataagcatggccac
gcagtccagagaaatcggcattcaagccaagaacaagcccggtcactgggtgcaaacggaacgcaaagc
gcatgaggcgtgggccgggcttattgcgaggaaacccacggcggcaatgctgctgcatcacctcgtggc
gcagatgggccaccagaacgccgtggtggtcagccagaagacactttccaagctcatcggacgttcttt
gcggacggtccaatacgcagtcaaggacttggtggccgagcgctggatctccgtcgtgaagctcaacgg
ccccggcaccgtgtcggcctacgtggtcaatgaccgcgtggcgtggggccagccccgcgaccagttgcg
cctgtcggtgttcagtgccgccgtggtggttgatcacgacgaccaggacgaatcgctgttggggcatgg
cgacctgcgccgcatcccgaccctgtatccgggcgagcagcaactaccgaccggccccggcgaggagcc
gcccagccagcccggcattccgggcatggaaccagacctgccagccttgaccgaaacggaggaatggga
acggcgcgggcagcagcgcctgccgatgcccgatgagccgtgttttctggacgatggcgagccgttgga
gccgccgacacgggtaacgctgccgcgccggtagggcatgcaaacagggacgcaccgctagcagcgccc
ctagcggtatcctataaaaaaacacaccgcgccgctagcagcacccctaatataaaataatgtttttta
taaaaatagtcagtaccacccctacaaaacggtgtcggcgcgttgttgtagccgcgccgacaccgcttt
tttaaatatcataaagagagtaagagaaactaatttttcataacactctatttataaagaaaaatcagc
aaaaacttgtttttgcgtggggtgtggtgcttttggtggtgagaaccaccaacctgttgagcctttttg
tggagcgcgcggaccgcggtccagagagcgttcaccgacaaacaacagataaaacgaaaggcccagtct
ttcgactgagcctttcgttttatttgatgcctggagatccttactcgagttagtcacctcctagctgac
tcaaatcaatgcgtgtttcataaagaccagtgatggattgatggataagagtggcatctaaaacttctt
ttgtagacgtatatcgtttacgatcaattgttgtatcaaaatatttaaaagcagcgggagctccaagat
tcgtcaacgtaaataaatgaataatattttctgcttgttcacgtattggtttgtctctatgtttgttat
atgcactaagaactttatctaaattggcatctgctaaaataacacgcttagaaaattcactgatttgct
caataatctcatctaaataatgcttatgctgctccacaaacaattgtttttgttcgttatcttctggac
tacccttcaacttttcataatgactagctaaatataaaaaattcacatatttgcttggcagagccagct
catttcctttttgtaattctccggcactagccagcatccgtttacgaccgttttctaactcaaaaagac
tatatttaggtagtttaatgattaagtcttttttaacttccttatatcctttagcttctaaaaagtcaa
tcggatttttttcaaaggaacttctttccataattgtgatccctagtaactctttaacggattttaact
tcttcgatttccctttttccaccttagcaaccactaggactgaataagctaccgttggactatcaaaac
caccatatttttttggatcccagtcttttttacgagcaataagcttgtccgaatttctttttggtaaaa
ttgactccttggagaatccgcctgtctgtacttctgttttcttgacaatattgacttggggcatggaca
atactttgcgcactgtggcaaaatctcgccctttatcccagacaatttctccagtttccccattagttt
cgattagagggcgtttgcgaatctctccatttgcaagtgtaatttctgttttgaagaagttcatgatat
tagagtaaaagaaatattttgcggttgctttgcctatttcttgctcagacttagcaatcattttacgaa
catcataaactttataatcaccatagacaaactccgattcaagttttggatatttcttaatcaaagcag
ttccaacgacggcatttagatacgcatcatgggcatgatggtaattgttaatctcacgtactttataga
attggaaatcttttcggaagtcagaaactaatttagattttaaggtaatcactttaacctctcgaataa
gtttatcattttcatcgtatttagtattcatgcgactatccaaaatttgtgccacatgcttagtgattt
ggcgagtttcaaccaattggcgtttgataaaaccagctttatcaagttcactcaaacctccacgttcag
ctttcgttaaattatcaaacttacgttgagtgattaacttggcgtttagaagttgtctccaatagtttt
tcatctttttgactacttcttcacttggaacgttatccgatttaccacgatttttatcagaacgcgtta
agaccttattgtctattgaatcgtctttaaggaaactttgtggaacaatgtgatcgacatcataatcac
ttaaacgattaatatctaattcttggtccacatacatgtctcttccattttggagataatagagataga
gcttttcattttgcaattgagtattttcaacaggatgctctttaagaatctgacttcctaattctttga
taccttcttcgattcgtttcatacgctctcgcgaatttttctggcccttttgagttgtctgattttcac
gtgccatttcaataacgatattttctggcttatgccgccccattactttgaccaattcatcaacaactt
ttacagtctgtaaaataccttttttaatagcagggctaccagctaaatttgcaatatgttcatgtaaac
tatcgccttgtccagacacttgtgctttttgaatgtcttctttaaatgtcaaactatcatcatggatca
gctgcataaaattgcgattggcaaaaccatctgatttcaaaaaatctaatattgttttgccagattgct
tatccctaataccattaatcaattttcgagacaaacgtccccaaccagtataacggcgacgtttaagct
gtttcatcaccttatcatcaaagaggtgagcatatgttttaagtctttcctcaatcatctccctatctt
caaataaggtcaatgttaaaacaatatcctctaagatatcttcattttcttcattatccaaaaaatctt
tatctttaataatttttagcaaatcatggtaggtacctaatgaagcattaaatctatcttcaactcctg
aaatttcaacactatcaaaacattctatttttttgaaataatcttcttttaattgcttaacggttactt
ttcgatttgttttgaagagtaaatcaacaatggctttcttctgttcacctgaaagaaatgctggttttc
gcattccttcagtaacatatttgacctttgtcaattcgttataaaccgtaaaatactcataaagcaaac
tatgttttggtagtactttttcatttggaagatttttatcaaagtttgtcatgcgttcaataaatgatt
gagctgaagcacctttatcgacaacttcttcaaaattccatggggtaattgtttcttcagacttccgag
tcatccatgcaaaacgactattgccacgcgccaatggaccaacataataaggaattcgaaaagtcaaga
ttttttcaatcttctcacgattgtcttttaaaaatggataaaagtcttcttgtcttctcaaaatagcat
gcagctcacccaagtgaatttgatggggaatagagccgttgtcaaaggtccgttgcttgcgcagcaaat
cttcacgatttagtttcaccaataattcctcagtaccatccattttttctaaaattggtttgataaatt
tataaaattcttcttggctagctcccccatcaatataacctgcatatccgttttttgattgatcaaaaa
agatttctttatacttttctggaagttgttgtcgaactaaagcttttaaaagagtcaagtcttgatgat
gttcatcgtagcgtttaatcattgaagctgataggggagccttagttatttcagtatttactcttagga
tatctgaaagtaaaatagcatctgataaattcttagctgccaaaaacaaatcagcatattgatctccaa
tttgcgccaataaattatctaaatcatcatcgtaagtatcttttgaaagctgtaatttagcatcttctg
ccaaatcaaaatttgatttaaaattaggggtcaaacccaatgacaaagcaatgagattcccaaataagc
catttttcttctcaccggggagctgagcaatgagattttctaatcgtcttgatttactcaatcgtgcag
aaagaatcgctttagcatctactccacttgcgttaatagggttttcttcaaataattgattgtaggttt
gtaccaactggataaatagtttgtccacatcactattatcaggatttaaatctccctcaatcaaaaaat
gaccacgaaacttaatcatatgcgctaaggccaaatagattaagcgcaaatccgctttatcagtagaat
ctaccaatttttttcgcagatgatagatagttggatatttctcatgataagcaacttcatctactatat
ttccaaaaataggatgacgttcatgcttcttgtcttcttccaccaaaaaagactcttcaagtcgatgaa
agaaactatcatctactttcgccatctcatttgaaaaaatctcctgtagataacaaatacgattcttcc
gacgtgtataccttctacgagctgtccgtttgagacgagtcgcttccgctgtctctccactgtcaaata
aaagagcccctataagattttttttgatactgtggcggtctgtatttcccagaaccttgaactttttag
acggaaccttatattcatcagtgatcaccgcccatccgacgctatttgtgccgatatctaagcctattg
agtatttcttatccatagatCCTTTCTCCTCTTTAGATCTgctagcataatacctagggctgagctagc
cgtaaaTGAGGTTTCAGCAAAAAACCcctcaagacccgtttagaggccccaaggggttatgctagttat
tgctcagcggattaattattagaaaaattcatccagcatcagatgaaattgcagtttgttcatatccgg
attatcaatgccatatttctgaaacagacgtttttgcaggctcgggctaaattcgcccaggcagttcca
cagaatggccagatcctgataacgatccgcaatgcccacacggcccacatcaatgcagccaatcagttt
gccttcatcgaaaatcaggttatccaggctaaaatcgccgtgggtcaccacgctatccgggctaaacgg
cagcagtttatgcatttctttccacacctgttccaccggccagccgttacgttcatcatcaaaatcgct
cgcatccaccaggccgttgttcatacggctctgcgcctgggccagacgaaacacacgatcgctgttaaa
cgggcagttgcacaccggaatgctatgcagacgacgcagaaacacggccagcgcatccacaatgttttc
gccgctatccggatattcttccagcacctgaaacgcggttttgcccggaatcgcggtggtcagcagcca
cgcatcatccggggtgcgaataaaatgtttaatggtcggcagcggcataaattcggtcagccagttcag
acgcaccatttcatcggtcacatcgttcgccacgctgcctttgccatgtttcagaaacagttccggcgc
atccggtttgccatacagacgataaatggtcgcgccgctctgacccacgttatcacgcgcccatttata
gccatacagatccgcatccatgttgctgttcagacgcggacggctacagctcgtttcacgctgaatatg
gctcataacaccccttgtattactgtttatgtaagcagacagttttattgttcatgatgatatattttt
atcttgtgcaatgtaacatcagagattttgagacacaaatttaaatcgtaattattggggacccctgga
ttctcaccaataaaaaacgcccggcggcaaccgagcgttctgaacaaatccagatggagttctgaggtc
attactggatctatcaacaggagtccaagactagtcgccagggttttcccagtcacgacgcggccgcaa
gcttgcatgcctgcaggtcgactctagaggatccccgggtaccgagctcgaattcgcgcggccgcggcc
taggcggcctcctgtgtgaaattgttatccgctttaattaa
>TAP_{Kn}-dCas9-nsp
SEQ ID NO: 28
atctttgacagctagctcagtcctaggtataatactagtgaagagcACCGGTgctcttcgttttagagc
tagaaatagcaagttaaaataaggctagtccgttatcaacttgaaaaagtggcaccgagtcggtgcttt
ttttgaagcttgggcccgaacaaaaaaaaaccccgcccctgacagggcggggttttttttgtttgtcgg
tgaactggatccttaccagctgggcgcgccccccctacgggcttgctctccgggcttcgccctgcgcgg
tcgctgcgctcccttgccagcccgtggatatgtggacgatggccgcgagcggccaccggctggctcgct
tcgctcggcccgtggacaaccctgctggacaagctgatggacaggctgcgcctgcccacgagcttgacc
acagggattgcccaccggctacccagccttcgaccacatacccaccggctccaactgcgcggcctgcgg
ccttgccccatcaatttttttaattttctctggggaaaagcctccggcctgcggcctgcgcgcttcgct
tgccggttggacaccaagtggaaggcgggtcaaggctcgcgcagcgaccgcgcagcggcttggccttga
cgcgcctggaacgacccaagcctatgcgagtgggggcagtcgaagggcgaagcccgcccgcctgccccc
cgagcctcacggcggcgagtgcgggggttccaagggggcagcgccaccttgggcaaggccgaaggccgc
gcagtcgatcaacaagccccggaggggccactttttgccggagggggagccgcgccgaaggcgtggggg
aaccccgcaggggtgcccttctttgggcaccaaagaactagatatagggcgaaatgcgaaagacttaaa
aatcaacaacttaaaaaaggggggtacgcaacagctcattgcggcaccccccgcaatagctcattgcgt
aggttaaagaaaatctgtaattgactgccacttttacgcaacgcataattgttgtcgcgctgccgaaaa
gttgcagctgattgcgcatggtgccgcaaccgtgcggcacccctaccgcatggagataagcatggccac
gcagtccagagaaatcggcattcaagccaagaacaagcccggtcactgggtgcaaacggaacgcaaagc
gcatgaggcgtgggccgggcttattgcgaggaaacccacggcggcaatgctgctgcatcacctcgtggc
gcagatgggccaccagaacgccgtggtggtcagccagaagacactttccaagctcatcggacgttcttt
gcggacggtccaatacgcagtcaaggacttggtggccgagcgctggatctccgtcgtgaagctcaacgg
ccccggcaccgtgtcggcctacgtggtcaatgaccgcgtggcgtggggccagccccgcgaccagttgcg
cctgtcggtgttcagtgccgccgtggtggttgatcacgacgaccaggacgaatcgctgttggggcatgg
cgacctgcgccgcatcccgaccctgtatccgggcgagcagcaactaccgaccggccccggcgaggagcc
gcccagccagcccggcattccgggcatggaaccagacctgccagccttgaccgaaacggaggaatggga
acggcgcgggcagcagcgcctgccgatgcccgatgagccgtgttttctggacgatggcgagccgttgga
gccgccgacacgggtaacgctgccgcgccggtagggcatgcaaacagggacgcaccgctagcagcgccc
ctagcggtatcctataaaaaaacacaccgcgccgctagcagcacccctaatataaaataatgtttttta
taaaaatagtcagtaccacccctacaaaacggtgtcggcgcgttgttgtagccgcgccgacaccgcttt
tttaaatatcataaagagagtaagagaaactaatttttcataacactctatttataaagaaaaatcagc
aaaaacttgtttttgcgtggggtgtggtgcttttggtggtgagaaccaccaacctgttgagcctttttg
tggagcgcgcggaccgcggtccagagagcgttcaccgacaaacaacagataaaacgaaaggcccagtct
ttcgactgagcctttcgttttatttgatgcctggagatccttactcgagttagtcacctcctagctgac
tcaaatcaatgcgtgtttcataaagaccagtgatggattgatggataagagtggcatctaaaacttctt
ttgtagacgtatatcgtttacgatcaattgttgtatcaaaatatttaaaagcagcgggagctccaagat
tcgtcaacgtaaataaatgaataatattttctgcttgttcacgtattggtttgtctctatgtttgttat
atgcactaagaactttatctaaattggcatctgctaaaataacacgcttagaaaattcactgatttgct
caataatctcatctaaataatgcttatgctgctccacaaacaattgtttttgttcgttatcttctggac
tacccttcaacttttcataatgactagctaaatataaaaaattcacatatttgcttggcagagccagct
catttcctttttgtaattctccggcactagccagcatccgtttacgaccgttttctaactcaaaaagac
tatatttaggtagtttaatgattaagtcttttttaacttccttatatcctttagcttctaaaaagtcaa
tcggatttttttcaaaggaacttctttccataattgtgatccctagtaactctttaacggattttaact
tcttcgatttccctttttccaccttagcaaccactaggactgaataagctaccgttggactatcaaaac
caccatatttttttggatcccagtcttttttacgagcaataagcttgtccgaatttctttttggtaaaa
ttgactccttggagaatccgcctgtctgtacttctgttttcttgacaatattgacttggggcatggaca
atactttgcgcactgtggcaaaatctcgccctttatcccagacaatttctccagtttccccattagttt
cgattagagggcgtttgcgaatctctccatttgcaagtgtaatttctgttttgaagaagttcatgatat
tagagtaaaagaaatattttgcggttgctttgcctatttcttgctcagacttagcaatcattttacgaa
catcataaactttataatcaccatagacaaactccgattcaagttttggatatttcttaatcaaagcag
ttccaacgacggcatttagatacgcatcatgggcatgatggtaattgttaatctcacgtactttataga
attggaaatcttttcggaagtcagaaactaatttagattttaaggtaatcactttaacctctcgaataa
gtttatcattttcatcgtatttagtattcatgcgactatccaaaatttgtgccacatgettagtgattt
ggcgagtttcaaccaattggcgtttgataaaaccagctttatcaagttcactcaaacctccacgttcag
ctttcgttaaattatcaaacttacgttgagtgattaacttggcgtttagaagttgtctccaatagtttt
tcatctttttgactacttcttcacttggaacgttatccgatttaccacgatttttatcagaacgcgtta
agaccttattgtctattgaatcgtctttaaggaaactttgtggaacaatggcatcgacatcataatcac
ttaaacgattaatatctaattcttggtccacatacatgtctcttccattttggagataatagagataga
gcttttcattttgcaattgagtattttcaacaggatgctctttaagaatctgacttcctaattctttga
taccttcttcgattcgtttcatacgctctcgcgaatttttctggcccttttgagttgtctgattttcac
gtgccatttcaataacgatattttctggcttatgccgccccattactttgaccaattcatcaacaactt
ttacagtctgtaaaataccttttttaatagcagggctaccagctaaatttgcaatatgttcatgtaaac
tatcgccttgtccagacacttgtgctttttgaatgtcttctttaaatgtcaaactatcatcatggatca
gctgcataaaattgcgattggcaaaaccatctgatttcaaaaaatctaatattgttttgccagattgct
tatccctaataccattaatcaattttcgagacaaacgtccccaaccagtataacggcgacgtttaagct
gtttcatcaccttatcatcaaagaggtgagcatatgttttaagtctttcctcaatcatctccctatctt
caaataaggtcaatgttaaaacaatatcctctaagatatcttcattttcttcattatccaaaaaatctt
tatctttaataatttttagcaaatcatggtaggtacctaatgaagcattaaatctatcttcaactcctg
aaatttcaacactatcaaaacattctatttttttgaaataatcttcttttaattgcttaacggttactt
ttcgatttgttttgaagagtaaatcaacaatggctttcttctgttcacctgaaagaaatgctggttttc
gcattccttcagtaacatatttgacctttgtcaattcgttataaaccgtaaaatactcataaagcaaac
tatgttttggtagtactttttcatttggaagatttttatcaaagtttgtcatgcgttcaataaatgatt
gagctgaagcacctttatcgacaacttcttcaaaattccatggggtaattgtttcttcagacttccgag
tcatccatgcaaaacgactattgccacgcgccaatggaccaacataataaggaattcgaaaagtcaaga
ttttttcaatcttctcacgattgtcttttaaaaatggataaaagtcttcttgtcttctcaaaatagcat
gcagctcacccaagtgaatttgatggggaatagagccgttgtcaaaggtccgttgcttgcgcagcaaat
cttcacgatttagtttcaccaataattcctcagtaccatccattttttctaaaattggtttgataaatt
tataaaattcttcttggctagctcccccatcaatataacctgcatatccgttttttgattgatcaaaaa
agatttctttatacttttctggaagttgttgtcgaactaaagcttttaaaagagtcaagtcttgatgat
gttcatcgtagcgtttaatcattgaagctgataggggagccttagttatttcagtatttactcttagga
tatctgaaagtaaaatagcatctgataaattcttagctgccaaaaacaaatcagcatattgatctccaa
tttgcgccaataaattatctaaatcatcatcgtaagtatcttttgaaagctgtaatttagcatcttctg
ccaaatcaaaatttgatttaaaattaggggtcaaacccaatgacaaagcaatgagattcccaaataagc
catttttcttctcaccggggagctgagcaatgagattttctaatcgtcttgatttactcaatcgtgcag
aaagaatcgctttagcatctactccacttgcgttaatagggttttcttcaaataattgattgtaggttt
gtaccaactggataaatagtttgtccacatcactattatcaggatttaaatctccctcaatcaaaaaat
gaccacgaaacttaatcatatgcgctaaggccaaatagattaagcgcaaatccgctttatcagtagaat
ctaccaatttttttcgcagatgatagatagttggatatttctcatgataagcaacttcatctactatat
ttccaaaaataggatgacgttcatgcttcttgtcttcttccaccaaaaaagactcttcaagtcgatgaa
agaaactatcatctactttcgccatctcatttgaaaaaatctcctgtagataacaaatacgattcttcc
gacgtgtataccttctacgagctgtccgtttgagacgagtcgcttccgctgtctctccactgtcaaata
aaagagcccctataagattttttttgatactgtggcggtctgtatttcccagaaccttgaactttttag
acggaaccttatattcatcagtgatcaccgcccatccgacgctatttgtgccgatagctaagcctattg
agtatttcttatccatagatCCTTTCTCCTCTTTAGATCTgctagcataatacctagggctgagctagc
cgtaaaTGAGGTTTCAGCAAAAAACCcctcaagacccgtttagaggccccaaggggttatgctagttat
tgctcagcggattaattattagaaaaattcatccagcatcagatgaaattgcagtttgttcatatccgg
attatcaatgccatatttctgaaacagacgtttttgcaggctcgggctaaattcgcccaggcagttcca
cagaatggccagatcctgataacgatccgcaatgcccacacggcccacatcaatgcagccaatcagttt
gccttcatcgaaaatcaggttatccaggctaaaatcgccgtgggtcaccacgctatccgggctaaacgg
cagcagtttatgcatttctttccacacctgttccaccggccagccgttacgttcatcatcaaaatcgct
cgcatccaccaggccgttgttcatacggctctgcgcctgggccagacgaaacacacgatcgctgttaaa
cgggcagttgcacaccggaatgctatgcagacgacgcagaaacacggccagcgcatccacaatgttttc
gccgctatccggatattcttccagcacctgaaacgcggttttgcccggaatcgcggtggtcagcagcca
cgcatcatccggggtgcgaataaaatgtttaatggtcggcagcggcataaattcggtcagccagttcag
acgcaccatttcatcggtcacatcgttcgccacgctgcctttgccatgtttcagaaacagttccggcgc
atccggtttgccatacagacgataaatggtcgcgccgctctgacccacgttatcacgcgcccatttata
gccatacagatccgcatccatgttgctgttcagacgcggacggctacagctcgtttcacgctgaatatg
gctcataacaccccttgtattactgtttatgtaagcagacagttttattgttcatgatgatatattttt
atcttgtgcaatgtaacatcagagattttgagacacaaatttaaatcgtaattattggggacccctgga
ttctcaccaataaaaaacgcccggcggcaaccgagcgttctgaacaaatccagatggagttctgaggtc
attactggatctatcaacaggagtccaagactagtcgccagggttttcccagtcacgacgcggccgcaa
gcttgcatgcctgcaggtcgactctagaggatccccgggtaccgagctcgaattcgcgcggccgcggcc
taggcggcctcctgtgtgaaattgttatccgctttaattaa

vii) Methods of Producing Plasmids

The plasmid of the present invention may be produced using conventional molecular biology and recombinant DNA techniques within the skill of the art. Such techniques are explained fully in the literature. See, e.g., Sambrook, Fritsch & Maniatis, Molecular Cloning: A Laboratory Manual, Second Edition (1989) Cold Spring Harbor Laboratory Press, Cold Spring Harbor, New York (herein “Sambrook et al., 1989”); DNA Cloning: A Practical Approach, Volumes I and II (D. N. Glover ed. 1985); F. M. Ausubel et al. (eds.), Current Protocols in Molecular Biology, John Wiley & Sons, Inc. (1994).

Donor Bacterial Cells:

A further object of the present invention is a donor bacterial cell comprising a copy number of the targeted-antibacterial-plasmid of the present invention.

The donor bacterial cell of the present also comprises conjugative transfer genes and thus typical comprises a copy number of conjugative plasmids. Conjugative transfer (tra) genes have been characterized in many conjugative bacterial plasmids. The interchangeability between the gene modules conferring the ranges of hosts susceptible for conjugal transfer include Gram-positive and Gram-negative species. Examples of characterized tra genes that are suitable for use in the present invention are the tra genes from: (1) F (Firth, N., Ippen-Ihler, K. and Skurray, R. A. 1996, Structure and function of F factor and mechanism of conjugation. In: Escherichia coli and Salmonella, Neidhard et al., eds., ASM Press, Washington D.C.); (2) RP4 (Nunez, B., Avila, P. and de la Cruz, 1997, Genes involved in conjugative DNA processing. Mol. Microbial. 24: 1157-1168); and (3) Ti (Ferrand, S. K., Hwang, I. and Cook, D. M. 1996, The tra region of Nopaline type Ti plasmid is a chimera with elements related to the transfer systems of RSF1010, RP4 and F. J. Bacteriol. 178: 4233-4247).

In some embodiments, the donor bacterial cell of the present invention comprises a copy number of F factors and a copy number of targeted-antibacterial-plasmids that comprises the origin transfer of plasmid F.

In some embodiments, the donor bacterial cell of the present invention comprises a copy number of RP4 plasmids and a copy number of targeted-antibacterial-plasmids that comprises the origin transfer of plasmid RP4.

In some embodiments, the donor bacterial cell is selected from the group consisting of the family Acidobacteriaceae, Actinomycetaceae, Actinopolysporaceae, Corynebacteriaceae, Gordoniaceae, Mycobacteriaceae, Nocardiaceae, Brevibacteriaceae, Cellulomonadaceae, Demequinaceae, Microbacteriaceae, Micrococcaceae, Promicromonosporaceae, Aarobacteraceae, Micromonosporaceae, Nocardioidaceae, Propionibacteriaceae, Actinosynnemataceae, Pseudonocardiaceae, Streptomycetaceae, Nocardiopsaceae, Streptosporangiaceae, Thermomonosporaceae, Bifidobacteriaceae, Bacteroidaceae, Marinilabiliaceae, Morphyromonadaceae, Cyclobacteriaceae, Cytophagaceae, Flammeovirgaceae, Rhodothermaceae, Blattabacteriaceae, Cryomorphaceae, Flavobacteriaceae, Schleiferiaceae, Chitinophagaceae, Saprospiraceae, Sphingobacteriaceae, Chlamydiaceae, Parachlamydiaceae, Simkaniaceae, Waddliaceae, Bacillaceae, Listeriaceae, Paenibacillaceae, Planococcaceae, Staphylococcaceae, Aerococcaceae, Carnobacteriaceae, Enterococcaceae, Lactobacillaceae, Leuconostocaceae, Streptococcaceae, Clostridiaceae, Eubacteriaceae, Heliobacteriaceae, Peptococcaceae, Ruminococcaceae, Caulobacteraceae, Hyphomonadaceae, Bradyrhizobiaceae, Brucellaceae, Hyphomicrobiaceae, Methylobacteriaceae, Rhizobiaceae, Xanthobacteraceae, Rhodobacteraceae, Acetobacteraceae, Rhodospirillaceae, Alcaligenaceae, Burkholderiaceae, Comamonadaceae, Oxalobacteraceae, Neisseriaceae, Spirillaceae, Rhodocyclaceae, Desulfobacteraceae, Cystobacteraceae, Myxococcaceae, Nannocystaceae, Phaselicystidaceae, Polyangiaceae, Sandaracinaceae, Syntrophaceae, Syntrophobacteraceae, Syntrophorhabdaceae, Campylobacteraceae, Helicobacteraceae, Acidithiobacillaceae, Aeromonadaceae, Succinivibrionaceae, Alteromonadaceae, Celerinatantimonadaceae, Colwelliaceae, Ferrimonadaceae, Idiomarinaceae, Moritellaceae, Pseudoalteromonadaceae, Psychromonadaceae, Shewanellaceae, Chromatiaceae, Ectothiorhodospiraceae, Granulosicoccaceae, Enterobacteriaceae, Coxiellaceae, Legionellaceae, Alcanivoracaceae, Hahellaceae, Halomonadaceae, Litoricolaceae, Pasteurellaceae, Moraxellaceae, Pseudomonadaceae, Vibrionaceae, Nevskiaceae, Sinobacteraceae, thomonadaceae Brachyspiraceae, Brevinemataceae, Leptospiraceae, Spirochaetaceae, Haloplasmataceae and Mycoplasmataceae.

In some embodiments, the donor bacterial cell is selected from the group consisting of Streptomyces ambofaciens, Streptomyces avermitilis, Streptomyces capreolus, Streptomyces carcinostaticus, Streptomyces cervinus, Streptomyces clavuligerus, Streptomyces davawensis, Streptomyces fradiae, Streptomyces griseus, Streptomyces hygroscopicus, Streptomyces lavendulae, Streptomyces lividans, Streptomyces natalensis, Streptomyces noursei, Streptomyces kanamyceticus, Streptomyces nodosum, Streptomyces tsukubaensis, Streptomyces sp., Streptomyces coelicolor, Streptomyces cinnamonensis, Streptomyces platensis, Streptomyces rimosus, Streptomyces spectabilis, Streptomyces verticillus, Streptomyces venezuelae, Streptomyces violaceoniger, Streptomyces violaceoruber, Streptomyces mobaraensis, Streptomyces peuceticus, Streptomyces coeruleorubidus, Actinomyces bovis, Actinomyces bowdenii, Actinomyces canis, Actinomyces catuli, Actinomyces coleocanis, Actinomyces europaeus, Actinomyces funkei, Actinomyces georgiae, Actinomyces hongkongensis, Actinomyces israelii, Actinomyces marimammalium, Actinomyces meyeri, Actinomyces oricola, Actinomyces slackii, Actinomyces streptomycini, Actinomyces suis, Actinoplanes teichomyceticus, Actinosynnema pretiosum, Pseudonocardia autotrophica, Amycolatopsis mediteranei, Amycolatopsis orientalis, Saccharopolyspora eritharaea, Saccharopoylspora hirsuta, Pseudomonas aeruginosa, Pseudomonas putida, Pseudomonas syringae, Xantomonas orysae, Burkholderia cenocepacia, Bacillus subtilis, Bacilllus lichenformis, Bacillus megaterium, Mixococcus xantus, Lactobacillus acidophilus, Lactobacillus casei, Lactobacillus brevis, Lactococcus lactis, Brevibacterium casei, Brevibacterium oxydans, Bifidobacterium bifidum, Bifidobacterum longum, Commamonas sp., Corynebacterium glutamicum, Rhodococcus sp., Serratia marescens, Rhizobium trifolii, Rhizobium radiobacter, Sporangium cellulosum, Chondromyces crocatus, Gluconobacter oxydans, Micromonospora grisea, Micrococcus sp., Micromonospora rhodorangea, Micromonospora zionensis, Micromonospora purpurea, Micromonospora inyoensis, Micromonospora sagamiensis, Palnomonospora parontospora, Dactylosporangium matsuzakiense. Nocardia lactamdurans, Bacillus colistinus, Planobispora rosea, Streptomyces arenae, Streptomyces antibioticus, Streptomyces kasugaensis, Streptomyces mitakaensis, Streptomyces bikiniensis, Streptomyces alboniger, Streptomyces tenebrarius, Bacillus circulans, Streptomyces ribosidificus, Bacillus colistinus, Streptomyces tenjimariensis, and Streptomyces pactum.

In some embodiments, the donor bacterial cell of the present invention is non-pathogenic. The donor bacterial cell can be indeed any one of thousands of non-pathogenic bacteria associated with the body of warm-blooded animals, including humans. Preferably, non-pathogenic bacteria that colonize the non-sterile parts of the body (e.g., skin, digestive tract, urogenital region, mouth, nasal passages, throat and upper airway, ears and eyes) are utilized as donor bacterial cells, and the methods of the invention are used to treat bacterial infections of these parts of the body. Examples of particularly preferred donor bacterial species include, but are not limited to: (1) non-pathogenic strains of Escherichia coli (E. coli F18 and E. coli strain Nissle 1917), (2) various species of Lactobacillus (such as L. casei, L. plantarum, L. paracasei, L. acidophilus, L. fermentum, L. zeae and L. gasseri), (3) other nonpathogenic or probiotic skin—or GI colonizing bacteria such as Lactococcus, Bifidobacteria, Eubacteria, and (4) bacterial mini-cells, which are anucleoid cells destined to die but still capable of transferring plasmids (see; e.g., Adler et al (1970) Proc. Nat. Acad, Sci USA 57; 321-326; Frazer et al. (1975) Current Topics in Microbiology and Immunology 69: 1-84; U.S. Pat. No. 4,968,619 to Curtiss III).

In some embodiments, the donor bacterial cell is a probiotic cell. In some embodiments, the probiotic cell of the present invention is a viable probiotic cell. In some embodiments, the probiotic bacterial stain of the present invention is selected from food grade bacteria i.e. bacteria that are used and generally regarded as safe for use in food. In some embodiments, the probiotic bacterial strain is a gram negative strain. In some embodiments, the probiotic bacterial strain is a member of the family of Enterobacteriaceaae. In some embodiments, the probiotic bacterial strain is an E. coli strain. In some embodiments probiotic E. coli strains for use according to the teachings of present invention include non-pathogenic E. coli strains which exert probiotic activity. Example of probiotic E. coli strain is the probiotic Escherichia coli strain BU-230-98, ATCC Deposit No. 202226 (DSM 12799), which is an isolate of the known, commercially available, probiotic Escherichia coli strain M-17. Example of a non-pathogenic is E. coli Nissle 1917. An example of E. coli strain which was not known as probiotic is the laboratory E. coli strain K12.

The plasmid of the present invention can be introduced into the donor bacterial cell by any method known to those of skill in the art. Exemplary methods of transformation include transformation via electroporation of competent cells, passive uptake by competent cells, chemical transformation of competent cells, as well as any other electrical, chemical, physical (mechanical) and/or biological mechanism that results in the introduction of nucleic acid into a cell, including any combination thereof. Procedures for transforming prokaryotic organisms are well known and routine in the art and are described throughout the literature (See, for example, Jiang et al. 2013. Nat. Biotechnol. 31:233-239; Ran et al. Nature Protocols 8:2281-2308 (2013))

Methods:

The present invention also relates to a method for killing a plurality of recipient bacterial cells, comprising exposing said plurality of recipient bacterial cells to a plurality of donor bacterial cells that comprise a copy number of the Targeted-Antibacterial-Plasmid of the present invention that is configured to express a nuclease and one or more guide RNA molecules in said plurality of recipient bacterial cells wherein transfer and expression of said nuclease and guide RNA molecules in said plurality of recipient bacterial cells is lethal for said plurality of recipient bacterial cells.

Typically, the recipient bacterial cells are pathogenic bacteria dispersed throughout the body of a subject, but particularly on the skin or in the digestive tract, urogenital region, mouth, nasal passages, throat and upper airways, eyes and ears. Of particular interest for targeting and eradication are pathogenic strains of Pseudomonas aeruginosa, Escherichia coli, Staphylococcus pneumoniae and other species, Enterobacter spp., Enterococcus spp. and Mycobacterium tuberculosis. Others are also discussed herein, and still others will be readily apparent to those of skill in the art but typically include Clostridium difficile, Escherichia coli, Clostridium tetani, Helicobacter pylori, Fusobacterium nucleatum, Gardnerella vaginitis, Porphyromonas gingivalis, Aggregatibacter actinomycetemcomitans, Listeria monocytogenes, Staphylococcus aureus, Campylobacter jejuni, Vibrio vulnificus, Salmonella typhi, Clostridium botulinum, Mycobacterium tuberculosis, Mycobacterium leprae, Mycobacterium lepromatosis, Corynebacterium diptheriae, Klebsiella pneumoniae, Acinetobacter baumannii, Streptococcus mutans, group B streptococci, including but not limited to, Staphylococcus aureus, Streptococcus agalactiae, Streptococcus pneumonia, Enterococcus spp. including, but not limited to, Enterococcus faecalis.

The method of the present invention is particularly suitable for treating treat a variety of bacterial infections or bacterially related undesirable conditions. In some embodiments, the bacterial infection is an infection of the gastrointestinal tract, an infection of the urogenital tract, an infection of the respiratory tract, like, for example rhinitis, tonsillitis, pharyngitis, bronchitis, pneumonia, an infection of the inner organs, like, for example, nephritis, hepatitis, peritonitis, endocarditis, meningitis, osteomyelitis, an infection of the eyes, the ears as well as a cutaneous and a subcutaneous infection, diarrhea, skin disorders, toxic shock syndrome, bacteraemia, sepsis, and tuberculosis. In some embodiments, the infection is caused by gram-positive bacteria or gram-negative bacteria. In some embodiments, the bacterial infection is caused by a bacterium selected from the group consisting of Staphylococcus, Streptococcus, Pseudomonas, Escherichia, Salmonella, Helicobacter, Neisseria, Campylobacter, Chlamydia, Clostridium, Vibrio, Treponema, Mycobacterium, Klebsiella, Actinomyces, Bacterioides, Bordetella, Borrelia, Brucella, Corynebacterium, Diplococcus, Enterobacter, Fusobacterium, Leptospira, Listeria, Pasteurella, Proteus, Rickettsia, Shigella, Sphaerophorus, Yersinia, or combinations thereof. Examples of bacteria which cause bacterial respiratory tract infections include, but not limited to, Chlamydia species (e.g., Chlamydia pneumoniae), Klebsiella species, Haemophilus influenzae, Legionallaceae family, mycobacteria (e.g., Mycobacterium tuberculosis), Pasteurellaceae family, Pseudomonas species, Staphylococcus (e.g., methicillin resistant Staphylococcus aureus and Staphylococcus pyrogenes), Streptococcus (e.g., Streptococcus enteritidis, Streptococcus Fasciae, and Streptococcus pneumoniae). Examples of bacteria which cause intestinal infections include, but not limited to Bacteroidaceae family, Campylobacter species, Chlamydia species, Clostridium, Enterobacteriaceae family (e.g., Citrobacter species, Edwardsiella, Enterobacter aerogenes, Escherichia coli, Klebsiella species, Salmonella species, and Shigella flexneri), Gardinella family, Listeria species, Pasteurellaceae family, Pseudomonas species, Streptococcus (e.g., Streptococcus enteritidis, Streptococcus Fasciae, and Streptococcus pneumoniae), Helicobacter Family. Other representative examples of these uses include treatment of (1) conjunctivitis, caused by Haemophilus sp., and corneal ulcers, caused by Pseudomonas aeruginosa; (2) otititis extema, caused by Pseudomonas aeruginosa; (3) chronic sinusitis, caused by many Gram-positive cocci and Gram-negative rods; (4) cystic fibrosis, associated with Pseudomonas aeruginosa; (5) and Enteritis, caused by Helicobacter pylori (ulcers), Escherichia coli, Salmonella typhimurium, Campylobacter and Shigella sp.; (6) open wounds, both surgical and non-surgical, as a prophylactic measure for many species; (7) burns to eliminate Pseudomonas aeruginosa or other Gram-negative pathogens; (8) acne, caused by Propionobacter acnes; (9) nose and skin infections caused by methicillin resistant Staphylococcus aureus (MSRA); (10) body odor caused mainly by Gram-positive anaerobic bacteria (i.e., use in deodorants); (11) bacterial vaginosis associated with Gardnerella vaginalis and other anaerobes; and (12) gingivitis and/or tooth decay caused by various organisms.

The present invention also relates to use of a donor bacterial cell of the present invention for use in a method for enhancing the clinical efficacy of an antibiotic for clearing an infection in a subject in need thereof, i.e. meaning that the donor bacterial cell of the invention improves clearance of the bacteria and recovery from the infection compared to the standalone antibiotic treatment.

A further object of the present invention relates to method of treating an infection in a subject caused by a bacterial cell comprising an antibiotic resistance gene, in which the method comprises administering to the subject a therapeutically effective amount of the antibiotic in combination with a therapeutically effective amount of bacterial donor cells comprising a copy number of targeted-antibacterial-plasmids that encodes for one or more guide RNA molecule suitable for targeting the antibiotic resistance gene, thereby inactivating the antibiotic resistance gene and sensitizing the bacterial cell to said antibiotic.

In particular, the combination of the antibiotic with the donor bacterial cell that targets one or more antibiotic resistance gene(s) can enhance the capacity of host to repair the damages induced by infection before starting treatment. The combination of the invention could thus decrease the morbidity. According to the invention, the donor bacterial cell targets one or more antibiotic resistance gene(s) potentiates the activity of the antibiotic for the clearance of the bacterial infection. The term “potentiate”, as used herein, means to enhance or increase at least one biological effect or activity of the antibiotic so that either (i) a given concentration or amount of the antibiotic results in a greater biological effect or activity when the antibiotic is potentiated than the biological effect or activity that would result from the same concentration or amount of the antibiotic when not potentiated; or (ii) a lower concentration or amount of the antibiotic is required to achieve a particular biological effect or activity when the antibiotic is potentiated than when the antibiotic is not potentiated; or (iii) both (i) and (ii). In particular, the donor bacterial cells combined to antibiotics: -1- may impact on a specific niche that is not accessible to antibiotic (i.e. intracellular compartments of some cells), allowing antibiotics to work when combined to donor bacterial cells; -2- may impact on the recovery phase from antibiotic treatment of infection (tissue repair, restoration of physiological function of infected tissues); -3- may limit side effects (for example alteration of microbiota composition in gut, skin, respiratory tract . . . ) and -4- may restrain the development of antibiotic resistance (often due to acquisition of resistance by the microbiota bacteria).

In some embodiments, the antibiotic is selected from the group consisting of aminoglycosides, beta-lactams, quinolones or fluoroquinolones, macrolides, sulfonamides, sulfamethaxozoles, tetracyclines, streptogramins, oxazolidinones (such as linezolid), rifamycins, glycopeptides, polymixins, lipo-peptide antibiotics. In particular, the antibiotic is selected among beta-lactams. All members of beta-lactams possess a beta-lactam ring and a carboxyl group, resulting in 55 similarities in both their pharmacokinetics and mechanism of action. The majority of clinically useful beta-lactams belong to either the penicillin group or the cephalosporin group, including cefamycins and oxacephems. The beta-lactams also include the carbapenems and monobactams. Generally speaking, beta-lactams inhibit bacterial cell wall synthesis. More specifically, these antibiotics cause ‘nicks’ in the peptidoglycan net of the cell wall that allow the bacterial protoplasm to flow from its protective net into the surrounding hypotonic medium. Fluid then accumulates in the naked 65 protoplast (a cell devoid of its wall), and it eventually bursts, leading to death of the organism. Mechanistically, beta-lactams act by inhibiting D-alanyl-D-alanine transpeptidase activity by forming stable esters with the carboxyl of the open lactam ring attached to the hydroxyl group of the enzyme target site. Beta-lactams are extremely effective and typically are of low toxicity. As a group, these drugs are active against many gram-positive, gram-negative and anaerobic organisms. Drugs falling into this category include 2-(3-alanyl)clavam, 2-hydroxymethylclavam, 7-methoxycephalosporin, epi-thienamycin, acetyl-thienamycin, amoxicillin, apalcillin, aspoxicillin, azidocillin, azlocillin, aztreonam, bacampicillin, blapenem, carbenicillin, carfecillin, carindacillin, carpetimycin A and B, cefacetril, cefaclor, cefadroxil, cefalexin, cefaloglycin, cefaloridine, cefalotin, cefamandole, cefapirin, cefatrizine, cefazedone, cefazolin, cefbuperazone, cefcapene, cefdinir, cefditoren, cefepime, cefetamet, cefixime, cefinenoxime, cefinetazole, cefminox, cefmolexin, cefodizime, cefonicid, cefoperazone, ceforamide, cefoselis, cefotaxime, cefotetan, cefotiam, cefoxitin, cefozopran, cefpiramide, cefpirome, cefpodoxime, cefprozil, cefquinome, cefradine, cefroxadine, cefsulodin, ceftazidime, cefteram, ceftezole, ceftibuten, ceftizoxime, ceftriaxone, cefuroxime, cephalosporin C, cephamycin A, cephamycin C, cephalothin, chitinovorin A, chitinovorin B, chitinovorin C, ciclacillin, clometocillin, cloxacillin, cycloserine, deoxy pluracidomycin B and C, dicloxacillin, dihydro pluracidomycin C, epicillin, epithienamycin D, E, and F, ertapenem, faropenem, flomoxef, flucloxacillin, hetacillin, imipenem, lenampicillin, loracarbef, mecillinam, meropenem, metampicillin, meticillin (also referred to as methicillin), mezlocillin, moxalactam, nafcillin, northienamycin, oxacillin, panipenem, penamecillin, penicillin G, N, and V, phenethicillin, piperacillin, povampicillin, pivcefalexin, povmecillinam, pivmecillinam, pluracidomycin B, C, and D, propicillin, sarmoxicillin, sulbactam, sultamicillin, talampicillin, temocillin, terconazole, thienamycin, andticarcillin. More particularly, Carbapenems include for instance the approved antibiotics Imipenem, Meropenem, Ertapenem, Doripenem, Panipenem/betamipron, Biapenem and the experimental antibiotics Razupenem, Tebipenem, Lenapenem and Tomopenem.

Once the donor bacterial cells are produced, they are used to protect against one or more selected pathogens in individuals requiring such treatment. Depending on the cell population or tissue targeted for protection, the following modes of administration of the donor bacterial cells of the invention are contemplated: topical, oral, nasal, pulmonary/bronchial (e.g., via an inhaler), ophthalmic, rectal, urogenital, subcutaneous, intraperitoneal and intravenous. The donor bacterial cells preferably are supplied as a pharmaceutical preparation, in a delivery vehicle suitable for the mode of administration selected for the patient being treated.

For instance, to deliver the donor bacterial cells to the gastrointestinal tract or to the nasal passages, the preferred mode of administration is by oral ingestion or nasal aerosol, or by feeding (alone or incorporated into the subject's feed or food). In some embodiments, the donor bacterial cells of the present invention can be supplied as a powdered, lyophilized preparation in a gel capsule, or in bulk for sprinkling into food or beverages. For topical applications, the donor bacterial cell may be formulated as an ointment or cream to be spread on the affected skin surface. Ointment or cream formulations are also suitable for rectal or vaginal delivery, along with other standard formulations, such as suppositories. The appropriate formulations for topical, vaginal or rectal administration are well known to medicinal chemists.

Other uses for the donor bacterial cell of the invention are also contemplated. These include agricultural and horticultural applications, such as: (1) use on meat or other foods to eliminate pathogenic bacteria; (2) use in animal feed (chickens, cattle) to reduce bio-burden or to reduce or eliminate particular pathogenic organisms (e.g., Salmonella); (3) use on fish to prevent “fishy odor” caused by Proteus and other organisms; and (4) use on cut flowers to prevent wilting.

Compositions:

A further object of the present invention relates to a composition comprising an amount of the donor bacterial cells of the present invention.

In some embodiments, the composition of the present invention is a pharmaceutical composition. Pharmaceutical preparations comprising the donor bacteria cells are formulated in dosage unit form for ease of administration and uniformity of dosage. Dosage unit form, as used herein, refers to a physically discrete unit of the pharmaceutical preparation appropriate for the patient undergoing treatment. Each dosage should contain a quantity of the donor bacteria calculated to produce the desired antibacterial effect in association with the selected pharmaceutical carrier. Procedures for determining the appropriate dosage unit are well known to those skilled in the art. Dosage units may be proportionately increased or decreased based on the weight of the patient. Appropriate concentrations for achieving eradication of pathogenic bacteria in a target cell population or tissue may be determined by dosage concentration curve calculations, as known in the art. Pharmaceutical compositions of the present invention for administration topically can be in a form suitable for topical use such as, for example, an aerosol, cream, ointment, lotion or dusting powder. Pharmaceutical compositions provided herein may be formulated for administration by inhalation. For example, the compositions may be in a form as an aerosol, a mist or a powder. Thus compositions described herein may be delivered in the form of an aerosol spray presentation from pressurised packs or a nebuliser, with the use of a suitable propellant such as for example dichlorodifluoromethane, trichlorofluoromethane, dichlorotetrafluoroethane, carbon dioxide or other suitable gas. Where using a pressurized aerosol, a dosage unit may be determined by providing a valve to deliver a metered amount.

In some embodiments, the donor bacterial cell of the present invention is encapsulated in order to be protected against the stomach when the donor bacterial cell is administered to the subject by ingestion (i.e. oral route). Accordingly, in some embodiments the donor bacterial cell of the present invention is formulated in compositions in an encapsulated form so as significantly to improve their survival time. In such a case, the presence of a capsule may in particular delay or prevent the degradation of the microorganism in the gastrointestinal tract. It will be appreciated that the compositions of the present embodiments can be encapsulated into an enterically-coated, time-released capsule or tablet. The enteric coating allows the capsule/tablet to remain intact (i.e., undissolved) as it passes through the gastrointestinal tract, until such time as it reaches the small intestine. Methods of encapsulating live bacterial cells are well known in the art (see, e.g., U.S. patents to General Mills Inc. such as U.S. Pat. No. 6,723,358). For example, micro-encapsulation with alginate and Hi-Maize™ starch followed by freeze-drying has been proved successful in prolonging shelf-life of bacterial cells in dairy products [see, e.g., Kailasapathy et al. Curr Issues Intest Microbiol. 2002 September; 3(2):39-48]. Alternatively encapsulation can be done with glucomannane fibers such as those extracted from Amorphophallus konjac. Alternatively, entrapment of viable probiotic in sesame oil emulsions may also be used [see, e.g., Hou et al. J. Dairy Sci. 86:424-428]. In some embodiments, agents for enteric coatings are preferably methacrylic acid-alkyl acrylate copolymers, such as Eudragit® polymers. Poly(meth)acrylates have proven particularly suitable as coating materials. EUDRAGIT® is the trade name for copolymers derived from esters of acrylic and methacrylic acid, whose properties are determined by functional groups. The individual EUDRAGIT® grades differ in their proportion of neutral, alkaline or acid groups and thus in terms of physicochemical properties. The skillful use and combination of different EUDRAGIT® polymers offers ideal solutions for controlled drug release in various pharmaceutical and technical applications. EUDRAGIT® provides functional films for sustained-release tablet and pellet coatings. The polymers are described in international pharmacopeias such as Ph.Eur., USP/NF, DMF and JPE. EUDRAGIT® polymers can provide the following possibilities for controlled drug release: gastrointestinal tract targeting (gastroresistance, release in the colon), protective coatings (taste and odor masking, protection against moisture) and delayed drug release (sustained-release formulations). EUDRAGIT® polymers are available in a wide range of different concentrations and physical forms, including aqueous solutions, aqueous dispersion, organic solutions, and solid substances. The pharmaceutical properties of EUDRAGIT® polymers are determined by the chemical properties of their functional groups.

In some embodiments, the donor bacterial cell of the present invention is administered to the subject in the form of a food composition. Accordingly one further aspect of the present invention relates to a food composition comprising an amount of the donor bacterial cell of the present invention. In some embodiments, the food composition that comprises the donor bacterial cell of the present invention is selected from complete food compositions, food supplements, nutraceutical compositions, and the like. The composition of the present invention may be used as a food ingredient and/or feed ingredient. In some embodiments, the food composition that comprises the donor bacterial cell of the present invention contains at least one prebiotic. “Prebiotic” means food substances intended to promote the growth of the donor bacterial cell of the present invention in the intestines. The prebiotic may be selected from the group consisting of oligosaccharides and optionally contains fructose, galactose, mannose, soy and/or inulin; and/or dietary fibers.

The composition that comprises the donor bacterial cell of the present invention typically comprises carriers or vehicles. “Carriers” or “vehicles” mean materials suitable for administration and include any such material known in the art such as, for example, any liquid, gel, solvent, liquid diluent, solubilizer, or the like, which is non-toxic and which does not interact with any components of the composition in a deleterious manner. Examples of acceptable carriers include, for example, water, salt solutions, alcohol, silicone, waxes, petroleum jelly, vegetable oils, polyethylene glycols, propylene glycol, liposomes, sugars, gelatin, lactose, amylose, magnesium stearate, talc, surfactants, silicic acid, viscous paraffin, perfume oil, fatty acid monoglycerides and diglycerides, petroethral fatty acid esters, hydroxymethyl-cellulose, polyvinylpyrrolidone, and the like. In some embodiments, the composition that comprises the donor bacterial cell of the present invention contains protective hydrocolloids (such as gums, proteins, modified starches), binders, film forming agents, encapsulating agents/materials, wall/shell materials, matrix compounds, coatings, emulsifiers, surface active agents, solubilizing agents (oils, fats, waxes, lecithins etc.), adsorbents, carriers, fillers, co-compounds, dispersing agents, wetting agents, processing aids (solvents), flowing agents, taste masking agents, weighting agents, jellifying agents, gel forming agents, antioxidants and antimicrobials. The composition may also contain conventional pharmaceutical additives and adjuvants, excipients and diluents, including, but not limited to, water, gelatine of any origin, vegetable gums, ligninsulfonate, talc, sugars, starch, gum arabic, vegetable oils, polyalkylene glycols, flavouring agents, preservatives, stabilizers, emulsifying agents, buffers, lubricants, colorants, wetting agents, fillers, and the like. In all cases, such further components will be selected having regard to their suitability for the intended recipient.

The invention will be further illustrated by the following figures and examples. However, these examples and figures should not be interpreted in any way as limiting the scope of the present invention.

### FIGURES

FIG. 1. Transfer of TAP by F plasmid machinery mediates killing of a targeted E. coli strain. (a) TAPs modules consist of CRISPR system composed of wild type (cas9) or catalytically dead cas9 (dcas9) genes expressed from the weak constitutive BBa_J23107 promoter and a gRNA module expressed from the strong constitutive BBa_J23119 promoter; the F plasmid origin of transfer (oriT_{F}); the pBBR1 origin of replication (oriV), and a set of resistance cassettes (Ap, ampicillin; Kn, kanamycin; Cm, chloramphenicol; St, streptomycin; Gm, gentamycin), an optional cassette acrying the sfgfp or mcherry genes highly expressed from the broad-host range synthetic BioFab promoter (b) Diagram of the TAP antibacterial strategy. A donor strain produces the F plasmid conjugation machinery to transfer the TAP into the recipient strain. Targeted recipient carries sequence recognized by CRISPR(i) system that induces killing or gene expression inhibition. Non-targeted recipient lacking the spacer recognition sequence are insensitive to CRISPR(i) activity. (c) Histogram of TAPs transfer estimated by flow cytometry show that TAPₖₙ-Cas9-nsp-GFP and TAPₖₙ-Cas9-lacZ2-GFP are transferred with similar efficiency in recipient cells after 3 h of mating. Donors TAP-Cas9-nsp (LY1371) or TAP-Cas9-lacZ2 (LY1380), recipient HU-mCherry lac+ (LY248). (d) Histograms of the concentration of viable transconjugants estimated by plating assays show viability loss associated with the acquisition of TAP-Cas9-lacZ2. The corresponding percentage of viable transconjugants (ratio T/R+T) is shown above each bar. (e) Fold-increase of the recipient population counts over the 3 h of mating. Donors TAP-Cas9-nsp (LY1369) or TAP-Cas9-lacZ2 (LY1370), recipient lac+ (LY827). (c-e) Mean and SD are calculated from 3 independent experiments.

FIG. 2. Real-time visualization of E. coli killing after acquisition of TAP. (a-b) Single-cells time-lapse quantification of transconjugants (a) bacterial and (b) nucleoid lengths. Average and SD are indicated (n cells analysed). The time of TAP acquisition (red dashed line at 0 min) corresponds to a 15% increase in the green fluorescence in the transconjugant cells. (c-d) Single-cells time lapse quantification of transconjugants (c) cell length and (d) skewness of RecA-GFP fluorescence signal. Average and SD are indicated (n cells analysed). The time of TAP acquisition (red dashed line at 0 min) corresponds to a 30% increase in the green fluorescence in the transconjugant cells.

FIG. 3. TAP system specifically kills targeted recipients in a mix of targeted and non-targeted E. coli recipient cells. (a) Viable transconjugant cells and percentage of transconjugants (ratio T/R+T) through TAPₖₙ-Cas9-nsp or TAPₖₙ-Cas9-lacZ2 transfer from donor to a mixed lac+ and lac− recipient population. (b) Quantification of fold-increase in lac+ and lac− recipient populations counts over the 6 h of mating. Mean and SD are calculated from 4 independent experiments. Donors: TAP-Cas9-nsp (LY1369) or TAP-Cas9-lacZ2 (LY1370); recipients lac+ (LY827) and lac− (LY848). (c) Single-cell quantification showing cell length increase in the targeted lac+ transconjugant cells but not non-targeted lac− transconjugants. The time of TAP acquisition (red dashed line at 0 min) corresponds to a 15% increase in the green fluorescence in the transconjugant cells. Cell length average is indicated with SD (n cells analysed). Donor TAP-Cas9-lacZ2 (LY1380); recipients HU-mCherry lac+ (LY248) and DnaN-mCherry lac− (LY1423).

FIG. 4. TAP re-sensitises pOXA48-carrying recipient cells and impedes resistance dissemination. (a) Diagram of the TAP-mediated anti-resistance strategy. TAP-Cas9-OXA48 targeting the bla_{OXA48} promoter is transferred from a donor to an ampicillin resistant recipient cells carrying the pOXA-48a plasmid. Acquisition of the TAP-Cas9-OXA48 induces double-strand-breaks (DSBs) into the plasmid, while the TAP-dCas9-OXA48 inhibits bla_{OXA48} gene expression. Both TAPs sensitise the transconjugant cells to ampicillin. (b) Histograms showing reduction of ampicillin resistance in transconjugants cells after acquisition of the TAPₖₙ-Cas9-OXA48, TAPₖₙ-dCas9-OXA48 and TAPₖₙ-Cas9-OXA48-PemI. Percentages of transconjugants (ratio T/R+T) are indicated. (c) Histograms showing the frequency of donor cells acquiring ampicillin resistance through transfer of pOXA-48a from the recipients (as depicted in the above diagram). (d) Histograms show the frequency of ampicillin-resistance acquisition through pOXA-48a transfer into R #2 plasmid-free wt recipient that have received the TAPs (TAP-transc. #2) (as depicted in the above diagram). Mean and SD are calculated from at least 3 independent experiments. Donors TAP-dCas9-nsp (LY1524), TAP-Cas9-nsp (LY1369), TAP-dCas9-OXA48 (LY1523), TAP-Cas9-OXA48 (LY1522) or TAP-PemI-Cas9-OXA48 (LY1549); Recipients R #1 wt/pOXA-48a (LY1507) and R #2 wt (LY945).

FIG. 5. Efficient and strain-specific killing of TAPs within a multispecies recipient population. (a) Efficiency of TAP-Cas9-nsp transfer from E. coli (LY1369) donor to C. rodentium, E. cloacae, E. coli EPEC or HS recipients. Histograms show the percentages of tranconjugants (T/R+T) after 24 h of conjugation for C. rodentium, E. cloacae, E. coli EPEC recipients and 3 h for E. coli HS recipient; mean and SD are calculated from at least 3 independent experiments. (b) TAPs carrying specific spacers identified with the CSTB algorithm were tested against each recipient cells. To account for the variability of TAP transfer in the different recipient strains, the histograms show the relative abundance of viable transconjugants normalized by viable transconjugants obtained for the TAP_{K}n-Cas9-nsp. Numbers in brackets above asterisk indicate replicates with detection limit of transconjugants below 10⁻⁸. Mean and SD are calculated from 3 independent experiments. (c) Proportion of recipients estimated by plating assay before mating with donors. Mean and SD calculated from 3 independent experiments are indicated for each recipient strains. (d) Each TAP carrying specific spacers were tested through conjugation between E. coli donors and a recipient population containing all recipient species. Histograms show the proportion of viable transconjugants in the mixed population after 3 h of mating. Numbers in brackets above asterisk indicate replicates with detection limit of transconjugants below 10⁻⁸. Mean and SD are calculated from 3 independent experiments. Donors TAP-Cas9-nsp (LY1369), TAP-Cas9-Cr1 (LY1597), TAP-Cas9-Ec1 (LY1566), TAP-Cas9-EPEC (LY1618), TAP-Cas9-EEC (LY1665); recipients C. rodentium (LY720), E. cloacae (LY1410), E. coli EPEC (LY1615) or HS (LY1601).

### EXAMPLE

Methods:

Bacterial Strains, Plasmids, Primer and Growth Culture Conditions.

Bacterial strains construction and growth procedures. Plasmid cloning were done by Gibson Assembly (Gibson et al., 2009) and verified by Sanger sequencing (Eurofins Genomics). Chromosome mutation were transferred by phage P1 transduction to generate the final strains. Strains were grown at 37° C. in Luria-Bertani (LB) broth, M9 medium supplemented with glucose (0.2%) and casamino acid (0.4%) (M9-CASA) or M63 medium supplemented with glucose (0.2%) and casamino acid (0.4%) (M63). When appropriate, the media were supplemented with the following antibiotics: 50 μg/ml kanamycin (Kan), 20 μg/ml chloramphenicol (Cm), 10 μg/ml tetracycline (Tc), 20 μg/ml nalidixic acid (Nal), 20 μg/ml streptomycin (St), 100 μg/ml ampicillin (Ap), 10 μg/ml gentamycin (Gm), 50 μg/ml rifampicin (Rif). When appropriate 40 μg/ml 5-bromo-4-chloro-3-indolyl-β-d-galactopyranoside (X-Gal) and 40 μM isopropyl β-D-1-thiogalactopyranoside (IPTG) were added for screening of LAC phenotype.

TAPs construction and one-step-cloning change of the spacer sequence on the TAPs. Plasmid construction was performed by IVA cloning (Garcia-Nafria et al., 2016), expect for changing the spacer sequence in the TAPs, which was performed by the replacement of the spacer in pEGL129 by a SapI-spacer-SapI DNA sequence. The nsp (non-specific) spacer sequence is flanked by two SapI restriction sites that allow for liberation of non-cohesive DNA ends upon SapI digestion. To replace the nsp spacer, a new spacer is constructed by annealing 2 oligonucleotides with complementary sequences to the non-cohesive ends generated by SapI restriction of TAP-Cas9-nsp or TAP-dCas9-nsp plasmids. Ligation production between the new spacer fragment and the TAP backbone was transformed into DH5α or TB28 strains. Constructions were verified by PCR reaction and sequencing.

Congo Red Assay

Curli production colony assay for. E. coli strain OmpR234 with or without plasmids were plated on Congo Red medium (10 g bacto tryptone, 5 g yeast extract, 18 g bacto agar, 40 μg/ml Congo Red and 20 μg/ml Coomassie Brilliant blue G) and incubated 4 days at 30° C. Colonies were visualized at ×10 magnification with a M80 stereomicroscope (Leica). Digital images were captured with an IC80-HD integrated camera coupled to the stereomicroscope, operated via LASv4.8 software (Leica).

Liquid aggregation test. Overnight culture of E. coli strain OmpR234 with or without plasmids were diluted to an A₆₀₀ of 0.05 in 1 ml M9-CASA medium supplemented with 25 μg/ml of Congo Red. Culture were grown without agitation at 30° C. for 24 h and image captured.

Conjugation Assay

Overnight cultures grown in LB of donor and recipient strains were diluted to an A₆₀₀ of 0.05 and grown until an A₆₀₀ comprised between 0.7 and 0.9 was reached. 50 μl of donor and 150 μl of recipient cultures were mixed into an Eppendorf tube to obtain a 1:3 donor to recipient ratio. At time 0 min, 100 μl of the mix were diluted into 1 ml LB, serial diluted and plated on LB agar supplemented with antibiotics selecting for donor, recipient and transconjugant cells. The remaining 100 μl were incubated for 1 h30 at 37° C. 1 ml of LB was added gently and the tubes were incubated at 37° C. for another 1 h30, 4 h30 or 22 h30. Conjugation mix were then vortexed, serial diluted and plated as for time 0 min.

Long term conjugation experiments. Conjugation mixes were prepared and incubated at 37° C. without agitation. Every 24 h, 100 μl of the mix were diluted into 1 ml of LB and re-incubated at 37° C. The remaining of the mixes were vortexed, serial diluted and plated on LB agar supplemented with antibiotics selecting for donor, recipient and transconjugant cells. At day 1 and day 7, 100 clones of the resulting ampicillin resistant recipients mixed with the RAP-dCas9-OXA48 carrying donor were streaked on LB agar supplemented with Tc or Kn to evaluate the presence of the F-Tn10 or TAP-dCas9-OXA48 plasmids respectively.

Multispecies conjugation. Overnight cultures grown in LB of donor and recipient strains were diluted to an A₆₀₀ of 0.05 and grown until an A₆₀₀ comprised between 0.7 and 0.9 was reached. A recipient mix is prepared by mixing C. rodentium, E. cloacae, E. coli EPEC and E. coli HS recipients strains in equal proportion. This mix is serial diluted and plated on LB agar supplemented with antibiotics to select for each recipient. 100 μl of donor and 100 μl of the recipient mix were added to an Eppendorf tube to perform mating. At time 0 min, 100 μl of the mix were diluted into 1 ml LB, serial diluted and plated on LB agar supplemented with antibiotics to select for donor, recipients and transconjugants. The remaining 100 μl were incubated for 1 h30 at 37° C. 1 ml of LB was gently added and the tubes were incubated for an additional 1 h30 at 37° C. Conjugation mix were then vortexed, serial diluted and plated on LB agar supplemented with antibiotics to select for donor, recipients and transconjugants. In the figures, the efficiencies of conjugation are represented either as the final concentration of transconjugant cell (CFU/ml) or as the percentage of transconjugant cells calculated from the ratio (T/R+T).

Transformation Assay

Overnight cultures grown in LB were 1/100 diluted and grown until an A₆₀₀ comprised between 0.4 and 0.6. Cells were treated with Rubidium Chloride and 90 μl of the resulting competent cells transformed with 100 ng of plasmid and heat shock. Following the 1 h incubation at 37° C. for phenotypic expression, cells were centrifugated 5 min at 5000 rpm, resuspended in 100 μl of LB, and 10 μl of serial dilution were spotted on LB plates supplemented with the appropriate antibiotics.

Live-Cell Microscopy Imaging and Analysis

Time-lapse experiments. Overnight cultures in M9-CASA (between E. coli) or M63 (between E. coli and C. rodentium) of donor and recipient cells were diluted to an A₆₀₀ of 0.05 and grown until an A₆₀₀ comprised between 0.7 and 0.9. 25 μl of donor and 75 μl of recipient were mixed into an Eppendorf tube and 50 μl of the mix was loaded into a B04A microfluidic chamber (ONIX, CellASIC®). Nutrient supply was maintained at 1 psi and the temperature maintained at 37° C. throughout the imaging process. Cells were imaged every 10 min for 3 h.

Image acquisition. Conventional wide-field fluorescence microscopy imaging was carried out on an Eclipse Ti-E microscope (Nikon), equipped with ×100/1.45 oil Plan Apo Lambda phase objective, FLash4 V2 CMOS camera (Hamamatsu), and using NIS software for image acquisition. Acquisition were performed using 50% power of a Fluo LED Spectra X light source at 488 nm and 560 nm excitation wavelengths. Exposure settings were 50 ms for sfGFP and 50 ms for mCherry produced from the TAPs; 100 ms for RecA-GFP; 100 ms for HU-mCherry; 100 ms for DnaN-mCherry.

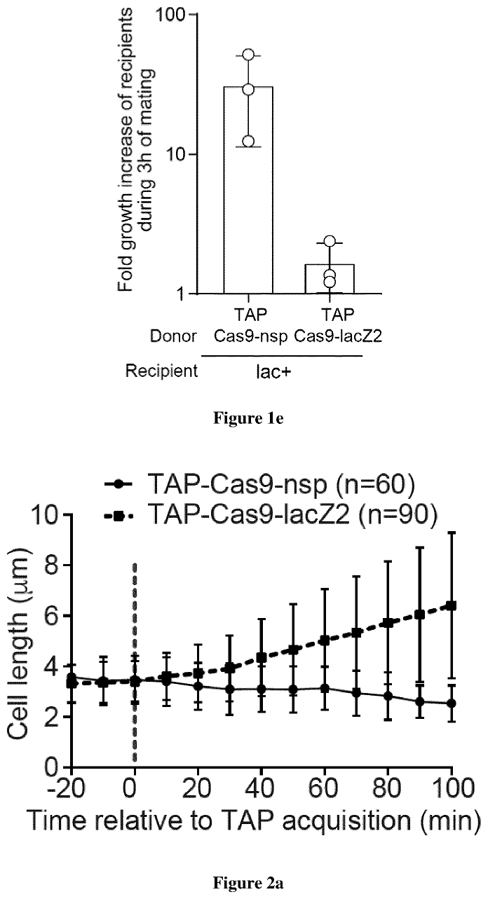

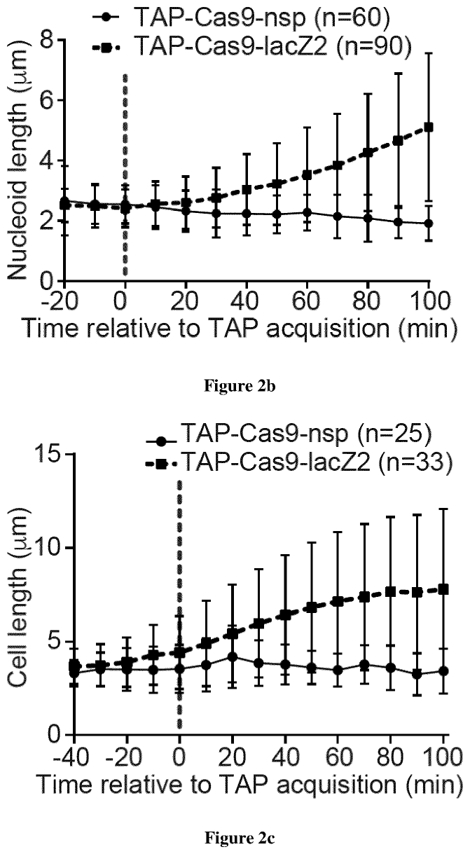

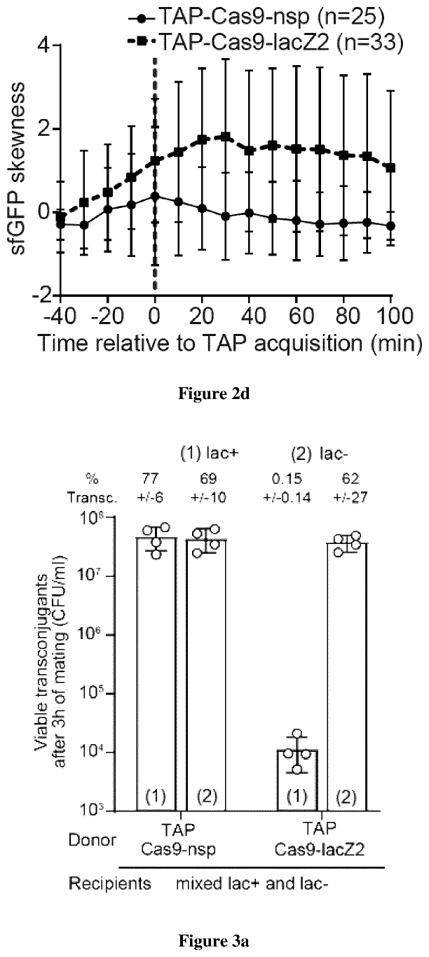

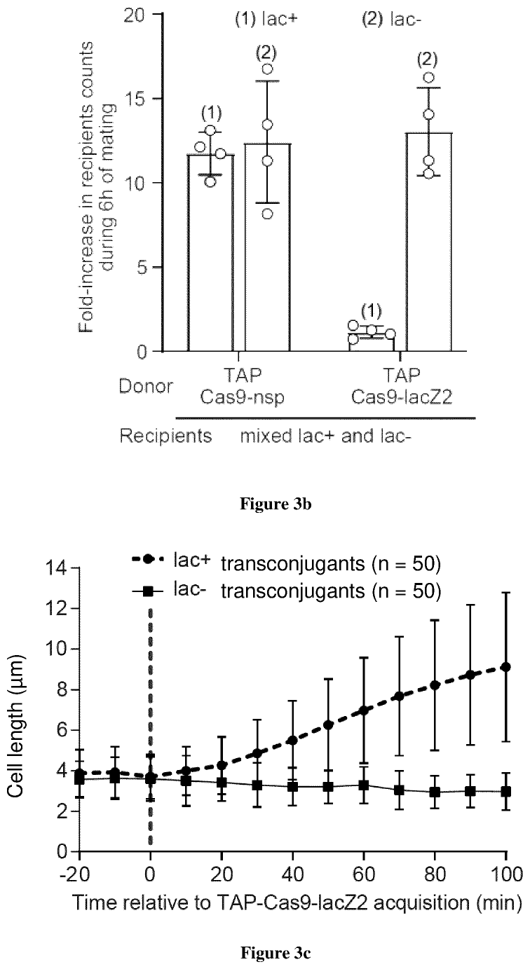

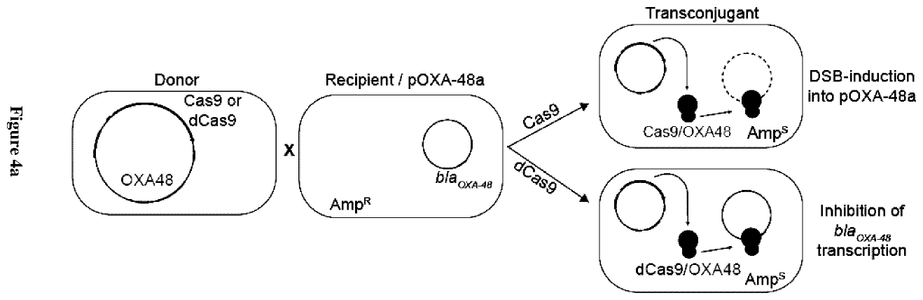

Image analysis. Quantitative image analysis was done using Fiji software with MicrobeJ plugin (Ducret et al., 2016). The Manual-editing interface of MicrobeJ was used to optimize cell detection and the Mean intensity fluorescence, skewness and cell length parameters were automatically extracted and plotted. We defined the timing of TAP acquisition (time t=0) by analyzing the increase of the fluorescence signal conferred by the TAPs (sfGFP or mCh). Plasmid acquisition was validated when a 15% sfGFP or a 30% mCherry fluorescence increase was observed in the transconjugant cells. Fluorescence profiles of each cells were then aligned according the defined t=0 to generate the graphs presented in FIGS. 2a, 2b, 2c, 2d, 3c.

Flow Cytometry

Conjugation was done as described in the conjugation assay section in 0.1 μm filtered LB. At time 90 min and 180 min, conjugation mix were diluted to an A₆₀₀ of 0.03 in 0.1 μm filtered LB and analysed into an Attune NxT acoustic focusing cytometer at a 25 μl/min flow rate. Forward scattered (FSC), Side scattered (SSC) as well as fluorescence signal BL1 (sfGFP) and YL2 (mCherry) were acquired with the appropriate PMT setting and represented with the Attunem NxT analysis software. To verify the absence of toxicity of the Cas9 or dCas9 constitutive expression from the TAPs, we compared the growth of E. coli MS388/TAP with the cas9 or dcas9 or without any cas9 gene. Those strains were grown overnight in 0.1 μm filtered LB and diluted to an A₆₀₀ of 0.05 in 0.1 μm filtered LB. They were grown during 8 h and the A₆₀₀ and CFU/mL were estimated by plating assays at 0, 2, 4, 6 and 8 hours. In parallel, at 1 h, 2 h and 5 h30 the strains were analysed into the Attune NxT acoustic Focusing cytometer at a 25 μl/min flow rate. Forward scattered (FSC) was acquired and represented with the Attunem NxT software.

Analysis of TAP-Escape Mutants

In E. coli. The 31 TAP-escape mutants were streaked on medium supplemented with X-Gal and IPTG to determine their LAC phenotype. TAP-escape mutants exhibiting lac+ phenotype were classified as “Blue” and the others as “White”. To determine the acquisition of point mutation or deletion that modify the targeted lacZ2 locus, a PCR was realized with OL240 and OL654 that amplify a fragment of 748pb encompassing the lacZ2 locus in wt strain. For escape mutants that exhibited no deletion of the lacZ2 locus but still had an active TAP CRISPR system, the PCR product was sequenced and the mutations identified. A PCR was also done with OL655 and OL656 to amplify a larger fragment around lacZ2 and observe large deletion as previously described (Cui and Bikard, 2016). To determine the activity of the TAPs extracted from escape mutants, conjugation was performed between the TAP-escape mutants and an E. coli MS388 lac+ strain as described in the conjugation assays section. In parallel, the activity of the TAPs extracted with the Machery Nagel NucleoSpin® Plasmid kit from escape mutants were verified by transformation into lac+ and lac− strains as described in the transformation assay section. Seven inactive TAPs were sequenced to identify mutations inactivating CRISPR system. C. rodentium. For the 20 TAP-Cas9-Cr1-escape mutants, a PCR with OL686 and OL687 was performed to determine deletions in the chromosome locus. To verify the CRISPR activity of the TAPs from C. rodentium TAP-escape mutants, conjugation was performed during 5 hours between the C. rodentium mutants and the E. coli MS388 strain to generate new E. coli TAPs donors. Then conjugation was performed during 24 h between those new donors and fresh C. rodentium recipients and plated to select for recipient and transconjugants. To confirm the activity of the TAPs isolated form C. rodentium escape mutants, TAPs were extracted with Machery Nagel NucleoSpin® Plasmid kit and transformed by electroporation (2.5 kV) into wt C. rodentium cells treated with 10% sucrose. Following 1 h of incubation at 37° C., cells were plated on LB-agar supplemented or not with Kan to evaluate the transformation efficiency. Two inactive TAP-Cas9-Cr1 and two inactive TAP-Cas9-Cr22 isolated from escaper clones were sequenced.

CSTB Algorithm

The CSTB web service enables the comparative analysis of CRISPR motifs across a wide range of bacterial genomes and plasmids. Currently considered motifs are NGG-anchored sequences of 18 to 23 base pairs long. The CSTB back-end database indexes all occurrences of these CRISPR motifs present in 2914 complete genomes labeled as representative or reference in the release 99 of RefSeq (Mar. 12, 2020). In addition, 7 bacterial genomes and 5 plasmids of interest were added. The mean number of distinct motifs among bacterial genomes is 55923 (5719 and 2729570 as respective minimum and maximum). Genomes are classified according to the NCBI taxonomy (Jul. 22, 2020). Each genome is inserted in the database of motifs by processing the corresponding complete fasta using the following procedure. Firstly, all words satisfying the CRISPR motif regular expression are detected and their chromosomal coordinates stored in a database of motifs. Secondly, all unique words are converted into an integer representation using a 2-bits per base encoding software we developed [https://github.com/glaunay/crispr-set]. These integers are then sorted in a unique flat file per genome. The indexing of CRISPR motifs as integers enables computationally efficient comparison of the sets of motifs across several organisms. Finally, the original fasta file is added to a blast database. All related software can be freely accessed at https://github.com/MMSB-MOBI/CSTB_database_manager. The CSTB input interface displays the 2914 genomes available for searching as two taxonomic trees. The left-hand tree allows for the selection of species whose genomes have to feature identical/similar CRISPR motifs. This set of genomes defines the targeted CRISPR motifs. Meanwhile, the right-hand tree allows for the selection of “excluded” organisms, which must have no motif in common with the targeted ones. The set of motifs that satisfies the user selections will effectively be equal to the union of the motifs found in the selected organisms subtracted from the intersection of the motifs found in the “excluded” organisms. Computation time ranges from seconds to a few minutes according to the size of the selections and an email is sent upon completion.

All the solutions CRISPR motifs are presented in an interactive table of sgRNA sequences and their occurrences in each selected organism. The table has sorting and filtering capabilities on motif counts and sequence composition. This allows for the easy selection of motifs of interest. Detailed information can be downloaded for the entire set of solutions or for the selected motifs only. This detailed information is provided in tabulated file with lines featuring the coordinates of each sgRNA motif in the targeted organisms. Alternatively, the user may explore the results using a genome-based approach. Hence, each targeted genome has its graphical view. The graphic is a circular histogram of the entire distribution of solution sgRNA motifs in a selected genome. The graphic is interactive to display the local breakdown of sgRNA distribution. The CSTB web site can be freely accessed at https://cstb.ibcp.fr.

Results:

Targeted-Antibacterial-Plasmids (TAPs) Modular Design

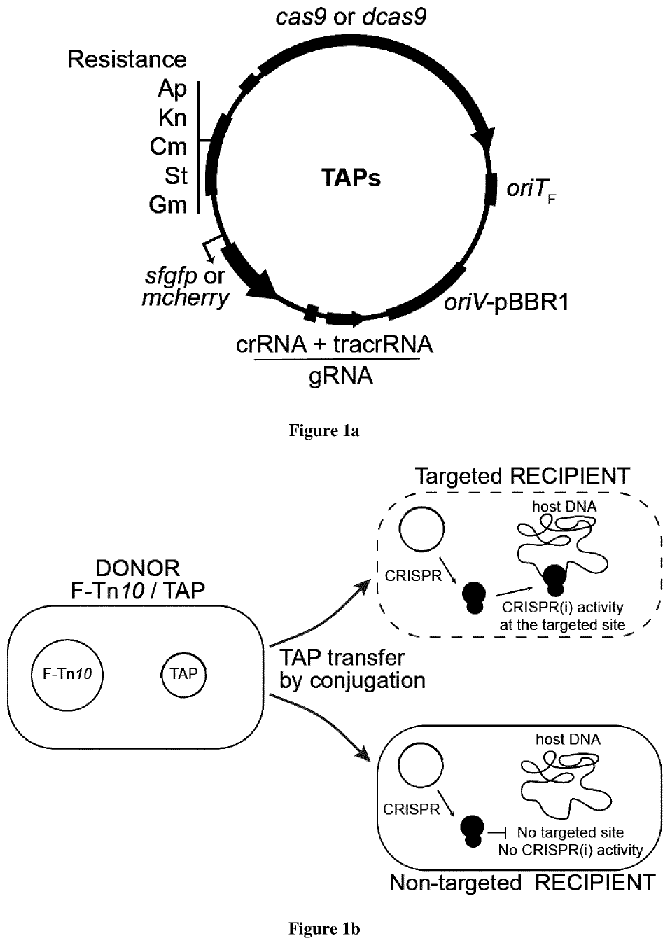

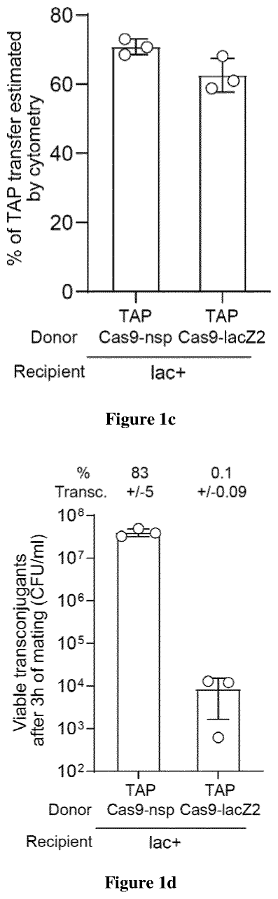

TAPs derivate from the synthetic pSEVA plasmid collection⁷, and carry the pBBR1 origin of replication, a choice of resistant gene cassettes, and the oriT_{F} origin of transfer of the F plasmid (FIG. 1a). TAPs are consequently mobilizable by the conjugation machinery produced in trans from the conjugative F-Tn10 helper plasmid contained in the donor cells (FIG. 1b)^{8,9}. We inserted the Streptococcus pyogenes wild-type cas9 (for CRISPR activity) or catalytically dead dcas9 gene (for CRISPRi activity) and the guide gRNA sequence composed of the constant tracrRNA scaffold and the target-specific crRNA spacer sequence (Fi 0.1a). Changing the crRNA spacer sequence in one-step-cloning allows reprogramming the targeting of the TAPs against any specific chromosome or plasmid DNA. Optionally, TAPs also carry either the superfolder greenfluorescent (sfgfp) or the mcherry gene highly expressed from the broad-host range synthetic BioFab promoter¹⁰ to serve as plasmid transfer fluorescent reporter in microscopy and flow cytometry assays (FIG. 1a).

Validation of TAPs CRISPR and CRISPRi Activities

We addressed the ability of TAPs to induce efficient and specific Cas9-mediated killing (CRISPR) or dCas9-mediated gene expression inhibition (CRISPRi). First, TAPs ability to induce Cas9-mediated killing was confirmed using the previously described lacZ2 spacer that targets the lacZ gene of E. coli¹¹. Transformation of the TAP-Cas9-lacZ2 plasmid into the lac+MG1655 wt strain was ˜1000-fold less efficient than in the isogenic lac− strain carrying a deletion of the targeted lacZ locus. By contract, the TAP-Cas9-nsp plasmid, which contains a non-specific (nsp) crRNA spacer that does not target E. coli genome, was transformed with equal efficiency in both lac+ and lac− strains. Second, TAPs ability to induce dCas9-mediated CRISPRi activity was validated by using the csgB spacer that targets the csgB promoter driving the production of cell-surface curlifimbriaei² in the MG1655 E. coli mutant strain OmpR234¹³. Congo Red (CR) staining on agar-plates and aggregation clumps formation in liquid medium were used as direct readouts for curli production^{13,14}. The TAP-dCas9-csgB efficiently inhibits curli production by the OmpR234 strain, as reflected by the formation of white colony in the presence of CR and the inability to form aggregation clumps. By contrast, the non-specific TAP-dCas9-nsp add no effect on curli formation or aggregation in the OmpR234 strain. Besides, we confirmed that the constitutive production of the Cas9 or dCas9 from the TAPs do not cause growth defects or elongated cell morphology, contrasting with the toxic effects reported in some systems¹⁵⁻¹⁸. These results demonstrate that TAPs ability to induce Cas9-mediated killing or dCas9-mediated gene expression inhibition is efficient and depends on the accurate targeting by the spacer sequence.

TAPs-Mediated Killing of Targeted Recipient Cells

Next, we addressed the ability of the TAPs to be transferred by conjugation and induce antibacterial activity in E. coli recipient cells. Conjugation was performed using the E. coli MG1655 donor strain that contains the F-Tn10 helper plasmid and either the TAP-Cas9-nsp or TAP-Cas9-lacZ2 mobilizable plasmids. Using flow cytometry analysis, we quantified the transfer efficiency of these TAPs (carrying the sfGFP green fluorescent reporter) into a lac+ recipient strain that produces the red fluorescent histone-like protein HU-mCherry, encoded on the chromosome. Quantification of the transconjugants exhibiting combined red and green fluorescence show that TAP-Cas9-nsp and TAP-Cas9-lacZ2 are both transferred to ˜65% of the recipient cell population after 3 h of mating (FIG. 1c). As expected, TAPs transfer requires the presence of the F-Tn10 plasmid in the donor strain. Most importantly, the parallel plating of the conjugation mixes revealed a ˜3.5-log decrease in the viability of TAP-Cas9-lacZ2 transconjugants compared to TAP-Cas9-nsp transconjugants (FIG. 1d). This killing activity is also reflected by the lack of increase in the total recipient cells count during the three hours of mating with the TAP-Cas9-lacZ2 donor strain, compared to a ˜20-fold increase with TAP-Cas9-nsp donors (FIG. 1e). Importantly, no killing effect is observed for either TAPs when using the isogenic lac− recipient strain lacking the targeted lacZ locus. These results show that TAP-Cas9-nsp and TAP-Cas9-lacZ2 are transferred with equal efficiency through the F-Tn10 conjugation machinery. Yet, the acquisition of TAP-Cas9-lacZ2, but not TAP-Cas9-nsp, is associated with a loss of viability of the transconjugant cells.

Using live-cell microscopy, we characterized the cellular response of the recipient cells to the acquisition of TAPs (FIG. 2). In these experiments, the TAPs carry the sfGFP reporter system that confers green fluorescence to the donor and transconjugant cells. The lac+ recipient cells produce the nucleoid-association protein HU-mCherry, which localization reveals the global organization of the chromosome. As expected, the acquisition of the TAP-Cas9-nsp reported by the production of sfGFP green fluorescence in red recipient cells has no impact on growth, cell morphology or nucleoid organization (not shown). By contrast, the acquisition of the TAP-Cas9-lacZ2 triggers the rapid disorganization of the nucleoid that grows into an unstructured DNA bulk, followed by cells filamentation and occasional cell lysis (FIG. 2a-b). Furthermore, we analyzed in recipient cells the localization pattern of a RecA-GFP fusion, which has been reported to polymerize into large intracellular structures in response to DNA-damage induction¹⁹. In this experiment, TAPs carry the mCherry reporter system that confers red fluorescence to donors and transconjugant cells. Image analysis reveals that the acquisition of the TAP-Cas9-lacZ2 (not shown), but not the TAP-Cas9-nsp (not shown), is followed by cells filamentation (FIG. 2c) as well as the RecA-GFP polymerization, which was quantified using fluorescence skewness analysis (FIG. 2d, see methods). Nucleoid disorganization, cell filamentation and RecA-GFP bundle formation confirm that TAP-Cas9-lacZ2 acquisition is followed by CRISPR-mediated induction of DSBs that result in the death of the transconjugants.

TAPs-Mediated Selective Killing within a Mixed E. coli Recipient Population

We verified the specificity of TAPs-mediated killing within a mixed recipient population composed of the targeted lac+ and the non-targeted lac− E. coli strains. We observe a ˜4 log-fold decrease in viable lac+ transconjugants compared to lac− transconjugants when using the TAP-Cas9-lacZ2, while no difference is observed with the TAP-Cas9-nsp (FIG. 3a). TAP-Cas9-lacZ2 specific killing activity is also reflected by the stagnation of the targeted lac+ recipient total population, while the non-targeted lac− population is able to grow during the six hours of mating (FIG. 3b). We performed live-cell microscopy imaging where the lac+ and lac− recipients are distinguished by the typical localization pattern of nucleoid associated HU-mCherry and the replisome associated DNA clamp DnaN-mCherry, respectively. Time-lapse analysis shows that both strains receive the plasmids reported by the increase of green fluorescence, yet only the targeted lac+ transconjugants exhibits cell filamentation, symptomatic of Cas9-mediated DNA-damage induction (FIG. 3c). These results recapitulate the effects obtained when using individual recipient strains, and demonstrate that the TAPs achieve selective killing of the targeted strain within a mixed population.

Analysis of TAP-Escape Mutants

Transfer of the TAP-Cas9-lacZ2 is associated with a ˜3.5-log viability loss of the lac+ transconjugant cells, yet we noticed a proportion of transconjugants that are able to survive despite the acquisition of the TAP (FIG. 1d). Genotyping and sequence analysis of 31 clones escaping the TAP-Cas9-lacZ2 activity revealed two types of escape mutants. One third (12 out of 31) have acquired a transposase or IS insertion in the plasmid-born cas9 gene, thus inactivating the CRISPR system. Two-third have acquired mutations that modify the targeted lacZ chromosome locus, either by small or large deletions (12 out of 31) as already described¹¹, or by single point mutation in the seed region of the PAM (7 out of 31), which was shown to be key for recognition by the Cas9-gRNA complex²⁰.

TAPs Directed Against Carbapenem-Resistant Population

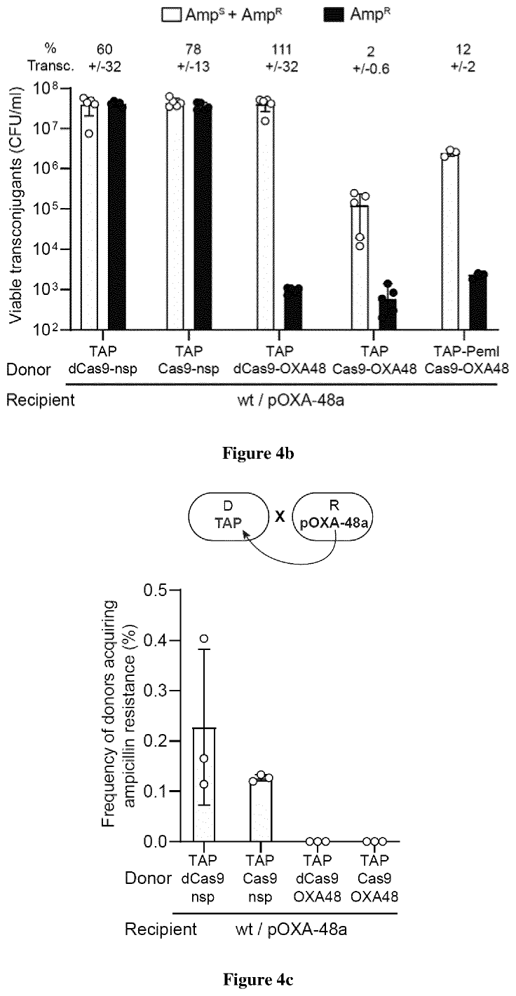

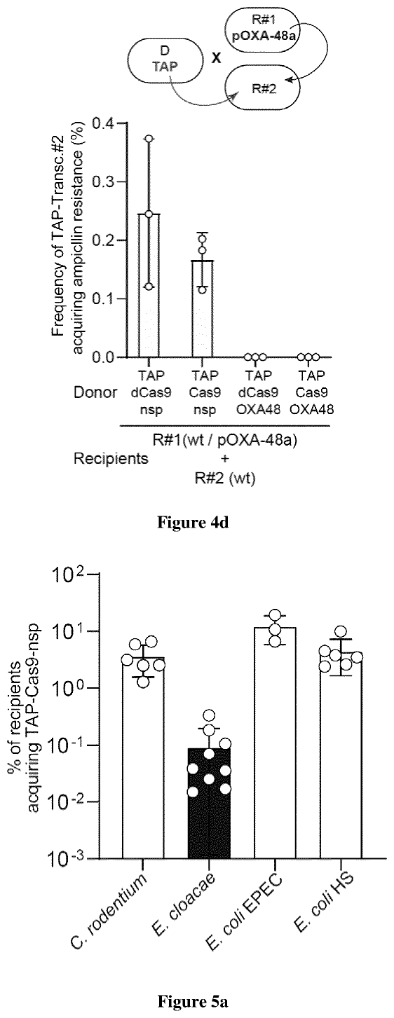

Conjugative plasmids are major contributors to the spread of multi-drug resistance in bacteria²¹, those conferring carbapenem resistance being of severe clinical concern²². The IncL/M pOXA-48a plasmid carries the bla_{OXA-48} gene that encodes the OXA-48 carbapenemase, which confer resistance to carbapenem and other beta lactams, such as imipenem and penicillin²³. We designed TAPs targeting the pOXA-48a and assessed their ability to sensitize the plasmid-carrying population to ampicillin. Using an OXA48 spacer that targets the 5′-end of the bla_{OXA-48} gene, we constructed the TAP-Cas9-OXA48 to induce Cas9-mediated DSBs on pOXA-48a, and the TAP-dCas9-OXA48 to inhibit bla_{OXA-48} gene transcription by CRISPRi (FIG. 4a). Transfer of TAP-Cas9-OXA48 and TAP-dCas9-OXA48 plasmids into pOXA-48a-carrying E. coli recipients lead to a ˜4.5-log decrease in ampicillin resistance level, while the TAP-Cas9-nsp or the TAP-dCas9-nsp have no effect (FIG. 4b). Unexpectedly, we observe that significantly less viable transconjugant are obtained without ampicillin selection when using the TAP-Cas9-OXA48 compared to TAP-dCas9-OXA48 (FIG. 4b). We ruled out the possibility of a decrease in TAP-Cas9-OXA48 transfer ability as all four tested plasmids are acquired with similar frequency by pOXA-48a plasmid-free E. coli recipients. However, analysis of the pOXA-48a plasmid sequence revealed the presence of the pemIK toxin-antitoxin (TA) system, which is involved in plasmid stability by inhibiting the growth of daughter cells that do not inherit the plasmid²⁴⁻²⁶. Indeed, the arrest of pemIK expression due to plasmid loss results in the rapid depletion of the labile PemI antitoxin, which can no longer repress the toxic activity of the more stable PemK toxin. This regulation was reported using CRISPR-associated phage therapy to cure antibiotic resistance carried by the pSHV-18 plasmid²⁶. We then hypothesized that the observed reduction of viable TAP-Cas9-OXA48 transconjugants could be due to PemK toxic activity in cells that have lost of the pOXA-48a targeted by the Cas9 cleavage. This possibility was confirmed by inserting a constitutively expressed antitoxin pemI gene into the TAP-Cas9-OXA48, which results in a ˜1.5 log increase in transconjugants viability, while retaining the inhibition of ampicillin resistance (FIG. 4b).

We further investigated the long-term evolution of resistance of the strain carrying the pOXA-48a conjugation with a TAP donor. We observed that while the TAP-dCas9-nsp had no effect, the transfer of the TAP-dCas9-OXA48 and the resulting re-sensibilization of the recipient population to ampicillin reached equilibrium after 24 h. From this point on, a stable 90% of the recipients have received the TAP-dCas9-OXA48 and became sensitive to ampicillin. We hypothesized that the remaining 10% of ampicillin-resistant recipient cells could results from the acquisition of the F-Tn10 plasmid only, thus resulting in the establishment of the F-encoded exclusion systems in the recipient cells and the permanent inability to acquire the TAP through a subsequent conjugation event. This hypothesis was confirmed by showing that all ampicillin-resistant recipients present in the population after 1 and 7 days of co-culture do contain the F-Tn10 but not the TAP-dCas9OXA47. One way to modulate the transfer efficiency of the mobilizable TAPs would be to prevent the transfer of the F plasmid by deletion of its origin of transfer. First, this would prevent the acquisition of the F plasmid only and the consequent establishment of the exclusion mechanism in the recipient cells. Second, the recipient cells that receive the TAP only would be unable to transmit it to other recipient bacteria due to the absence of the F-encoded conjugation machinery. In this situation TAPs are expected to disseminate more slowly, but potentially to all recipient cells in the population.

The pOXA-48a is an autonomous conjugative plasmid that disseminates among Enterobacteriaceae, raising the possibility that the recipient containing the pOXA-48a could transfer ampicillin resistance to the TAPs-donors during mating. We observed that ampicillin resistance is indeed transmitted to ˜0.2% and 0.12% of donors carrying the TAP-dCas9-nsp or the TAP-Cas9-nsp (FIG. 4c). However, donors carrying the TAP-dCas9-OXA48 or the TAP-Cas9-OXA48 do not acquire ampicillin resistance (FIG. 4c). Assuming that the efficiency of pOXA48 transfer is insensitive to the presence of the TAPs in the cells, this result suggests that TAPs directed against OXA48 impedes the development of resistance, even if the pOXA48 plasmid is acquired. We tested this possibility by performing the same conjugation experiments with an additional plasmid-free recipient wt strain (R #2) in the conjugation mix (see diagram in FIG. 4d). Among R #2 cells that have received the TAP-Cas9-nsp or the TAP-dCas9-nsp, ˜0.24% and 0.15% become ampicillin resistant, respectively. However, no ampicillin resistance is observed in R #2 cells that have received the TAP-Cas9-OXA48 or the TAP-dCas9-OXA48 (FIG. 4d). Altogether, these results demonstrate that directing TAPs against the bla_{OXA-48} gene is an efficient strategy to sensitize the pOXA-48a-carrying strain to ampicillin. In addition, TAPs also appear to impede drug-resistance dissemination by protecting the donor and other plasmid-free recipients from developing the resistance.

CSTB Software: Targeting Specific Strains within Multispecies Bacterial Population

Designing TAPs that perform antibacterial activity against specific bacterial species, without affecting other bacterial strains, requires the robust identification of spacer sequences that are present in the genome of the targeted organism(s), but not in the genomes of other non-targeted strains. Since no such tools existed to achieve this task, we developed a “Crispr Search Tool for Bacteria” CSTB algorithm, that enables the comparative analysis of ˜18-23 nt long spacer sequences across a wide range of bacterial genomes and plasmids. The CSTB back-end database indexes all occurrences of these motifs present in 2919 complete genomes classified according to the NCBI taxonomy. CSTB allow identifying appropriate spacer sequences to reprogram TAPs against unique or multiple sites in the targeted chromosome or plasmid DNA. We asked the CSTB algorithm to generate spacer sequences that target the attachment/effacement (A/E) pathogen Citrobacter rodentium strain ICC168 (Cr spacers), or the enteropathogenic E. coli EPEC strain E2348/69 (EPEC spacer), or the nosocomial pathogen Enterobacter cloacae (Ec1 spacer), or the three of them (EEC spacer), without targeting any other bacterial genome present in the database. TAPs directed against C. rodentium carry a Cr1 spacer that target a unique locus, or a Cr22 that targets 22 loci distributed throughout the genome. Transfer of TAP-Cas9-Cr1 from an E. coli donor reduces by 4-log the viability of C. rodentium transconjugant cells. Live-cell microscopy revealed that TAP-Cas9-Cr1 acquisition induces C. rodentium filamentation and lysis, while no growth defect was induced by the TAP-Cas9-nsp. This indicates that, as observed in E. coli, the induction of a single DSB by the Cas9 is lethal to C. rodentium. Consistently, targeting 22 chromosome loci by the TAP-Cas9-Cr22 results in comparable transconjugant killing efficiency. However, multiple targeting unbalances the contribution of the mechanisms by which transconjugants escape to the TAPs activity. Analysis of twenty clones escaping Cr1 single targeting revealed either deletions of the targeted chromosomal locus or inactivation of the CRISPR system on the TAP, in equal proportion. By contrast, the vast majority (19 out of 20) of clones escaping the Cr22 multiple targeting carry mutations that inactivate the TAP CRISPR system. This is consistent with the prediction that mutations of the 22 targeted chromosome sites within the same call is highly infrequent, if even possible.

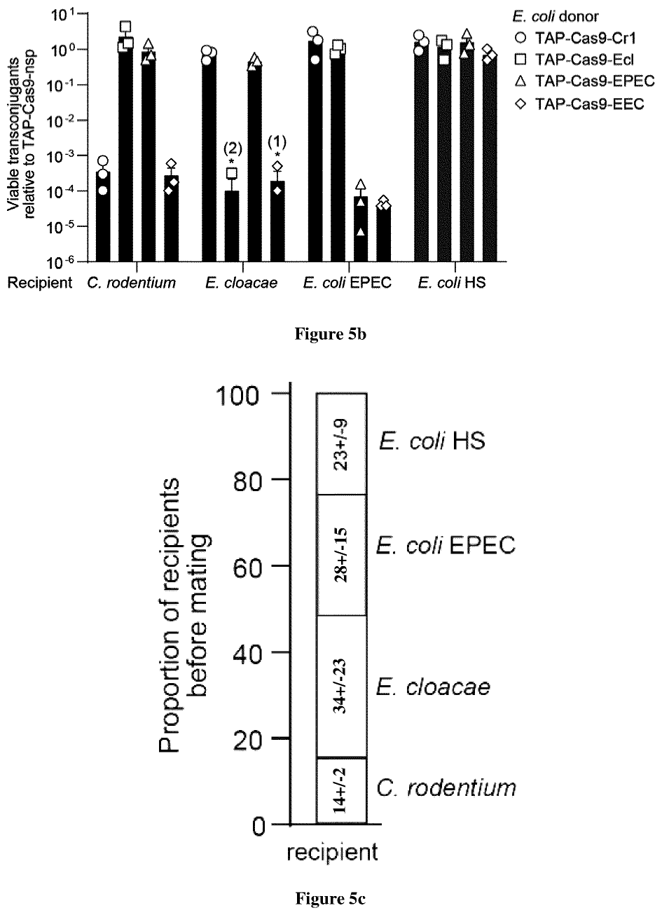

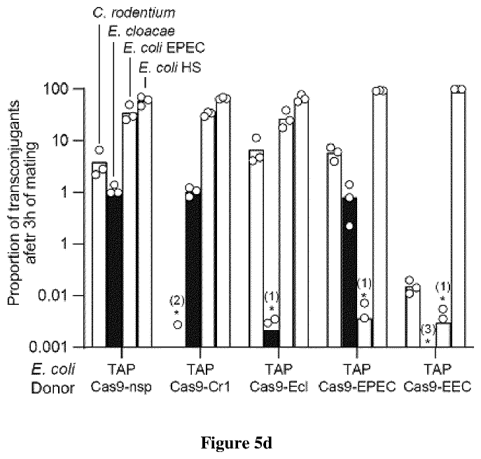

TAP transfer through F conjugation machinery is highly efficient towards MG1655 E. coli laboratory strain reaching up to 90% efficiency in 3 h of mating (FIG. 1). We quantified the efficiency of TAP-Cas9-nsp transfer in non-laboratory strain and observed a disparity between recipients with an overall ˜7- to 900-fold decrease in TAP acquisition frequency in comparison to MG1655 E. coli (FIG. 5a). To account for this variability, we normalized the frequency of viable transconjugants obtained for TAP-Cas9-Cr1, -Ec1, -EPEC and -EEC to the frequency of TAP-Cas9-nsp transconjugants in the corresponding bacterial strain (FIG. 5b). We quantified that the TAP-Cas9-Cr1 induces a transconjugant viability loss only in C. rodentium, TAP-Cas9-Ec1 in E. cloacae, TAP-Cas9-EPEC in E. coli EPEC, while the TAP-Cas9-EEC targets the three pathogenic strains. As a control, we show that the commensal E. coli HS recipient, which genome is not targeted by any spacer, is affected by none of these antibacterial TAPs (FIG. 5b). These results demonstrate that the spacer sequences generated by the CSTB algorithm allow the robust reprograming of the TAPs for efficient and strain-specific antibacterial activity on mono-species recipient populations. It also demonstrates that one given TAP can target several species at the time.

Next, we addressed TAPs ability to induce strain-specific antibacterial activity within a multi-species recipient population composed of an equal proportion the three pathogenic strains and the commensal E. coli HS (FIG. 5c). The proportion of transconjugants obtained after 3 h of mating with TAP-Cas9-nsp varies (FIG. 5d) and reflects the efficiency of TAP transfer among the different recipient strains (FIG. 5a). We observed that within the multispecies recipient mix, C. rodentium transconjugant viability is dramatically reduced by TAP-Cas9-Cr1, that of E. cloacae by TAP-Cas9-Ec1 and that of E. coli EPEC by TAP-Cas9-EPEC, while all three species are affected by the triple-targeting TAP-Cas9-EEC. The viability of transconjugants of the control commensal E. coli HS is not affected by any of the antibacterial TAPs (FIG. 5d). These results validate that TAPs achieve selective killing within a multispecies mixed recipient population without affecting the non-targeted species. Although the antibacterial TAPs impact selectively the viability of the transconjugant populations, their activity is not significantly reflected by the total recipient counts of each species, due to the limited efficiency of TAP transfer to the pathogenic recipient strains and the differential fitness of these strains in competition within the conjugation mix.

### Discussion

Tools for in situ microbiota manipulation are currently in their infancy. Here we demonstrate the ability of the TAP antibacterial strategy to exert an efficient and strain-specific antibacterial activity within multi-species populations in vitro. TAPs selective-killing activity induces a ˜4-log viability loss of the tested species. TAPs targeting the pOXA48a carbapenem resistance-plasmids results in a 4- to 5-logs increase of the strain susceptibility to the drug. Most CRISPR delivery methodology currently in development focus on the use of bacteriophages, which have intrinsically narrow host-range²⁷. Besides, several recent studies successfully use the broad host range RK2 conjugation systems to deliver CRISPR system that target E. coli^{26,28-30} or S. enterica³¹ in vitro. One key advantage of our strategy over these approaches is the versatility conferred by the CSTB algorithm that enables the robust identification of gRNA that should be used to specifically re-target the TAPs against one or several bacterial strains of interest, without targeting other species. Despite the availability of numerous programs dedicated to the identification of CRISPR motifs, the CSTB has no equivalent so far³². TAPs can rapidly and easily be reprogrammed by changing the spacer sequence in one-step-cloning (see methods). Another advantage of TAPs is the constitutive expression of the CRISPR system (and the fluorescent reporters) from promoter that are active in a wide range of Enterobacteriaceae. The absence of requirement for an external inductor renders the TAP approach more suitable for the modification of natural bacterial communities in vivo.

Our work also reveals that TAPs efficiency is primarily determined by two main limiting factors. The first limiting factor is the ˜10⁻⁴-10⁻⁵ frequency of escaper clones that acquire mutations inactivating the plasmid-born cas9 gene, or mutations that modify the targeted sequence. As shown in C. rodentium, the latter escape mechanism can be avoided by targeting multiple sites on the genome of the targeted bacteria. Another strategy would be to target essential genes, as their mutation is often lethal for the host bacteria³³. The second limiting parameter is the efficiency of TAPs transfer towards the targeted strain(s). So far, all of the antibacterial^{28,34} or anti-drug^{29,30,35} methodology using conjugation are based on the incP RK2 conjugative system, which offer broad-host range, but low efficiency of transfer (10⁻⁴-10⁻⁵). Hamilton et al. have shown an artificial way to increase the efficiency of transfer using glass beads in vitro³¹. Here, we use the F plasmid as a helper plasmid that mediates relatively efficient TAPs transfer (10⁻¹-10²) to closely related Enterobacteriaceae. Therefore, TAPs appear appropriate to target a range of clinically relevant pathogenic or resistant bacteria (E. coli, Citrobacter, Enterobacter, Klebsiella, Salmonella, Yersinia, Shigella, Serratia . . . ). Using TAPs to target phylogenetically distant species would require the development of broad-host range conjugation systems with increased transfer ability. Such superspreader plasmid mutants have been successfully isolated through Tn-seq approach^{36,37} and could represent an valuable option to widen the range of bacteria toward which TAPs could be directed.

Translating the present in vitro proof of concept to in situ settings would represent an important step towards the development of a non-antibiotic strategy for the in situ manipulation of microbiota composition, in a directed manner. TAPs could be used for the inhibition of harmful pathogenic and resistance strains from an infected host or environments, or as anti-virulence strategy through inhibition of virulence effector genes or genes involved in biofilm formation. The future applicability of the TAPs approach in clinical or environmental settings would require the consideration of the rapidly evolving regulations on GMO, CRISPR and biocontainment³⁸⁻⁴⁰.

### REFERENCES

Throughout this application, various references describe the state of the art to which this invention pertains. The disclosures of these references are hereby incorporated by reference into the present disclosure.
1. WHO. Global priority list of antibiotic—resistant bacteria to guide research, discovery, and development of new antibiotics. (2017).
2. Jinek, M. et al. A programmable dual-RNA-guided DNA endonuclease in adaptive bacterial immunity. Science (New York, N.Y.) 337, 816-821 (2012).
3. Gasiunas, G., Barrangou, R., Horvath, P. & Siksnys, V. Cas9-crRNA ribonucleoprotein complex mediates specific DNA cleavage for adaptive immunity in bacteria. Proc Natl Acad Sci USA 109, E2579-2586 (2012).
4. Qi, L. S. et al. Repurposing CRISPR as an RNA-Guided Platform for Sequence-Specific Control of Gene Expression. Cell 152, 1173-1183 (2013).
5. Bikard, D. et al. Programmable repression and activation of bacterial gene expression using an engineered CRISPR-Cas system. Nucleic Acids Research 41, 7429-7437 (2013).
6. Anders, C., Niewoehner, O., Duerst, A. & Jinek, M. Structural basis of PAM-dependent target DNA recognition by the Cas9 endonuclease. Nature 513, 569-573 (2014).
7. Martinez-Garcia, E., Aparicio, T., Goni-Moreno, A., Fraile, S. & de Lorenzo, V. SEVA 2.0: an update of the Standard European Vector Architecture for de-/re-construction of bacterial functionalities. Nucleic Acids Res. 43, D1183-1189 (2015).
8. Nolivos, S. et al. Role of AcrAB-TolC multidrug efflux pump in drug-resistance acquisition by plasmid transfer. Science 364, 778-782 (2019).
9. Crisona, N. J. & Clark, A. J. Increase in conjugational transmission frequency of nonconjugative plasmids. Science 196, 186-187 (1977).
10. Mavridou, D. A. I., Gonzalez, D., Clements, A. & Foster, K. R. The pUltra plasmid series: A robust and flexible tool for fluorescent labeling of Enterobacteria. Plasmid 87-88, 65-71(2016).
11. Cui, L. & Bikard, D. Consequences of Cas9 cleavage in the chromosome of Escherichia coli. Nucleic Acids Res. 44, 4243-4251 (2016).
12. Bhoite, S., van Gerven, N., Chapman, M. R. & Remaut, H. Curli Biogenesis: Bacterial Amyloid Assembly by the Type VIII Secretion Pathway. EcoSal Plus 8, (2019).
13. Vidal, O. et al. Isolation of an Escherichia coli K-12 mutant strain able to form biofilms on inert surfaces: involvement of a new ompR allele that increases curli expression. J. Bacteriol. 180, 2442-2449 (1998).
14. Serra, D. O. & Hengge, R. Experimental Detection and Visualization of the Extracellular Matrix in Macrocolony Biofilms. in c-di-GMP Signaling (ed. Sauer, K.) vol. 1657 133-145 (Springer New York, 2017).
15. Rock, J. M. et al. Programmable transcriptional repression in mycobacteria using an orthogonal CRISPR interference platform. Nature Microbiology 2, (2017).
16. Cho, S. et al. High-Level dCas9 Expression Induces Abnormal Cell Morphology in Escherichia coli. ACS Synthetic Biology 7, 1085-1094 (2018).
17. Zhang, S. & Voigt, C. A. Engineered dCas9 with reduced toxicity in bacteria: implications for genetic circuit design. Nucleic Acids Research (2018) doi:10.1093/nar/gky884.
18. Misra, C. S. et al. Determination of Cas9/dCas9 associated toxicity in microbes. http://biorxiv.org/lookup/doi/10.1101/848135 (2019) doi:10.1101/848135.
19. Lesterlin, C., Ball, G., Schermelleh, L. & Sherratt, D. J. RecA bundles mediate homology pairing between distant sisters during DNA break repair. Nature 506, 249-253 (2014).
20. Semenova, E. et al. Interference by clustered regularly interspaced short palindromic repeat (CRISPR) RNA is governed by a seed sequence. Proceedings of the National Academy of Sciences 108, 10098-10103 (2011).
21. Barlow, M. What antimicrobial resistance has taught us about horizontal gene transfer.

Methods Mol. Biol. 532, 397-411 (2009).
22. Codjoe, F. & Donkor, E. Carbapenem Resistance: A Review. Medical Sciences 6, 1 (2017).
23. Poirel, L., Bonnin, R. A. & Nordmann, P. Genetic Features of the Widespread Plasmid Coding for the Carbapenemase OXA-48. Antimicrobial Agents and Chemotherapy 56, 559-562 (2012).
24. Hayes, F. Toxins-antitoxins: plasmid maintenance, programmed cell death, and cell cycle arrest. Science 301, 1496-1499 (2003).
25. Mnif, B. et al. Molecular characterization of addiction systems of plasmids encoding extended-spectrum beta-lactamases in Escherichia coli. J. Antimicrob. Chemother. 65, 1599-1603 (2010).
26. Citorik, R. J., Mimee, M. & Lu, T. K. Sequence-specific antimicrobials using efficiently delivered RNA-guided nucleases. Nature Biotechnology 32, 1141-1145 (2014).
27. Bikard, D. & Barrangou, R. Using CRISPR-Cas systems as antimicrobials. Curr. Opin.

Microbiol. 37, 155-160 (2017).
28. Ji, W. et al. Specific gene repression by CRISPRi system transferred through bacterial conjugation. ACS Synth Biol 3, 929-931 (2014).
29. Dong, H., Xiang, H., Mu, D., Wang, D. & Wang, T. Exploiting a conjugative CRISPR/Cas9 system to eliminate plasmid harbouring the mcr-1 gene from Escherichia coli.

Int. J. Antimicrob. Agents 53, 1-8 (2019).
30. Ruotsalainen, P., Penttinen, R., Mattila, S. & Jalasvuori, M. Midbiotics: conjugative plasmids for genetic engineering of natural gut flora. Gut Microbes 1-11 (2019) doi:10.1080/19490976.2019.1591136.
31. Hamilton, T. A. et al. Efficient inter-species conjugative transfer of a CRISPR nuclease for targeted bacterial killing. Nat Commun 10, 4544 (2019).
32. Alkhnbashi, O. S., Meier, T., Mitrofanov, A., Backofen, R. & Voß, B. CRISPR-Cas bioinformatics. Methods 172, 3-11 (2020).
33. Gomaa, A. A. et al. Programmable Removal of Bacterial Strains by Use of Genome-Targeting CRISPR-Cas Systems. mBio 5, (2014).
34. Citorik, R. J., Mimee, M. & Lu, T. K. Sequence-specific antimicrobials using efficiently delivered RNA-guided nucleases. Nat. Biotechnol. 32, 1141-1145 (2014).
35. Wang, P. et al. Eliminating mcr-1-harbouring plasmids in clinical isolates using the CRISPR/Cas9 system. Journal of Antimicrobial Chemotherapy 74, 2559-2565 (2019).
36. Yamaichi, Y. et al. High-resolution genetic analysis of the requirements for horizontal transmission of the ESBL plasmid from Escherichia coli 0104:H4. Nucleic Acids Res. 43, 348-360 (2015).
37. Poidevin, M. et al. Mutation in ESBL Plasmid from Escherichia coli 0104:H4 Leads Autoagglutination and Enhanced Plasmid Dissemination. Front Microbiol 9, 130 (2018).
38. Fellmann, C., Gowen, B. G., Lin, P.-C., Doudna, J. A. & Corn, J. E. Cornerstones of CRISPR-Cas in drug discovery and therapy. Nat Rev Drug Discov 16, 89-100 (2017).
39. Davison, J. & Ammann, K. New GMO regulations for old: Determining a new future for EU crop biotechnology. GM Crops Food 8, 13-34 (2017).
40. Brokowski, C. & Adli, M. CRISPR Ethics: Moral Considerations for Applications of a Powerful Tool. J Mol Biol 431, 88-101 (2019).

1. A Targeted-Antibacterial-plasmid (TAP) comprising i) an origin of replication, ii) an origin of transfer, iii) a genetically-engineered nucleic acid sequences encoding a nuclease and iv) one or more genetically-engineered nucleic acid sequence(s) encoding a guide RNA molecule.

2. The TAP of claim 1 wherein the origin of replication is an origin of replication of pBBR1 plasmid.

3. The TAP of claim 1 wherein the origin of transfer is an oriT of RP4 plasmid.

4. The TAP of claim 1 wherein the origin of transfer is an oriTF of F plasmid.

5. The plasmid TAP of claim 1 wherein the nuclease is a CRISPR-associated endonuclease.

6. The TAP of claim 5 wherein the CRISPR-associated endonuclease is a Cas9 nuclease comprising the amino acid sequence as set forth in SEQ ID NO: 4.

7. The TAP of claim 6, wherein a nucleic acid sequence encoding the Cas9 nuclease comprises the nucleic acid sequence of SEQ ID NO:7.

8. The TAP of claim 6, wherein the Cas9 nuclease is a defective Cas9 nuclease.

9. The TAP of claim 8 wherein the defective Cas9 nuclease is a Cas9 nickase that comprises the amino acid sequence as set forth in SEQ ID NO:5 or SEQ ID NO:6.

10. The TAP of claim 9 wherein a nucleic acid sequence encoding the Cas9 nickase comprises the nucleic acid sequence of SEQ ID NO:8.

11. The TAP of claim 1 wherein a nucleic acid sequence that encodes the nuclease is operatively linked to a weak constitutive promoter.

12. The TAP of claim 1 that comprises at 2, 3, 3, 4, 5, 6, 7, 8, 9, or 10 nucleic acid sequences encoding guide RNA molecule.

13. The TAP of claim 1 wherein the guide RNA molecule comprises a spacer sequence and a trans-activating RNA sequence.

14. The TAP of claim 13 wherein the spacer sequence targets an antibiotic resistance gene or is designed to generate a double-strand break (DSB) in a target sequence.

15. The TAP of claim 13 wherein the spacer sequence is encoded by a nucleic acid sequence selected from Table A.

16. The TAP of claim 1 wherein the one or more genetically-engineered nucleic acid sequence(s) that encode a guide RNA molecule are operatively linked to a strong constitutive promoter.

17. The TAP of claim 1 that optionally comprises one or more selection marker(s).

18. The TAP of claim 1 that consists of the nucleic acid sequence as set forth in SEQ ID NO:27 or 28.

19. A donor bacterial cell comprising a copy number of the TAP of claim 1.

20. The donor bacterial cell of claim 19 that also comprises a copy number of conjugative plasmids.

21. The donor bacterial cell of claim 20 that comprises a copy number of F factors and a copy number of TAPs that comprise an origin transfer of F plasmid.

22. The donor bacterial cell of claim 20 that comprises a copy number of RP4 plasmids and a copy number of TAPs that comprise an origin transfer of RP4 plasmid.

23. The donor bacterial cell of claim 19 that is non-pathogenic.

24. A method for killing a plurality of recipient bacterial cells, comprising
exposing said plurality of recipient bacterial cells to a plurality of donor bacterial cells that comprise a copy number of the Targeted-Antibacterial-Plasmid (TAP) of claim 1, wherein the TAP is configured to express the nuclease and one or more guide RNA molecules in said plurality of recipient bacterial cells, and wherein transfer and expression of said nuclease and said one or more guide RNA molecules in said plurality of recipient bacterial cells is lethal for said plurality of recipient bacterial cells.

25. The method of claim 24 wherein the recipient bacterial cells are pathogenic bacteria.

26. (canceled)

27. A method of treating an infection in a subject caused by a bacterial cell comprising an antibiotic resistance gene, comprising
administering to the subject a therapeutically effective amount of the antibiotic in combination with a therapeutically effective amount of bacterial donor cells comprising a copy number of Targeted-Antibacterial-Plasmids (TAPs) that encode one or more guide RNA molecules that target and inactivate the antibiotic resistance gene, thereby sensitizing the bacterial cell to said antibiotic.

28. A composition comprising an amount of the donor bacterial cells of claim 19.
